(19) United States
(12) Patent Application Publication
(10) Pub. No.: US 2025/0009877 A1
(43) Pub. Date: Jan. 9, 2025

(54) TREATMENT FOR CANCER

(71) Applicant: Seagen Inc., Bothell, WA (US)

(72) Inventors: Michael Schmitt, Bothell, WA (US); Shyra J. Gardai, Bothell, WA (US)

(21) Appl. No.: 18/292,662

(22) PCT Filed: Jul. 28, 2022
(86) PCT No.: PCT/US2022/074259

§ 371 Date: Jan. 26, 2024

Related U.S. Application Data
(60) Provisional application No. 63/227,578, filed on Jul. 30, 2021.
(60) Provisional application No. 63/248,190, filed on Sep. 24, 2021.

(51) Int. Cl. A61K 39/395; A61K 31/282; A61K 31/337; A61K 31/519; A61K 39/00; A61P 35/00; C07K 16/28

(52) U.S. Cl. CPC A61K 39/39558; A61K 31/282; A61K 31/337; A61K 31/519; A61P 35/00; C07K 16/2818; C07K 16/2878; A61K 2039/507; C07K 2317/24

(57) ABSTRACT

This disclosure relates to methods of treating cancer using a combination of an anti-CD40 antibody such as SEA-CD40, and an anti-PD-1 antibody such as pembrolizumab. The treatment can further include a chemotherapy.

### CLAIM OF PRIORITY

This application claims the benefit of U.S. Provisional Application No. 63/227,578, filed on Jul. 30, 2021 and U.S. Provisional Application No. 63/248,190, filed on Sep. 24, 2021. The disclosures of the prior applications are hereby incorporated by reference in their entirety.

### TECHNICAL FIELD

This disclosure relates to treatment of cancer using a combination of therapeutic agents.

### BACKGROUND

Treatment for cancer may involve administering more than one therapeutic agent. Various therapeutic agents have been tested either as single agent or as used in a combination therapy. Single and combination therapies have been explored for treating melanoma and non-small cell lung cancer (NSCLC) but have not been satisfactory.

Anti-CD40 antibodies have been tested as potential therapeutics for treating cancer. CD40, a member of the tumor necrosis factor (TNF) receptor superfamily, is expressed on a variety of cell types including normal and neoplastic B cells, interdigitating cells, basal epithelial cells and carcinomas. The interaction of CD40 with its ligand/antigen, CD40L (also referred to as CD154, gp39, and TRAP), induces immune responses. A few anti-CD40 antibodies have been tested in clinical trials, but none has been approved by the FDA to date.

KEYTRUDA® (pembrolizumab), developed by Merck and Co., Inc., (Kenilworth NJ, USA) is an FDA-approved antibody therapeutic. To date, KEYTRUDA® has been approved for the treatment of various tumor and cancer types including certain melanoma, non-small cell lung cancer (NSCLC), small cell lung cancer (SCLC), head and neck squamous cell cancer (HNSCC), and classical Hodgkin lymphoma (cHL), among others. The prescription label of KEYTRUDA® is accessible at, e.g., FDA's Approved Drug database.

Pembrolizumab, the active ingredient of KEYTRUDA®, is an anti-PD-1 antibody that binds to its ligand/antigen the Program Death receptor 1 (PD-1) and helps in the clearance of tumor cells by the immune system. PD-1 is an immunoglobulin superfamily member and negatively regulates antigen receptor signaling upon engagement of its ligands PD-L1 and/or PD-L2. Some cancers, however, are not responsive to anti-PD-1 or anti-PD-L1 treatments (Danaher P et al. J Immunother Cancer. 2018 Jun. 22; 6(1):63; Algazi et al. Cancer. 2016 Nov. 15; 122(21): 3344-3353).

### BRIEF SUMMARY

Embodiment 1. A method of treating a melanoma comprising administering to a patient having melanoma SEA-CD40 and pembrolizumab wherein SEA-CD40 and pembrolizumab are both administered in 42-day cycles.

Embodiment 2. The method of embodiment 1 wherein SEA-CD40 is administered twice in each 42-day cycle and pembrolizumab is administered once in each 42-day cycle.

Embodiment 3. The method of embodiment 2 wherein the first administration of SEA-CD40, the second administration of SEA-CD40 and the administration of pembrolizumab are on the following days of each 42-day cycle, respectively: day 1, day 21, and day 5; day 1, day 22, and day 5; day 1, day 23, and day 5; day 2, day 21, and day 5; day 2, day 22, and day 5; day 2, day 23, and day 5; day 3, day 21, and day 5; day 3, day 22, and day 5; day 3, day 23, and day 5; day 1, day 21, and day 6; day 1, day 22, and day 6; day 1, day 23, and day 6; day 2, day 21, and day 6; day 2, day 22, and day 6; day 2, day 23, and day 6; day 3, day 21, and day 6; day 3, day 22, and day 6; day 3, day 23, and day 6; day 1, day 21, and day 7; day 1, day 22, and day 7; day 1, day 23, and day 7; day 2, day 21, and day 7; day 2, day 22, and day 7; day 2, day 23, and day 7; day 3, day 21, and day 7; day 3, day 22, and day 7; day 3, day 23, and day 7; day 1, day 21, and day 8; day 1, day 22, and day 8; day 1, day 23, and day 8; day 2, day 21, and day 8; day 2, day 22, and day 8; day 2, day 23, and day 8; day 3, day 21, and day 8; day 3, day 22, and day 8; day 3, day 23, and day 8; day 1, day 21, and day 9; day 1, day 22, and day 9; day 1, day 23, and day 9; day 2, day 21, and day 9; day 2, day 22, and day 9; day 2, day 23, and day 9; day 3, day 21, and day 9; day 3, day 22, and day 9; day 3, day 23, and day 9; day 1, day 21, and day 10; day 1, day 22, and day 10; day 1, day 23, and day 10; day 2, day 21, and day 10; day 2, day 22, and day 10; day 2, day 23, and day 10; day 3, day 21, and day 10; day 3, day 22, and day 10; or day 3, day 23, and day 10.

Embodiment 4a. The method of any one of embodiments 1-3, wherein SEA-CD40 is administered on day 1 and day 21 of each 42-day cycle, and pembrolizumab is administered on day 8 of each 42-day cycle.

Embodiment 4b. The method of any one of embodiments 1-3, wherein SEA-CD40 is administered on day 1 and day 22 of each 42-day cycle, and pembrolizumab is administered on day 8 of each 42-day cycle.

Embodiment 5. The method of embodiment 1 or 2 wherein pembrolizumab is not administered in the first 42-day cycle and is administered each cycle starting from the second cycle.

Embodiment 6. The method of embodiment 5 wherein SEA-CD40 is administered twice in each 42-day cycle and pembrolizumab is not administered in the first cycle and is administered once in each 42-day cycle starting from the second cycle.

Embodiment 7. The method of embodiment 5 or 6 wherein the first administration of SEA-CD40, the second administration of SEA-CD40 in each 42-day cycle, and the administration of pembrolizumab in each 42-day cycle starting from the second cycle are on the following days of the 42-day cycles, respectively: day 1, day 21, and day 5; day 1, day 22, and day 5; day 1, day 23, and day 5; day 2, day 21, and day 5; day 2, day 22, and day 5; day 2, day 23, and day 5; day 3, day 21, and day 5; day 3, day 22, and day 5; day 3, day 23, and day 5; day 1, day 21, and day 6; day 1, day 22, and day 6; day 1, day 23, and day 6; day 2, day 21, and day 6; day 2, day 22, and day 6; day 2, day 23, and day 6; day 3, day 21, and day 6; day 3, day 22, and day 6; day 3, day 23, and day 6; day 1, day 21, and day 7; day 1, day 22, and day 7; day 1, day 23, and day 7; day 2, day 21, and day 7; day 2, day 22, and day 7; day 2, day 23, and day 7; day 3, day 21, and day 7; day 3, day 22, and day 7; day 3, day 23, and day 7; day 1, day 21, and day 8; day 1, day 22, and day 8; day 1, day 23, and day 8; day 2, day 21, and day 8; day 2, day 22, and day 8; day 2, day 23, and day 8; day 3, day 21, and day 8; day 3, day 22, and day 8; day 3, day 23, and day 8; day 1, day 21, and day 9; day 1, day 22, and day 9; day 1, day 23, and day 9; day 2, day 21, and day 9; day 2, day 22, and day 9; day 2, day 23, and day 9; day 3, day 21, and day 9; day 3, day 22, and day 9; day 3, day 23, and day 9; day 1, day 21, and day 10; day 1, day 22, and day 10; day 1, day 23, and day 10; day 2, day 21, and day 10; day 2, day 22, and day 10; day 2, day 23, and day 10; day 3, day 21, and day 10; day 3, day 22, and day 10; or day 3, day 23, and day 10.

Embodiment 8. The method of any one of embodiments 1-7 wherein pembrolizumab is administered intravenously.

Embodiment 9. The method of any one of embodiments 1-8 wherein pembrolizumab is administered at a dose of 400 mg.

Embodiment 10. The method of any one of embodiments 1-9 wherein the melanoma is relapsed and/or refractory melanoma.

Embodiment 11. The method of any one of embodiments 1-9 wherein the melanoma is uveal melanoma.

Embodiment 12. The method of any one of embodiments 1-9 wherein the melanoma is PD-(L)1-naïve melanoma.

Embodiment 13. The method of any one of embodiments 1-12 wherein the level of PD-L1 in a sample of the melanoma is less than 50% as measured by Total Proportion Score (TPS).

Embodiment 14. The method of any one of embodiments embodiment 1-13 wherein the level of PD-L1 in a sample of the melanoma is less than 1% as measured by TPS.

Embodiment 15. The method of any one of embodiments embodiment 1-13 wherein the level of PD-L1 in a sample of the melanoma is 1-49% as measured by TPS.

Embodiment 16. The method of any one of the embodiments 1-15 further comprising administering one of more chemotherapy agents to the patient.

Embodiment 17. The method of embodiment 16 wherein the one or more chemotherapy agents comprises gemcitabine, dacarbazine, temozolomide, paclitaxel, albumin-bound paclitaxel, carboplatin, and/or pemetrexed.

Embodiment 18. The method of embodiment 17 wherein the albumin-bound paclitaxel is nab-paclitaxel.

Embodiment 19. A method of treating a melanoma comprising administering to a patient having melanoma SEA-CD40 and pembrolizumab wherein SEA-CD40 and pembrolizumab are both administered in 21-day cycles.

Embodiment 20. The method of embodiment 19 wherein both SEA-CD40 and pembrolizumab are administered once in each 21-day cycle.

Embodiment 21. The method of embodiment 19 or 20 wherein SEA-CD40 and pembrolizumab are administered on the following days of each 21-day cycle, respective: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 2 and day 11, day 3 and day 4, day 3 and day 5, day 3 and day 6, day 3 and day 7, day 3 and day 8, day 3 and day 9, day 3 and day 10, day 3 and day 11, day 3 and day 12, day 4 and day 5, day 4 and day 6, day 4 and day 7, day 4 and day 8, day 4 and day 9, day 4 and day 10, day 4 and day 11, day 4 and day 12, day 4 and day 13, day 5 and day 6, day 5 and day 7, day 5 and day 8, day 5 and day 9, day 5 and day 10, day 5 and day 11, day 5 and day 12, day 5 and day 13, or day 5 and day 14.

Embodiment 22. The method of any one of embodiments 19-21, wherein SEA-CD40 is administered on day 1 of each 21-day cycle, and pembrolizumab is administered on day 8 of each 21-day cycle.

Embodiment 23. The method of embodiment 19 or 20, wherein pembrolizumab is not administered in the first 21-day cycle and is administered each cycle starting from the second cycle.

Embodiment 24. The method of embodiment 23 wherein SEA-CD40 is administered once in each 42-day cycle and pembrolizumab is not administered in the first cycle and is administered once in each 21-day cycle starting from the second cycle.

Embodiment 25. The method of embodiment 23 or 24 wherein the administration of SEA-CD40 in each 21-day cycle, and the administration of pembrolizumab are on the following days of the 21-day cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 2 and day 11, day 3 and day 4, day 3 and day 5, day 3 and day 6, day 3 and day 7, day 3 and day 8, day 3 and day 9, day 3 and day 10, day 3 and day 11, day 3 and day 12, day 4 and day 5, day 4 and day 6, day 4 and day 7, day 4 and day 8, day 4 and day 9, day 4 and day 10, day 4 and day 11, day 4 and day 12, day 4 and day 13, day 5 and day 6, day 5 and day 7, day 5 and day 8, day 5 and day 9, day 5 and day 10, day 5 and day 11, day 5 and day 12, day 5 and day 13, or day 5 and day 14.

Embodiment 26. The method of any one of embodiments 19-25 wherein pembrolizumab is administered intravenously.

Embodiment 27. The method of any one of embodiments 19-26 wherein pembrolizumab is administered at a dose of 400 mg.

Embodiment 28. The method of any one of embodiments 19-27 wherein the melanoma is relapsed and/or refractory melanoma.

Embodiment 29. The method of any one of embodiments 19-27 wherein the melanoma is uveal melanoma.

Embodiment 30. The method of any one of embodiments 19-27 wherein the melanoma is PD-(L)1-naïve melanoma.

Embodiment 31. The method of any one of embodiments 19-30 wherein the level of PD-L1 in a sample of the melanoma is less than 50% as measured by Total Proportion Score (TPS).

Embodiment 32. The method of any one of embodiments embodiment 19-31 wherein the level of PD-L1 in a sample of the melanoma is less than 1% as measured by TPS.

Embodiment 33. The method of any one of embodiments embodiment 19-31 wherein the level of PD-L1 in a sample of the melanoma is 1-49% as measured by TPS.

Embodiment 34. The method of any one of the embodiments 19-33 further comprising administering one of more chemotherapy agents to the patient.

Embodiment 35. The method of embodiment 34 wherein the one or more chemotherapy agents comprises gemcitabine, dacarbazine, temozolomide, paclitaxel, albumin-bound paclitaxel, carboplatin, and/or pemetrexed.

Embodiment 36. The method of embodiment 35 wherein the albumin-bound paclitaxel is nab-paclitaxel.

Embodiment 37. A method of treating non-squamous NSCLC comprising administering to a patient having non-squamous NSCLC SEA-CD40, pembrolizumab, carboplatin, and pemetrexed.

Embodiment 38. The method of embodiment 37 wherein SEA-CD40, pembrolizumab, carboplatin and pemetrexed are all administered in 21-day cycles.

Embodiment 39. The method of embodiment 38 wherein carboplatin is administered on day 1 of the first four cycles (cycles 1-4) and not administered in the following cycles.

Embodiment 40. The method of embodiment 38 or 39 wherein pemetrexed is administered on day 1 of each 21-day cycle.

Embodiment 41. The method of any one of embodiments 38-40 wherein SEA-CD40 and pembrolizumab are administered on the following days of each 21-day cycle, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 42. The method of any one of embodiments 38-41, wherein SEA-CD40 and pembrolizumab are administered on the following days of each 21-day cycle, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 43. The method of any one of embodiments 38-41, wherein SEA-CD40 is administered on day 3 of each 21-day cycle, and pembrolizumab is administered on day 1 of each 21-day cycle.

Embodiment 44. The method of any one of embodiments 38-40, wherein pembrolizumab is not administered in the first 21-day cycle and is administered each cycle starting from the second 21-day cycle.

Embodiment 45. The method of embodiment 44, wherein the administration of SEA-CD40 in each 21-day cycle, and the administration of pembrolizumab in each 21-day cycle except the first cycle, are on the following days of the 21-day cycles, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 46. The method of embodiment 44 or 45, wherein the administration of SEA-CD40 in each 21-day cycle, and the administration of pembrolizumab in each 21-day cycle except the first cycle, are on the following days of the 21-day cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 47. The method of any one of embodiments 38-46 wherein pembrolizumab is administered intravenously.

Embodiment 48. The method of any one of embodiments 38-47 wherein pembrolizumab is administered at a dose of 200 mg.

Embodiment 49. The method of any one of embodiments 38-48, wherein pemetrexed is administered at a dose of 500 mg per square meter.

Embodiment 50. The method of any one of embodiments 38-49, wherein carboplatin is administered at a dose of area under the concentration-time curve (AUC) 2-8 mg per milliliter per minute.

Embodiment 51. The method of any one of embodiments 38-50, wherein carboplatin is administered at a dose of area under the concentration-time curve (AUC) 2 mg, 3 mg, 4 mg, 5 mg, 6 mg, 7 mg, or 8 mg per milliliter per minute.

Embodiment 52. The method of any one of embodiments 38-51, wherein carboplatin is administered at 5 mg per milliliter per minute.

Embodiment 53. The method of any one of embodiments 38-52 wherein the level of PD-L1 in a sample of the non-squamous NSCLC is less than 50% as measured by Total Proportion Score (TPS).

Embodiment 54. The method of any one of embodiments embodiment 38-53 wherein the level of PD-L1 in a sample of the non-squamous NSCLC is less than 1% as measured by TPS.

Embodiment 55. The method of any one of embodiments embodiment 38-53 wherein the level of PD-L1 in a sample of the non-squamous NSCLC is 1-49% as measured by TPS.

Embodiment 56. The method of any one of the embodiments 38-55 further comprising administering gemcitabine, dacarbazine, temozolomide, paclitaxel, and/or albumin-bound paclitaxel.

Embodiment 57. The method of embodiment 56 wherein the albumin-bound paclitaxel is nab-paclitaxel.

Embodiment 58. A method of treating non-squamous NSCLC comprising administering to a patient having non-squamous NSCLC SEA-CD40, pembrolizumab, carboplatin, and pemetrexed, wherein SEA-CD40, carboplatin and pemetrexed are administered in 21-day cycles, and pembrolizumab is administered in 42-day cycles.

Embodiment 59. The method of embodiment 58 wherein carboplatin is administered on day 1 of the first four cycles (cycles 1-4) and not administered in the following cycles. Embodiment 60. The method of embodiment 58 or 59 wherein pemetrexed is administered on day 1 of each 21-day cycle.

Embodiment 61. The method of any one of embodiments 58-60 wherein SEA-CD40 and pembrolizumab are administered on the following days in each of their respective cycles, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 62. The method of any one of embodiments 58-61, wherein SEA-CD40 and pembrolizumab are administered on the following days in each of their respective cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 63. The method of any one of embodiments 58-61, wherein SEA-CD40 is administered on day 3 of each 21-day cycle, and pembrolizumab is administered on day 1 of each 42-day cycle.

Embodiment 64. The method of any one of embodiments 58-60, wherein pembrolizumab is not administered in the first 42-day cycle and is administered each cycle starting from the second 42-day cycle.

Embodiment 65. The method of embodiment 64, wherein the administration of SEA-CD40 in each 21-day cycle, and the administration of pembrolizumab in each 42-day cycle except the first cycle, are on the following days of their respective cycles, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10. Embodiment 66. The method of embodiment 64 or 65, wherein the administration of SEA-CD40 in each 21-day cycle, and the administration of pembrolizumab in each 42-day cycle except the first cycle, are on the following days of their respective cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 67. The method of any one of embodiments 58-66 wherein pembrolizumab is administered intravenously.

Embodiment 68. The method of any one of embodiments 58-67 wherein pembrolizumab is administered at a dose of 400 mg.

Embodiment 69. The method of any one of embodiments 58-68, wherein pemetrexed is administered at a dose of 500 mg per square meter.

Embodiment 70. The method of any one of embodiments 58-69, wherein carboplatin is administered at a dose of area under the concentration-time curve (AUC) 2-8 mg per milliliter per minute.

Embodiment 71. The method of any one of embodiments 58-70, wherein carboplatin is administered at a dose of area under the concentration-time curve (AUC) 2 mg, 3 mg, 4 mg, 5 mg, 6 mg, 7 mg, or 8 mg per milliliter per minute.

Embodiment 72. The method of any one of embodiments 58-71, wherein carboplatin is administered at 5 mg per milliliter per minute.

Embodiment 73. The method of any one of embodiments 58-72 wherein the level of PD-L1 in a sample of the non-squamous NSCLC is less than 50% as measured by Total Proportion Score (TPS).

Embodiment 74. The method of any one of embodiments embodiment 58-73 wherein the level of PD-L1 in a sample of the non-squamous NSCLC is less than 1% as measured by TPS.

Embodiment 75. The method of any one of embodiments embodiment 58-73 wherein the level of PD-L1 in a sample of the non-squamous NSCLC is 1-49% as measured by TPS.

Embodiment 76. The method of any one of the embodiments 58-75 further comprising administering gemcitabine, dacarbazine, temozolomide, paclitaxel, and/or albumin-bound paclitaxel.

Embodiment 77. The method of embodiment 76 wherein the albumin-bound paclitaxel is nab-paclitaxel.

Embodiment 78. A method of treating squamous NSCLC comprising administering to a patient having squamous NSCLC: 1) SEA-CD40, 2) pembrolizumab, 3) carboplatin, and 4) paclitaxel or nab-paclitaxel.

Embodiment 79. The method of embodiment 78 wherein SEA-CD40, pembrolizumab, carboplatin and paclitaxel or nab-paclitaxel are all administered in 21-day cycles.

Embodiment 80. The method of embodiment 79 wherein carboplatin is administered on day 1 of the first four cycles (cycles 1-4) and not administered in the following cycles.

Embodiment 81. The method of embodiment 79 or 80 wherein paclitaxel, when administered, is administered on day 1 of each 21-day cycle at a dose of 200 mg per square meter of body-surface area; and nab-paclitaxel, when administered, is administered on days 1, 8, and 15 at a dose of 100 mg per square meter.

Embodiment 82. The method of any one of embodiments 79-81 wherein SEA-CD40 and pembrolizumab are administered on the following days of each 21-day cycle, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 83. The method of any one of embodiments 79-82, wherein SEA-CD40 and pembrolizumab are administered on the following days of each 21-day cycle, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 84. The method of any one of embodiments 79-82, wherein SEA-CD40 is administered on day 3 of each 21-day cycle, and pembrolizumab is administered on day 1 of each 21-day cycle.

Embodiment 85. The method of any one of embodiments 79-81, wherein pembrolizumab is not administered in the first 21-day cycle and is administered each cycle starting from the second 21-day cycle.

Embodiment 86. The method of embodiment 85, wherein the administration of SEA-CD40 in each 21-day cycle, and the administration of pembrolizumab in each 21-day cycle except the first cycle, are on the following days of the 21-day cycles, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 87. The method of embodiment 85 or 86, wherein the administration of SEA-CD40 in each 21-day cycle, and the administration of pembrolizumab in each 21-day cycle except the first cycle, are on the following days of the 21-day cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 88. The method of any one of embodiments 79-87 wherein pembrolizumab is administered intravenously.

Embodiment 89. The method of any one of embodiments 79-88 wherein pembrolizumab is administered at a dose of 200 mg.

Embodiment 90. The method of any one of embodiments 79-89, wherein carboplatin is administered at a dose of area under the concentration-time curve (AUC) 2-8 mg per milliliter per minute.

Embodiment 91. The method of any one of embodiments 79-90, wherein carboplatin is administered at a dose of area under the concentration-time curve (AUC) 2 mg, 3 mg, 4 mg, 5 mg, 6 mg, 7 mg, or 8 mg per milliliter per minute.

Embodiment 92. The method of any one of embodiments 79-91, wherein carboplatin is administered at 6 mg per milliliter per minute.

Embodiment 93. The method of any one of embodiments 79-92 wherein the level of PD-L1 in a sample of the squamous NSCLC is less than 50% as measured by Total Proportion Score (TPS).

Embodiment 94. The method of any one of embodiments embodiment 79-93 wherein the level of PD-L1 in a sample of the squamous NSCLC is less than 1% as measured by TPS.

Embodiment 95. The method of any one of embodiments embodiment 79-93 wherein the level of PD-L1 in a sample of the squamous NSCLC is 1-49% as measured by TPS.

Embodiment 96. The method of any one of the embodiments 79-95 further comprising administering gemcitabine, dacarbazine, temozolomide, or pemetrexed.

Embodiment 97. A method of treating squamous NSCLC comprising administering to a patient having squamous NSCLC: 1) SEA-CD40, 2) pembrolizumab, 3) carboplatin, and 4) paclitaxel or nab-paclitaxel, wherein SEA-CD40, carboplatin and paclitaxel or nab-paclitaxel are administered in 21-day cycles and pembrolizumab is administered in 42-day cycles. Embodiment 98. The method of embodiment 97 wherein carboplatin is administered on day 1 of the first four cycles (cycles 1-4) and not administered in the following cycles.

Embodiment 99. The method of embodiment 97 or 98 wherein paclitaxel, when administered, is administered on day 1 of each 21-day cycle at a dose of 200 mg per square meter of body-surface area; and nab-paclitaxel, when administered, is administered on days 1, 8, and 15 at a dose of 100 mg per square meter.

Embodiment 100. The method of any one of embodiments 97-99 wherein SEA-CD40 and pembrolizumab are administered on the following days of their respective cycles, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 101. The method of any one of embodiments 97-100, wherein SEA-CD40 and pembrolizumab are administered on the following days of their respective cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 102. The method of any one of embodiments 97-100, wherein SEA-CD40 is administered on day 3 of each 21-day cycle, and pembrolizumab is administered on day 1 of each 242-day cycle.

Embodiment 103. The method of any one of embodiments 97-99, wherein pembrolizumab is not administered in the first 42-day cycle and is administered each cycle starting from the second 42-day cycle.

Embodiment 104. The method of embodiment 103, wherein the administration of SEA-CD40 in each 21-day cycle, and the administration of pembrolizumab in each 42-day cycle except the first cycle, are on the following days of their respective cycles, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10.

Embodiment 105. The method of embodiment 103 or 104, wherein the administration of SEA-CD40 in each 21-day cycle, and the administration of pembrolizumab in each 42-day cycle except the first cycle, are on the following days of their respective cycles, respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 3 and day 4, day 3 and day 5, day 3 and day 6, day 3 and day 7, day 3 and day 8, day 3 and day 9, day 3 and day 10, day 4 and day 5, day 4 and day 6, day 4 and day 7, day 4 and day 8, day 4 and day 9, day 4 and day 10, day 5 and day 6, day 5 and day 7, day 5 and day 8, day 5 and day 9, or day 5 and day 10.

Embodiment 106. The method of any one of embodiments 97-105 wherein pembrolizumab is administered intravenously.

Embodiment 107. The method of any one of embodiments 97-106 wherein pembrolizumab is administered at a dose of 400 mg.

Embodiment 108. The method of any one of embodiments 97-107, wherein carboplatin is administered at a dose of area under the concentration-time curve (AUC) 2-8 mg per milliliter per minute.

Embodiment 109. The method of any one of embodiments 97-108, wherein carboplatin is administered at a dose of area under the concentration-time curve (AUC) 2 mg, 3 mg, 4 mg, 5 mg, 6 mg, 7 mg, or 8 mg per milliliter per minute.

Embodiment 110. The method of any one of embodiments 97-109, wherein carboplatin is administered at 6 mg per milliliter per minute.

Embodiment 111. The method of any one of embodiments 97-110 wherein the level of PD-L1 in a sample of the squamous NSCLC is less than 50% as measured by Total Proportion Score (TPS).

Embodiment 112. The method of any one of embodiments embodiment 97-111 wherein the level of PD-L1 in a sample of the squamous NSCLC is less than 1% as measured by TPS.

Embodiment 113. The method of any one of embodiments embodiment 97-111 wherein the level of PD-L1 in a sample of the squamous NSCLC is 1-49% as measured by TPS.

Embodiment 114. The method of any one of the embodiments 97-113 further comprising administering gemcitabine, dacarbazine, temozolomide, or pemetrexed.

Embodiment 115. The method of any one of embodiments 1-114, wherein SEA-CD40 comprises a heavy chain variable region comprising amino acid 1-113 of SEQ ID NO: 1, and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue.

Embodiment 116. The method of embodiment 115 wherein less than 10% of N-glycoside-linked sugar chains in the composition comprise a fucose residue.

Embodiment 117. The method of embodiment 115 or 116 wherein less than 5% of N-glycoside-linked sugar chains in the composition comprise a fucose residue.

Embodiment 118. The method of any one of embodiments 115-117 wherein less than 3% of N-glycoside-linked sugar chains in the composition comprise a fucose residue.

Embodiment 119. The method of any one of embodiments 115-118 wherein less than 2% of N-glycoside-linked sugar chains in the composition comprise a fucose residue.

Embodiment 120. The method of any one of embodiments 115-119 wherein the anti-CD40 antibody comprises a heavy chain comprising the amino acid sequence of SEQ ID NO: 1 and a light chain comprising the amino acid sequence of SEQ ID NO: 2.

Embodiment 121. The method of any one of embodiments 1-120, wherein pembrolizumab comprises a light chain comprising CDRs of SEQ ID NOs: 3-5, and a heavy chain comprising CDRs of SEQ ID NOs 8-10.

Embodiment 122. The method of any one of embodiments 1-121 wherein pembrolizumab comprises a light chain variable region comprising the amino acid sequence of SEQ ID NO: 6, and a heavy chain variable region comprising the amino acid sequence of SEQ ID NO: 11.

Embodiment 123. The method of any one of embodiments 1-122 wherein pembrolizumab comprises a light chain comprising the amino acid sequence of SEQ ID NO: 7, and a heavy chain comprising the amino acid sequence of SEQ ID NO: 12.

Embodiment 124. The method of any one of embodiments 1-123 wherein SEA-CD40 is administered intravenously.

Embodiment 125. The method of any one of embodiments 1-124 wherein SEA-CD40 is administered at a dose of 10 μg/kg.

Embodiment 126. The method of any one of embodiments 1-124 wherein SEA-CD40 is administered at a dose of 30 μg/kg.

### Definitions

The term “combination therapy” or “combination” refers to a treatment regimen including administering more than one therapeutic agent. A combination therapy can include two, three, four, five six, seven, eight, night, ten or a number of therapeutic agents. Each therapeutic agent can be the same or different kind of molecule including, e.g., a biologic agent, a small molecule, an antibody, a chemotherapeutic agent, etc. Each therapeutic agent can be administered in the same or different cycles. Some or all of the therapeutic agents can be formulated together. Some or all of the therapeutic agents can be administered separately.

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but can be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “antibody” is used herein to denote immunoglobulin proteins produced by the body in response to the presence of an antigen and that bind to the antigen, as well as antigen-binding fragments and engineered variants thereof. Hence, the term “antibody” includes, for example, intact monoclonal antibodies comprising full-length immunoglobulin heavy and light chains (e.g., antibodies produced using hybridoma technology) and antigen-binding antibody fragments, such as F(ab′)2 and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific (e.g., bispecific) hybrid antibodies, and the like are also included. Thus, the term “antibody” is used expansively to include any protein that comprises an antigen-binding site of an antibody and is capable of specifically binding to its antigen.

An “antigen-binding site of an antibody” is that portion of an antibody that is sufficient to bind to its antigen. The minimum such region is typically a variable region or a genetically engineered variant thereof. Single-domain binding sites can be generated from camelid antibodies (see Muyldermans and Lauwereys, J. Mol. Recog. 12.131-140, 1999; Nguyen et al., EMBO J. 19:921-930, 2000) or from VH domains of other species to produce single-domain antibodies (“dAbs”; see Ward et al., Nature 341:544-546, 1989; U.S. Pat. No. 6,248,516 to Winter et al.). In certain variations, an antigen-binding site is a polypeptide region having only 2 complementarity determining regions (CDRs) of a naturally or non-naturally (e.g., mutagenized) occurring heavy chain variable region or light chain variable region, or combination thereof (see, e.g., Pessi et al., Nature 362:367-369, 1993; Qiu et al., Nature Biotechnol. 25:921-929, 2007). More commonly, an antigen-binding site of an antibody comprises both a heavy chain variable (VH) domain and a light chain variable (VL) domain that bind to a common epitope. Within the context of the present disclosure, an antibody can include one or more components in addition to an antigen-binding site, such as, for example, a second antigen-binding site of an antibody (which can bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBSLetters 450:23-26, 1999), a cytostatic or cytotoxic drug, and the like, and can be a monomeric or multimeric protein. Examples of molecules comprising an antigen-binding site of an antibody are known in the art and include, for example, Fv, single-chain Fv (scFv), Fab, Fab′, F(ab′)₂, F(ab)_{c}, diabodies, dAbs, minibodies, nanobodies, Fab-scFv fusions, bispecific (scFv)₄-IgG, and bispecific (scFv)₂-Fab. (See, e.g., Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002.)

The terms “cancer”, “cancerous”, or “malignant” refer to or describe the physiological condition in mammals that is typically characterized by unregulated cell growth. The cancer can be a solid tumor or a blood cancer. The cancer can also be a melanoma, a breast cancer, including metastatic breast cancer, a lung cancer, including a non-small cell lung cancer, pancreatic cancer, lymphoma, colorectal cancer, or renal cancer. In some embodiments, the cancer is a melanoma; a breast cancer, including metastatic breast cancer; a lung cancer, including a non-small cell lung cancer; or pancreatic cancer. The pancreatic cancer can be a pancreatic ductal adenocarcinoma (PDAC). The PDAC can also be metastatic.

As used herein, the term “immunoglobulin” refers to a protein consisting of one or more polypeptides substantially encoded by immunoglobulin gene(s). One form of immunoglobulin constitutes the basic structural unit of native (i.e., natural) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol 7. For example, immunoglobulins of the IgG1 subclass contain a 71 heavy chain constant region. Each immunoglobulin heavy chain possesses a constant region that consists of constant region protein domains (CH1, hinge, CH2, and CH3; IgG3 also contains a CH4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function, see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable region” (“VL region”) or “heavy chain variable region” (“VH region”), respectively) consists of a “framework” region interrupted by three hypervariable regions, also called “complementarity determining regions” or “CDRs.” The framework regions serve to align the CDRs for specific binding to an epitope of an antigen. Thus, the term “hypervariable region” or “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, MD, 1987 and 1991), or Chothia & Lesk, J. Mol. Biol. 196:901-917, 1987; Chothia et al., Nature 342:878-883, 1989. Kabat also provides a widely used numbering convention (Kabat numbering) in which corresponding residues between different heavy chains or between different light chains are assigned the same number. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3.

Unless the context dictates otherwise, the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The term “chimeric antibody” refers to an antibody having variable regions derived from a first species and constant regions derived from a second species. Chimeric immunoglobulins or antibodies can be constructed, for example by genetic engineering, from immunoglobulin gene segments belonging to different species. The term “humanized antibody,” as defined infra, is not intended to encompass chimeric antibodies. Although humanized antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized VH domain” or “humanized VL domain” refers to an immunoglobulin VH or VL domain comprising some or all CDRs entirely or substantially from a non-human donor immunoglobulin (e.g., a mouse or rat) and variable region framework sequences entirely or substantially from human immunoglobulin sequences. The non-human immunoglobulin providing the CDRs is called the “donor” and the human immunoglobulin providing the framework is called the “acceptor.” In some instances, humanized antibodies can retain non-human residues within the human variable framework regions to enhance proper binding characteristics (e.g., mutations in the frameworks can be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

Specific binding of an antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that a monoclonal antibody binds one and only one target.

With regard to proteins as described herein, reference to amino acid residues corresponding to those specified by SEQ ID NO includes post-translational modifications of such residues.

The term “diluent” as used herein refers to a solution suitable for altering or achieving an exemplary or appropriate concentration or concentrations as described herein.

The term “container” refers to something into which an object or liquid can be placed or contained, e.g., for storage (for example, a holder, receptacle, vessel, or the like).

The term “administration route” includes art-recognized administration routes for delivering a therapeutic protein such as, for example, parenterally, intravenously, intramuscularly, or subcutaneously. For administration of an antibody for the treatment of cancer, administration into the systemic circulation by intravenous or subcutaneous administration can be desired. For treatment of a cancer characterized by a solid tumor, administration can also be localized directly into the tumor, if so desired.

The term “treatment” refers to the administration of a therapeutic agent to a patient, who has a disease with the purpose to cure, heal, alleviate, delay, relieve, alter, remedy, ameliorate, improve or affect the disease.

The term “paclitaxel” refers to the chemotherapeutic agent paclitaxel in original form or various formulations such as “albumin-bound paclitaxel” and “ABRAXANE©” (nab-paclitaxel) which is a brand name of paclitaxel containing albumin-bound paclitaxel.

The term “patient” includes human and other mammalian subjects that receive either prophylactic or therapeutic treatment.

The term “effective amount,” “effective dose,” or “effective dosage” refers to an amount that is sufficient to achieve or at least partially achieve the desired effect, e.g., sufficient to inhibit the occurrence or ameliorate one or more symptoms of a disease or disorder. An effective amount of a pharmaceutical composition is administered in an “effective regime.” The term “effective regime” refers to a combination of amount of the composition being administered and dosage frequency adequate to accomplish prophylactic or therapeutic treatment of the disease or disorder.

As used herein, the term “about” denotes an approximate range of plus or minus 10% from a specified value. For instance, the language “about 20 μg/kg” encompasses a range of 18-22 μg/kg. As used herein, about also includes the exact amount. Hence “about 20 μg/kg” means “about 20 μg/kg” and also “20 μg/kg.”

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR (framework) regions of the variable regions or located in the constant regions, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“PD-1 antagonist” means any chemical compound or biological molecule that blocks binding of PD-L1 (e.g., expressed on a cancer cell) to PD-1 (e.g., expressed on an immune cell (T cell, B cell or NKT cell)) and preferably also blocks binding of PD-L2 (e.g., expressed on a cancer cell) to PD-1 (e.g., immune-cell expressed PD-1). Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCDIL2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L 1 and PD-L2 amino acid sequences can be found in NCBI Locus No.: NP_054862 and NP_079515, respectively.

As used herein, a “SEA-CD40 variant” means a monoclonal antibody which comprises heavy chain and light chain sequences that are substantially identical to those in SEA-CD40, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR (framework) regions of the variable regions or located in the constant regions, and optionally has a deletion of the C-terminal lysine residue of the heavy chain. In other words, SEA-CD40 and a SEA-CD40 variant comprise identical CDR sequences, but differ from each other due to having a conservative amino acid substitution at no more than three or six other positions in their full length light and heavy chain sequences, respectively. A SEA-CD40 variant is substantially the same as SEA-CD40 with respect to the following properties: binding affinity to CD40 and non-fucosylation characteristics.

“Conservatively amino acid substitutions” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr, Phe |
| Tyr (Y) | Trp; Phe |
| Val (V) | Ile; Leu |

“Combined Positive Score” or “CPS” is an immunohistochemical method of measuring PD-L1 expression in a cancer, such as a tumor sample from a cancer. CPS is the number of PD-L1 staining cells (tumor cells, lymphocytes, macrophages) divided by the total number of viable tumor cells, multiplied by 100. For some therapeutic treatments, a tumor sample is considered to have PD-L1 expression if CPS≥1. For example, a CPS≥1 is required for a subject to be eligible for certain PD-1 or PD-L1 inhibitor therapies, such as subjects with gastric cancer, cervical cancer, and head and neck squamous cell cancer. In some instances, a CPS≥10 is required for a subject to be eligible for certain PD-1 or PD-L1 inhibitor therapies, such as subjects with urothelial cancer (bladder cancer), esophageal squamous cell carcinoma (ESCC), or triple-negative breast cancer being treated with pembrolizumab.

“Tumor Proportion Score” or “TPS” is an immunohistochemical method of measuring PD-L1 expression in a cancer, such as a tumor sample from a cancer. TPS is the percentage of viable tumor cells showing partial or complete membrane staining at any intensity. For some therapeutic treatments, a tumor sample is considered to have PD-L1 expression if TPS≥1% and high PD-L1 expression if TPS≥50%. For example, a TPS 1% is the required for a subject to be eligible for certain PD-1 or PD-L1 inhibitor therapies (e.g., pembrolizumab), such as subjects with non-small cell lung cancer. In some instances, a TPS≥50% is the required for a subject to be eligible for certain PD-1 or PD-L1 inhibitor therapies (e.g., cemiplimab).

Tumor-Infiltrating Immune Cell (IC) staining or “IC” is an immunohistochemical method of measuring PD-L1 expression, such as a tumor sample from a cancer. The expression is measured as the proportion of tumor area that is occupied by PD-L1 staining IC of any intensity. If the specimen contains PD-L1 staining of any intensity in tumor infiltrating immune cells occupying≥5% of tumor area, then the specimen is assigned a PD-L1 expression level of ≥5% IC. If the specimen contains PD-L1 staining of any intensity in tumor-infiltrating immune cells covering<5% of tumor area, then the specimen is assigned a PD-L1 expression level of <5% IC. For some therapeutic treatments, IC is used to score PD-L1 expression from urothelial carcinoma tissue. Urothelial carcinoma tissue samples obtained from resections, transurethral resection of bladder tumor (TURBT), and core needle biopsies from both primary and metastatic sites can be used in IC assays. Commercially available IC assays include the Ventana PD-L1 (SP142) Assay™

A Tumor Cell or “TC” score refers to the percentage of PD-L1 expressing tumor cells (% TC) of any intensity, and is similar to TPS. In some embodiments, a TC score is obtained using the Ventana PD-L1 (SP142) Assay. TC scores are used, for example, when NSCLC patients are treated with atezolizumab (TECENTRIQ). In this indication, the threshold for treatment is a TC score of ≥50%. Further information on TC scoring is available, for example, in. 1) Physician Labeling: Ventana PD-L1 (SP142) Assay (2020) Ventana Medical Systems, Inc. and Roche Diagnostics International, Inc.; and 2) Ventana PD-L1 (SP142) Assay: Interpretation Guide (2019) Ventana Medical Systems, Inc. and Roche Diagnostics International, Inc.

A patient who has “PD-(L)1 naïve melanoma” or “metastatic PD-(L)1 naïve melanoma” means that the patient must not have received prior treatment for advanced or metastatic disease except for prior adjuvant/neoadjuvant immunotherapy such as interferon, anti-PD-1, anti-PD-L1, or anti-CTLA-4, and the patient does not have uveal melanoma or ocular melanoma. If the patient has BRAF mutation, the patient is still considered to have “PD-(L)1 naïve melanoma” or “metastatic PD-(L)1 naïve melanoma” if prior BRAF/MEK targeted therapy was completed 4 weeks prior to first dose of a combination therapy described therein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1A shows the median tumor volume of mice of a CT26 colon cancer model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by simultaneous dosing (G4; “SEA-m1C10+anti-PD1”).

FIG. 1B shows the median tumor volume of mice of a CT26 colon cancer model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by staggered dosing (G4; “SEA-m1C10+anti-PD1”). The time period for SEA-m1C10 dosing and anti-PD1 dosing are labeled by vertical lines on the X-axis.

FIG. 2A shows the survival curves of mice of a A20 disseminated lymphoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by simultaneous dosing (G4; “SEA-m1C10+anti-PD1”).

FIG. 2B shows the mean tumor volume of mice of a A20 disseminated lymphoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by staggered dosing (G4; “SEA-m1C10+anti-PD1”). The time period for SEA-m1C10 dosing and anti-PD1 dosing are labeled by vertical lines on the X-axis.

FIG. 3A shows the mean tumor volume of mice of a RENCA renal cell carcinoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by simultaneous dosing (G4; “SEA-m1C10+anti-PD1”).

FIG. 3B shows the mean tumor volume of mice of a RENCA renal cell carcinoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by staggered dosing (G4; “SEA-m1C10+anti-PD1”). The time period for SEA-m1C10 dosing and anti-PD1 dosing are labeled by vertical lines on the X-axis.

FIG. 4 lists sequences discussed in the disclosure. Variable regions are in bold and underlined.

### SEQUENCE LISTING

This application contains a Sequence Listing that has been submitted electronically as an XML file named 492230059WO1.XML. The XML filed, created on Jul. 27, 2022, is 12,868 bytes in size. The material in the XML file is hereby incorporated by reference in its entirety.

### DETAILED DESCRIPTION

This disclosure relates to methods of treating melanoma using a combination of an anti-CD40 antibody such as SEA-CD40 or a SEA-CD40 variant, and an anti-PD-1 antibody such as pembrolizumab or a pembrolizumab variant. This disclosure also relates to methods of treating non-small cell lung cancer (NSCLC) using a combination of an anti-CD40 antibody such as SEA-CD40 or a SEA-CD40 variant, and an anti-PD-1 antibody such as pembrolizumab or a pembrolizumab variant, and chemotherapy.

### CD40

CD40 is a member of the tumor necrosis factor (TNF) receptor superfamily. It is a single chain type I transmembrane protein with an apparent MW of 50 kDa. Its mature polypeptide core consists of 237 amino acids, of which 173 amino acids comprise an extracellular domain (ECD) organized into 4 cysteine-rich repeats that are characteristic of TNF receptor family members. Two potential N-linked glycosylation sites are present in the membrane proximal region of the ECD, while potential O-linked glycosylation sites are absent. A 22 amino acid transmembrane domain connects the ECD with the 42 amino acid cytoplasmic tail of CD40. Sequence motifs involved in CD40-mediated signal transduction have been identified in the CD40 cytoplasmic tail. These motifs interact with cytoplasmic factors called TNF-R-associated factors (TRAFs) to trigger multiple downstream events including activation of MAP kinases and NFκB, which in turn modulate the transcriptional activities of a variety of inflammation-, survival-, and growth-related genes. See, e.g., van Kooten and Banchereau, J. Leukoc. Biol. 67:2-17 (2000); Elgueta et al., Immunol. Rev. 229:152-172 (2009).

Within the hematopoietic system, CD40 can be found on B cells at multiple stages of differentiation, monocytes, macrophages, platelets, follicular dendritic cells, dendritic cells (DC), eosinophils, and activated T cells. In normal non-hematopoietic tissues, CD40 has been detected on renal epithelial cells, keratinocytes, fibroblasts of synovial membrane and dermal origins, and activated endothelium. A soluble version of CD40 is released from CD40-expressing cells, possibly through differential splicing of the primary transcript or limited proteolysis by the metalloproteinase TNFα converting enzyme. Shed CD40 can potentially modify immune responses by interfering with the CD40/CD40L interaction. See, e.g., van Kooten and Banchereau, J. Leukoc. Biol. 67:2-17 (2000); Elgueta et al., Immunol. Rev. 229:152-172 (2009).

The endogenous ligand for CD40 (CD40L) is a type II membrane glycoprotein of 39 kDa also known as CD154. CD40L is a member of the TNF superfamily and is expressed as a trimer on the cell surface. CD40L is transiently expressed on activated CD4+, CD8+, and γδT cells. CD40L is also detected at variable levels on purified monocytes, activated B cells, epithelial and vascular endothelial cells, smooth muscle cells, and DCs, but the functional relevance of CD40L expression on these cell types has not been clearly defined (van Kooten 2000; Elgueta 2009). However, expression of CD40L on activated platelets has been implicated in the pathogenesis of thrombotic diseases. See, e.g., Ferroni et al., Curr. Med. Chem. 14:2170-2180 (2007).

The best-characterized function of the CD40/CD40L interaction is its role in contact-dependent reciprocal interaction between antigen-presenting cells and T cells. See, e.g., van Kooten and Banchereau, J. Leukoc. Biol. 67:2-17 (2000); Elgueta et al., Immunol. Rev. 229:152-172 (2009). Binding of CD40L on activated T cells to CD40 on antigen-activated B cells not only drives rapid B cell expansion, but also provides an essential signal for B cells to differentiate into either memory B cells or plasma cells. CD40 signaling is responsible for the formation of germinal centers in which B cells undergo affinity maturation and isotype switching to acquire the ability to produce high affinity antibodies of the IgG, IgA, and IgE isotypes. See, e.g., Kehry, J. Immunol. 156:2345-2348 (1996). Thus, individuals with mutations in the CD40L locus that prevent functional CD40/CD40L interaction suffer from the primary immunodeficiency X-linked hyper-IgM syndrome that is characterized by over-representation of circulating IgM and the inability to produce IgG, IgA, and IgE. These patients demonstrate suppressed secondary humoral immune responses, increased susceptibility to recurrent pyrogenic infections, and a higher frequency of carcinomas and lymphomas. Gene knockout experiments in mice to inactivate either CD40 or CD40L locus reproduce the major defects seen in X-linked hyper-IgM patients. These KO mice also show impaired antigen-specific T cell priming, suggesting that the CD40L/CD40 interaction is also a critical factor for mounting cell-mediated immune responses. See, e.g., Elgueta et al., Immunol. Rev. 229:152-172 (2009).

The immune-stimulatory effects of CD40 ligation by CD40L or anti-CD40 in vivo have correlated with immune responses against syngeneic tumors. See, e.g., French et al., Nat. Med. 5:548-553 (1999). A deficient immune response against tumor cells can result from a combination of factors such as expression of immune checkpoint molecules, such as PD1 or CTLA-4, decreased expression of MHC antigens, poor expression of tumor-associated antigens, appropriate adhesion, or co-stimulatory molecules, and the production of immunosuppressive proteins like TGFβ by the tumor cells. CD40 ligation on antigen presenting and transformed cells results in up-regulation of adhesion proteins (e.g., CD54), co-stimulatory molecules (e.g., CD86) and MHC antigens, as well as inflammatory cytokine secretion, thereby potentially inducing and/or enhancing the antitumor immune response, as well as the immunogenicity of the tumor cells. See, e.g., Gajewski et al., Nat. Immunol. 14:1014-1022 (2013).

A primary consequence of CD40 cross-linking is DC activation (often termed licensing) and potentiation of myeloid and B cells ability to process and present tumor-associated antigens to T cells. Besides having a direct ability to activate the innate immune response, a unique consequence of CD40 signaling is APC presentation of tumor-derived antigens to CD8+ cytotoxic T cell (CTL) precursors in a process known as ‘cross-priming’. This CD40-dependent activation and differentiation of CTL precursors by mature DCs into tumor-specific effector CTLs can enhance cell-mediated immune responses against tumor cells. See, e.g., Kurts et al., Nat. Rev. Immunol. 10:403-414 (2010).

Agonistic CD40 mAbs including dacetuzumab, the SEA-CD40 parent molecule (a fucosylated anti-CD40 antibody), have shown encouraging clinical activity in single-agent and combination chemotherapy settings. Dacetuzumab demonstrated some clinical activity in a phase 1 study in NHL and a phase 2 study in diffuse large B-cell lymphoma (DLBCL). See, e.g., Advani et al., J. Clin. Oncol. 27:4371-4377 (2009) and De Vos et al., J. Hematol. Oncol. 7:1-9 (2014). Additionally CP-870,893, a humanized IgG2 agonist antibody to CD40, showed encouraging activity in solid tumor indications when combined with paclitaxel or carboplatin or gemcitabine. In these studies, activation of antigen presenting cells, cytokine production, and generation of antigen-specific T cells were seen. See, e.g., Beatty et al., Clin. Cancer Res. 19:6286-6295 (2013) and Vonderheide et al., Oncoimmunology 2:e23033 (2013)

### Anti-CD40 Antibodies

Anti-CD40 antibodies, e.g., S2C6, have been disclosed in US20170333556A1, which is herein incorporated by reference. The S2C6 antibody is a partial agonist of the CD40 signaling pathway and thus has the following activities: binding to human CD40 protein, binding to cynomolgus CD40 protein, activation of the CD40 signaling pathway, potentiation of the interaction of CD40 with its ligand, CD40L. See, e.g., U.S. Pat. No. 6,946,129, which is herein incorporated by reference.

Humanized anti-CD40 antibodies, e.g., humanized S2C6 (hS2C6), have been disclosed in U.S. Pat. No. 8,303,955B2 and U.S. Pat. No. 8,492,531B2, both of which are herein incorporated by reference.

Non-fucosylated anti-CD40 antibodies, e.g., hS2C6 or SEA-CD40 have been disclosed in US20170333556A1. In addition to enhanced binding to Fc receptors, SEA-CD40 also enhances activity of the CD40 pathway, as compared to the parent antibody, dacetuzumab. The SEA-CD40 antibody thus, is administered to patients at lower doses and using different schedules of administration.

SEA-CD40 exhibits enhanced binding to FcγIII receptors, and enhanced ability to activate the CD40 signaling pathway in immune cells, as described in US20170333556A1. Methods of making the non-fucosylated antibodies including SEA-CD40 are also disclosed in US20170333556A1.

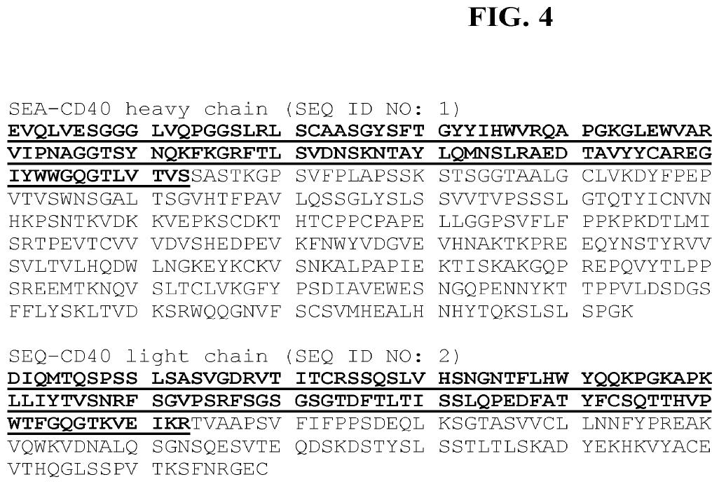

The amino acid sequences of the heavy chain and light chain for SEA-CD40 are disclosed as SEQ ID NOs: 1 and 2, respectively (See FIG. 4). As disclosed in US20170333556A1, the variable region of the heavy chain is from amino acids 1-113 of SEQ ID NO: 1. The variable region of the light chain is from amino acids 1-113 of SEQ ID NO: 2.

In some embodiment, a humanized anti-CD40 antibody disclosed herein is useful in the treatment of various disorders associated with the expression of CD40 as described herein. Because SEA-CD40 activates the immune system to respond against tumor-related antigens, its use is not limited to cancers that express CD40. Thus SEA-CD40 can be used to treat both CD40 positive and CD40 negative cancers.

### Methods of Making Non-Fucosylated Antibodies

This disclosure provides compositions and methods for preparing humanized S2C6 antibodies with reduced core fucosylation. As used herein, “core fucosylation” refers to addition of fucose (“fucosylation”) to N-acetylglucosamine (“GlcNAc”) at the reducing terminal of an N-linked glycan.

Fucosylation of complex N-glycoside-linked sugar chains bound to the Fc region (or domain) of the SEA-CD40 antibody backbone is reduced. As used herein, a “complex N-glycoside-linked sugar chain” is typically bound to asparagine 297 (according to the EU index as set forth in Kabat, “Sequences of Immunological Interest, 5^{th} Ed., Pub. No. 91-3242, U.S. Dept. Health & Human Services, NIH, Bethesda, MD, 1991). As used herein, the complex N-glycoside-linked sugar chain has a biantennary composite sugar chain, mainly having the following structure:

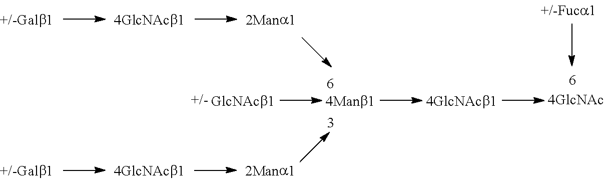

where ± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to the present methods, typically only a minor amount of fucose is incorporated into the complex N-glycoside-linked sugar chain(s) of the SEA-CD40 molecule. For example, in various embodiments, less than about 60%, less than about 50%, less than about 40%, less than about 30%, less than about 20%, less than about 15%, less than about 10%, less than about 5%, or less than about 3% of the antibody has core fucosylation by fucose. In some embodiments, about 2% of the antibody has core fucosylation by fucose.

In certain embodiments, only a minor amount of a fucose analog (or a metabolite or product of the fucose analog) is incorporated into the complex N-glycoside-linked sugar chain(s). For example, in various embodiments, less than about 40%, less than about 30%, less than about 20%, less than about 15%, less than about 10%, less than about 5%, or less than about 3% of the SEA-CD40 antibody has core fucosylation by a fucose analog or a metabolite or product of the fucose analog. In some embodiments, about 2% of the SEA-CD40 antibody has core fucosylation by a fucose analog or a metabolite or product of the fucose analog.

Methods of making non-fucosylated antibodies by incubating antibody-producing cells with a fucose analogue are described, e.g., in WO/2009/135181. Briefly, cells that have been engineered to express the humanized S2C6 antibody are incubated in the presence of a fucose analogue or an intracellular metabolite or product of the fucose analog. As used herein, an intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In some embodiments, the fucose analogue is 2-flurofucose. Methods of using fucose analogues in growth medium and other fucose analogues are disclosed, e.g., in WO/2009/135181, which is herein incorporated by reference.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that can produce a non-fucosylated antibody, e.g., an SEA-CD40 antibody.

Those of skill will recognize that many methods are available to determine the amount of fucosylation on an antibody. Methods include, e.g., LC-MS via PLRP-S chromatography and electrospray ionization quadrupole TOF MS.

The non-fucosylated antibody, SEA-CD40, when adminstered to a patient induces activation of monocyte maturation into macrophages and induce production of cytokines, including, e.g., interferon-γ (IFN-γ) and chemokine that elicit robust T-cell response to immune system challenges. Unlike fully agoninstic antibodies, such as antibody 24.4.1., SEA-CD40 does not induce production of immune-dampening cytokines, such as interleukin-10 (IL-10). IL-10, in turn, induces activity of T-regulatory cells, which dampen the immune response. Thus, SEA-CD40 is useful for induction of a robust T-cell mediated immune response without promoting activity of T-regulatory cells.

In some embodiments, the disclosure relates to a composition comprising a non-fucosylated anti-CD40 antibody such as SEA-CD40, wherein the constant region of the antibody such as SEA-CD40 has an N-glycoside-linked sugar chain at residue N297 according to the EU index; and wherein less than 40%, less than 30%, less than 20%, less than 15%, less than 10%, less than 5%, less than 3%, less than 2% of N-glycoside-linked sugar chains in the composition comprise a fucose residue. In some embodiments, less than 20% of N-glycoside-linked sugar chains in the composition comprise a fucose residue. In some embodiments, less than 10% of N-glycoside-linked sugar chains in the composition comprise a fucose residue. In some embodiments, less than 5% of N-glycoside-linked sugar chains in the composition comprise a fucose residue. In some embodiments, less than 3% of N-glycoside-linked sugar chains in the composition comprise a fucose residue. In some embodiments, less than 2% of N-glycoside-linked sugar chains in the composition comprise a fucose residue.

In some embodiments, the disclosure relates to treating a cancer by administering a composition comprising a non-fucosylated anti-CD40 antibody such as SEA-CD40, wherein the constant region of the antibody such as SEA-CD40 has an N-glycoside-linked sugar chain at residue N297 according to the EU index; and wherein less than 40%, less than 30%, less than 20%, less than 15%, less than 10%, less than 5%, less than 3%, less than 2% of N-glycoside-linked sugar chains in the composition comprise a fucose residue. In some embodiments, less than 20% of N-glycoside-linked sugar chains in the composition comprise a fucose residue. In some embodiments, less than 10% of N-glycoside-linked sugar chains in the composition comprise a fucose residue. In some embodiments, less than 5% of N-glycoside-linked sugar chains in the composition comprise a fucose residue. In some embodiments, less than 3% of N-glycoside-linked sugar chains in the composition comprise a fucose residue. In some embodiments, less than 2% of N-glycoside-linked sugar chains in the composition comprise a fucose residue.

The humanized anti-CD40 antibody or agent is administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). The humanized anti-CD40 antibody or agent can be administered, for example, as an infusion or as a bolus. Parenteral infusions include intramuscular, intravenous, intraarterial, intraperitoneal, or subcutaneous administration. In addition, the humanized anti-CD40 antibody is suitably administered by pulse infusion, particularly with declining doses of the antibody. In one aspect, the dosing is given by injections, most preferably intravenous or subcutaneous injections, depending in part on whether the administration is brief or chronic.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat cancers including cancers. Because SEA-CD40 activates the immune system to respond against tumor-related antigens, its use is not limited to cancers that express CD40. Thus SEA-CD40 can be used to treat both CD40 positive and CD40 negative cancers.

The antibody need not be, but is optionally, formulated with one or more agents currently used to prevent or treat the disorder in question. The effective amount of such other agents depends on the amount of humanized anti-CD40 antibody present in the formulation, the type of disorder or treatment, and other factors discussed above. These are generally used in the same dosages and with administration routes as used hereinbefore or about from 1 to 99% of the heretofore employed dosages.

PD-1 antagonists useful in the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the treatment method, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist in the treatment method, medicaments and uses of the present invention include:
pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in Table 2; nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712, and AMP-514, which is being developed by MedImmune.

Examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MED14736, MSB0010718C and an antibody which comprises the heavy chain and light chain variable regions of SEQ ID NO: 24 and SEQ ID NO: 21, respectively, of WO2013/019906.

Other PD-1 antagonists useful in the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some preferred embodiments of the treatment method, medicaments and uses of the present invention, the PD-1 antagonist is a monoclonal antibody, or antigen binding fragment thereof, which comprises: (a) light chain CDRs SEQ ID NOs: 3, 4 and 5 and (b) heavy chain CDRs SEQ ID NOs: 6, 7 and 8.

In other preferred embodiments of the treatment method, medicaments and uses of the present invention, the PD-1 antagonist is a monoclonal antibody, or antigen binding fragment thereof, which specifically binds to human PD-1 and comprises (a) a heavy chain variable region comprising SEQ ID NO: 11 or a variant thereof, and (b) a light chain variable region comprising SEQ ID NO: 6 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the PD-1 antagonist is a monoclonal antibody which specifically binds to human PD-1 and comprises (a) a heavy chain comprising SEQ ID NO: 12 and (b) a light chain comprising SEQ ID NO: 7.

In all of the above treatment method, medicaments and uses, the PD-1 antagonist inhibits the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain, and wherein the heavy and light chains comprise the amino acid sequences in SEQ ID NO: 12 and SEQ ID NO: 7, respectively.

In one embodiment, the PD-1 antagonist is an anti-PD-1 antibody. In one embodiment, the anti-PD-1 antibody is pembrolizumab. In one embodiment, the anti-PD-1 antibody is a pembrolizumab variant.

Table 2 below provides a list of the amino acid sequences of exemplary anti-PD-1 mAbs for use in the treatment method, medicaments and uses of the present invention.

TABLE 2
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 3 |
| CDR2 | LASYLES | 4 |
| CDR3 | QHSRDLPLT | 5 |
| Variable | EIVLTQSPATLSLSPGERATLSCRASKGVSTSGYSYLHWY | 6 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 7 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 8 |
| CDR2 | GINPSNGGTNFNEKFKN | 9 |
| CDR3 | RDYRFDMGFDY | 10 |
| Variable | QVQLVQSGVEVKKPGASVKVSCKASGYTFTNYYMYWV | 11 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 12 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |

### Dosage and Administration of an Anti-CD40 Antibody Such as SEA-CD40 for Treating Cancer

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

In some embodiments, an anti-CD40 antibody such as SEA-CD40 is administered intravenously. In some embodiments, an anti-CD40 antibody such as SEA-CD40 is administered subcutaneously. In a further embodiment, an anti-CD40 antibody such as SEA-CD40 is administered subcutaneously at the site of a tumor.

SEA-CD40 is an agonistic antibody and has enhanced binding to Fcγ receptors III and, exhibits enhanced activation of the CD40 signaling pathway. Because of its enhanced activation of the CD40 pathway SEA-CD40 is a potent activator of the immune system. The enhanced activation of the immune system allows SEA-CD40 to be dosed at low levels, as compared to a fucosylated parent antibody.

As an example, an anti-CD40 antibody such as SEA-CD40 can be administered to patients at levels between about 0.1 to about 70 μg/kg (g antibody per kilogram patient body weight). Other possible dosage ranges include about 1 μg/kg to about 60 μg/kg, about 10 μg/kg to about 50 μg/kg, and about 20 μg/kg to about 40 μg/kg. Other possible dosage ranges include the following: about 1 μg/kg to about 5 μg/kg, about 5 μg/kg to about 10 μg/kg, about 10 μg/kg to about 15 μg/kg, about 15 μg/kg to about 20 μg/kg, about 20 μg/kg to about 25 μg/kg, about 25 μg/kg to about 30 μg/kg, about 30 μg/kg to about 35 μg/kg, about 35 μg/kg to about 40 μg/kg, about 40 μg/kg to about 45 μg/kg, about 45 μg/kg to about 50 μg/kg, about 50 μg/kg to about 55 μg/kg, and about 55 μg/kg to about 60 μg/kg.

In some embodiments, an anti-CD40 antibody such as SEA-CD40 is administered to patients at about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 ag/kg, about 54 μg/kg, about 55 μg/kg, about 56 μg/kg, about 57 μg/kg, about 58 μg/kg, about 59 ag/kg, about 60 μg/kg, about 61 μg/kg, about 62 μg/kg, about 63 μg/kg, about 64 μg/kg, about 65 ag/kg, about 66 μg/kg, about 67 μg/kg, about 68 μg/kg, about 69 μg/kg, or about 70 μg/kg. In preferred embodiments, an anti-CD40 antibody such as SEA-CD40 is administered to patients at about 3 μg/kg, about 10 μg/kg, about 30 μg/kg, about 45 μg/kg, or about 60 μg/kg. In a more preferred embodiment, an anti-CD40 antibody such as SEA-CD40 is administered to cancer patients at about 30 μg/kg or about 10 μg/kg. In another more preferred embodiment, an anti-CD40 antibody such as SEA-CD40 is administered to cancer patients at about 10 μg/kg. In yet another more preferred embodiment, an anti-CD40 antibody such as SEA-CD40 is administered to cancer patients at about 30 μg/kg.

An anti-CD40 antibody such as SEA-CD40 can be administered at different intervals including one week intervals, two weeks intervals, three week intervals, four weeks intervals, five week intervals, six week intervals, seven week intervals, eight week intervals, nine weeks, ten weeks, eleven weeks, twelve weeks, etc. In other words, an anti-CD40 antibody such as SEA-CD40 can be administered every week, every two weeks, every three weeks, every four weeks, every five weeks, every six weeks, every seven weeks, every eight weeks, every nine weeks, every ten weeks, every eleven weeks, every twelve weeks etc. In some embodiments, intervals are on a monthly schedule, e.g., one month intervals, two month intervals, or three month intervals. In some embodiments, intervals are based on cycles wherein each cycle can comprise one or more administrations of an anti-CD40 antibody such as SEA-CD40. Exemplary lengths of each cycle include one week, two weeks, three weeks, four weeks, five week, six weeks, seven weeks, eight weeks, nine weeks, ten weeks, eleven weeks, and twelve weeks. The lengths of cycles can be different from one cycle to the next. An anti-CD40 antibody such as SEA-CD40 can be administered on any one or more days in each cycle. In some embodiments, an anti-CD40 antibody such as SEA-CD40 is administered on the first day of a cycle. In some embodiments, an anti-CD40 antibody such as SEA-CD40 is administered on the first day of a cycle of three weeks in a treatment period of one cycle, two cycles, three cycles, four cycles, five cycles, six cycles, seven cycles, eight cycles, nine cycles, ten cycles, eleven cycles, twelve cycles, thirteen cycles, fourteen cycles, fifteen cycles, or sixteen cycles.

An anti-CD40 antibody such as SEA-CD40 can be administered on day 1, day 2, day 3, day 4, day 5, day 6, or day 7 of each 1-week cycle, i.e., an anti-CD40 antibody such as SEA-CD40 is administered every week starting on day 1, day 2, day 3, day 4, day 5, day 6, or day 7 of a treatment regimen. An anti-CD40 antibody such as SEA-CD40 can be administered on day 1, day 2, day 3, day 4, day 5, day 6, day 7, day 8, day 9, day 10, day 11, day 12, day 13, or day 14 of each 2-week cycle, i.e., an anti-CD40 antibody such as SEA-CD40 is administered every two weeks starting on day 1, day 2, day 3, day 4, day 5, day 6, day 7, day 8, day 9, day 10, day 11, day 12, day 13, or day 14 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, or day 21 of each 3-week cycle, i.e., an anti-CD40 antibody such as SEA-CD40 is administered every three weeks starting on day 1, day 2, day 3, day 4, day 5, day 6, day 7, day 8, day 9, day 10, day 11, day 12, day 13, day 14, day 15, day 16, day 17, day 18, day 19, day 20, or day 21 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, or day 28 of each 4-week cycle, i.e., an anti-CD40 antibody such as SEA-CD40 is administered every four weeks starting on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, or day 28 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, or day 35 of each 5-week cycle, i.e., an anti-CD40 antibody such as SEA-CD40 is administered every five weeks starting on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, or day 35 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, or day 42 of each 6-week cycle, i.e., an anti-CD40 antibody such as SEA-CD40 is administered every six weeks starting on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, or day 42 of a treatment regimen.

In some embodiments, SEA-CD40 is administered on the following two days of each 42-day cycle: day 1 and day 21, day 2 and day 21, day 3 and day 21, day 4 and day 21, day 5 and day 21, day 6 and day 21, day 7 and day 21, day 8 and day 21, day 9 and day 21, day 10 and day 21, day 1 and day 22, day 2 and day 22, day 3 and day 22, day 4 and day 22, day 5 and day 22, day 6 and day 22, day 7 and day 22, day 8 and day 22, day 9 and day 22, day 10 and day 22, day 1 and day 23, day 2 and day 23, day 3 and day 23, day 4 and day 23, day 5 and day 23, day 6 and day 23, day 7 and day 23, day 8 and day 23, day 9 and day 23, day 10 and day 23, day 1 and day 24, day 2 and day 24, day 3 and day 24, day 4 and day 24, day 5 and day 24, day 6 and day 24, day 7 and day 24, day 8 and day 24, day 9 and day 24, day 10 and day 24, day 1 and day 25, day 2 and day 25, day 3 and day 25, day 4 and day 25, day 5 and day 25, day 6 and day 25, day 7 and day 25, day 8 and day 25, day 9 and day 25, day 10 and day 25, day 1 and day 26, day 2 and day 26, day 3 and day 26, day 4 and day 26, day 5 and day 26, day 6 and day 26, day 7 and day 26, day 8 and day 26, day 9 and day 26, day 10 and day 26, day 1 and day 27, day 2 and day 27, day 3 and day 27, day 4 and day 27, day 5 and day 27, day 6 and day 27, day 7 and day 27, day 8 and day 27, day 9 and day 27, day 10 and day 27, day 1 and day 28, day 2 and day 28, day 3 and day 28, day 4 and day 28, day 5 and day 28, day 6 and day 28, day 7 and day 28, day 8 and day 28, day 9 and day 28, day 10 and day 28, day 1 and day 29, day 2 and day 29, day 3 and day 29, day 4 and day 29, day 5 and day 29, day 6 and day 29, day 7 and day 29, day 8 and day 29, day 9 and day 29, day 10 and day 29, day 1 and day 30, day 2 and day 30, day 3 and day 30, day 4 and day 30, day 5 and day 30, day 6 and day 30, day 7 and day 30, day 8 and day 30, day 9 and day 30, or day 10 and day 30.

In this disclosure, administration cycle is described in terms of days or weeks interchangeably as a skilled person would understand. For example, an 1-week administration cycle is the same as a 7-day administration cycle; a 2-week administration cycle is the same as a 14-day administration cycle; a 3-week administration cycle is the same as a 21-week administration cycle; etc.

Dosage and Administration of Pembrolizumab for Treating Cancer in Combination with the Anti-CD40 Antibody

In some embodiments, pembrolizumab is administered at 200 mg once every three weeks. In some embodiments, pembrolizumab is administered at 400 mg once every six weeks. In some embodiments, pembrolizumab is administered at 2 mg/kg (up to 200 mg) every 3 weeks. In some embodiments, pembrolizumab is administered at 2 mg/kg (up to 200 mg) every 3 weeks for pediatrics.

Pembrolizumab can be administered at different intervals including three week intervals and six week intervals. In other words, pembrolizumab can be administered every three weeks or every six weeks. In some embodiments, intervals are based on cycles wherein each cycle can comprise one or more administrations of pembrolizumab. Exemplary lengths of each cycle include three weeks and six weeks. The lengths of cycles can be different from one cycle to the next. Pembrolizumab can be administered on any one or more days in each cycle.

In some embodiments, pembrolizumab is not administered in the first cycle and is administered in the second and the following cycles. In some embodiments, pembrolizumab is not administered in the first cycle of 21 days, and is administered every cycle starting from the second cycle of 21 days. In some embodiments, pembrolizumab is not administered in the first cycle of 42 days, and is administered every cycle starting from the second cycle of 42 days.

In some embodiments, a patient receives a first administration of SEA-CD40 before the patient receives a first administration of pembrolizumab.

In some embodiments, as an alternative to pembrolizumab, another anti-PD-1 antibody or an anti-PD-L1 antibody is used. In some embodiments, the anti-PD-1 antibody is selected from the group consisting of Nivolumab, Cemiplimab-rwlc, Spartalizumab, AK105, Tislelizumab, Dostarlimab, MED10680, AMP-224, and SHR-1210. In some embodiments, the anti-PD-1 antibody is Pembrolizumab, Nivolumab, or Cemiplimab-rwlc. In some embodiments, the anti-PDL1 antibody is selected from the group consisting of Atezolizumab, Durvalumab, Avelumab, SHR-1316, MED14736, BMS-936559/MDX-1105, MSB0010718C, MPDL3280A, or Envafolimab. In some embodiments, the anti-PDL1 antibody is Atezolizumab, Durvalumab, or Avelumab.

### Anti-CD40 Antibody and Pembrolizumab Combination Therapy for Treating Cancer

An anti-CD40 antibody such as SEA-CD40 can be used in combination with pembrolizumab for treating cancer.

A treating regime comprising administering an anti-CD40 antibody such as SEA-CD40 and administering pembrolizumab can have different dosing schedules. In some embodiments, an anti-CD40 antibody such as SEA-CD40 is administered on a two-week cycle and pembrolizumab is administered on a three-week cycle. In some embodiments, an anti-CD40 antibody such as SEA-CD40 is administered on a three-week cycle and pembrolizumab is also administered on a three-week cycle. In some embodiments, an anti-CD40 antibody such as SEA-CD40 is administered on a four-week cycle and pembrolizumab is administered on a three-week cycle. In some embodiments, an anti-CD40 antibody such as SEA-CD40 is administered on a two-week cycle and pembrolizumab is administered on a six-week cycle. In some embodiments, an anti-CD40 antibody such as SEA-CD40 is administered on a three-week cycle and pembrolizumab is administered on a six-week cycle. In some embodiments, an anti-CD40 antibody such as SEA-CD40 is administered on a four-week cycle and pembrolizumab is administered on a six-week cycle. In a preferred embodiment, an anti-CD40 antibody such as SEA-CD40 is administered on a four-week cycle and pembrolizumab is administered on a three-week cycle. In another preferred embodiment, an anti-CD40 antibody such as SEA-CD40 is administered on a four-week cycle and pembrolizumab is administered on a six-week cycle.

In some embodiments, an anti-CD40 antibody such as SEA-CD40 is administered before the administration of pembrolizumab. Giving pembrolizumab after SEA-CD40 may be beneficial, as this timing mitigates the potential for pembrolizumab to bind to immune cells with subsequent immune depletion arising from enhanced clearance of pembrolizumab-bound cells by SEA-CD40. In a combination therapy, the first day of each drug's first cycle of administration all start on the same day. In some embodiments

In some embodiments, an anti-CD40 antibody such as SEA-CD40 is administered on day 1 of the first cycle of the anti-CD40 antibody, and pembrolizumab is administered on day 2 of the first cycle of pembrolizumab. In some embodiments, an anti-CD40 antibody such as SEA-CD40 is administered on day 1 of the first cycle of the anti-CD40 antibody, and pembrolizumab is administered on day 3 of the first cycle of pembrolizumab. In some embodiments, an anti-CD40 antibody such as SEA-CD40 is administered on day 1 of the first cycle of the anti-CD40 antibody, and pembrolizumab is administered on day 4 of the first cycle of pembrolizumab. In some embodiments, an anti-CD40 antibody such as SEA-CD40 is administered on day 1 of the first cycle of the anti-CD40 antibody, and pembrolizumab is administered on day 5 of the first cycle of pembrolizumab. In some embodiments, an anti-CD40 antibody such as SEA-CD40 is administered on day 1 of the first cycle of the anti-CD40 antibody, and pembrolizumab is administered on day 6 of the first cycle of pembrolizumab. In some embodiments, an anti-CD40 antibody such as SEA-CD40 is administered on day 1 of the first cycle of the anti-CD40 antibody, and pembrolizumab is administered on day 7 of the first cycle of pembrolizumab. In some embodiments, an anti-CD40 antibody such as SEA-CD40 is administered on day 1 of the first cycle of the anti-CD40 antibody, and pembrolizumab is administered on day 8 of the first cycle of pembrolizumab. In some embodiments, an anti-CD40 antibody such as SEA-CD40 is administered on day 1 of the first cycle of the anti-CD40 antibody, and pembrolizumab is administered on day 9 of the first cycle of pembrolizumab. In some embodiments, an anti-CD40 antibody such as SEA-CD40 is administered on day 1 of the first cycle of the anti-CD40 antibody, and pembrolizumab is administered on day 10 of the first cycle of pembrolizumab. In some embodiments, an anti-CD40 antibody such as SEA-CD40 is administered on day 1 of the first cycle of the anti-CD40 antibody, and pembrolizumab is administered on day 11 of the first cycle of pembrolizumab. In some embodiments, an anti-CD40 antibody such as SEA-CD40 is administered on day 1 of the first cycle of the anti-CD40 antibody, and pembrolizumab is administered on day 12 of the first cycle of pembrolizumab. In some embodiments, an anti-CD40 antibody such as SEA-CD40 is administered on day 1 of the first cycle of the anti-CD40 antibody, and pembrolizumab is administered on day 13 of the first cycle of pembrolizumab. In some embodiments, an anti-CD40 antibody such as SEA-CD40 is administered on day 1 of the first cycle of the anti-CD40 antibody, and pembrolizumab is administered on day 14 of the first cycle of pembrolizumab.

In some embodiments, an anti-CD40 antibody such as SEA-CD40 is administered on day 2 of the first cycle of the anti-CD40 antibody, and pembrolizumab is administered on day 3 of the first cycle of pembrolizumab. In some embodiments, an anti-CD40 antibody such as SEA-CD40 is administered on day 2 of the first cycle of the anti-CD40 antibody, and pembrolizumab is administered on day 4 of the first cycle of pembrolizumab. In some embodiments, an anti-CD40 antibody such as SEA-CD40 is administered on day 2 of the first cycle of the anti-CD40 antibody, and pembrolizumab is administered on day 5 of the first cycle of pembrolizumab. In some embodiments, an anti-CD40 antibody such as SEA-CD40 is administered on day 2 of the first cycle of the anti-CD40 antibody, and pembrolizumab is administered on day 6 of the first cycle of pembrolizumab. In some embodiments, an anti-CD40 antibody such as SEA-CD40 is administered on day 2 of the first cycle of the anti-CD40 antibody, and pembrolizumab is administered on day 7 of the first cycle of pembrolizumab. In some embodiments, an anti-CD40 antibody such as SEA-CD40 is administered on day 2 of the first cycle of the anti-CD40 antibody, and pembrolizumab is administered on day 8 of the first cycle of pembrolizumab. In some embodiments, an anti-CD40 antibody such as SEA-CD40 is administered on day 2 of the first cycle of the anti-CD40 antibody, and pembrolizumab is administered on day 9 of the first cycle of pembrolizumab. In some embodiments, an anti-CD40 antibody such as SEA-CD40 is administered on day 2 of the first cycle of the anti-CD40 antibody, and pembrolizumab is administered on day 10 of the first cycle of pembrolizumab. In some embodiments, an anti-CD40 antibody such as SEA-CD40 is administered on day 2 of the first cycle of the anti-CD40 antibody, and pembrolizumab is administered on day 11 of the first cycle of pembrolizumab. In some embodiments, an anti-CD40 antibody such as SEA-CD40 is administered on day 2 of the first cycle of the anti-CD40 antibody, and pembrolizumab is administered on day 12 of the first cycle of pembrolizumab. In some embodiments, an anti-CD40 antibody such as SEA-CD40 is administered on day 2 of the first cycle of the anti-CD40 antibody, and pembrolizumab is administered on day 13 of the first cycle of pembrolizumab. In some embodiments, an anti-CD40 antibody such as SEA-CD40 is administered on day 2 of the first cycle of the anti-CD40 antibody, and pembrolizumab is administered on day 14 of the first cycle of pembrolizumab.

In some embodiments, an anti-CD40 antibody such as SEA-CD40 is administered on day 3 of the first cycle of the anti-CD40 antibody, and pembrolizumab is administered on day 4 of the first cycle of pembrolizumab. In some embodiments, an anti-CD40 antibody such as SEA-CD40 is administered on day 3 of the first cycle of the anti-CD40 antibody, and pembrolizumab is administered on day 5 of the first cycle of pembrolizumab. In some embodiments, an anti-CD40 antibody such as SEA-CD40 is administered on day 3 of the first cycle of the anti-CD40 antibody, and pembrolizumab is administered on day 6 of the first cycle of pembrolizumab. In some embodiments, an anti-CD40 antibody such as SEA-CD40 is administered on day 3 of the first cycle of the anti-CD40 antibody, and pembrolizumab is administered on day 7 of the first cycle of pembrolizumab. In some embodiments, an anti-CD40 antibody such as SEA-CD40 is administered on day 3 of the first cycle of the anti-CD40 antibody, and pembrolizumab is administered on day 8 of the first cycle of pembrolizumab. In some embodiments, an anti-CD40 antibody such as SEA-CD40 is administered on day 3 of the first cycle of the anti-CD40 antibody, and pembrolizumab is administered on day 9 of the first cycle of pembrolizumab. In some embodiments, an anti-CD40 antibody such as SEA-CD40 is administered on day 3 of the first cycle of the anti-CD40 antibody, and pembrolizumab is administered on day 10 of the first cycle of pembrolizumab. In some embodiments, an anti-CD40 antibody such as SEA-CD40 is administered on day 3 of the first cycle of the anti-CD40 antibody, and pembrolizumab is administered on day 11 of the first cycle of pembrolizumab. In some embodiments, an anti-CD40 antibody such as SEA-CD40 is administered on day 3 of the first cycle of the anti-CD40 antibody, and pembrolizumab is administered on day 12 of the first cycle of pembrolizumab. In some embodiments, an anti-CD40 antibody such as SEA-CD40 is administered on day 3 of the first cycle of the anti-CD40 antibody, and pembrolizumab is administered on day 13 of the first cycle of pembrolizumab. In some embodiments, an anti-CD40 antibody such as SEA-CD40 is administered on day 3 of the first cycle of the anti-CD40 antibody, and pembrolizumab is administered on day 14 of the first cycle of pembrolizumab.

In some embodiments, an anti-CD40 antibody such as SEA-CD40 is administered on day 4 of the first cycle of the anti-CD40 antibody, and pembrolizumab is administered on day 5 of the first cycle of pembrolizumab. In some embodiments, an anti-CD40 antibody such as SEA-CD40 is administered on day 4 of the first cycle of the anti-CD40 antibody, and pembrolizumab is administered on day 6 of the first cycle of pembrolizumab. In some embodiments, an anti-CD40 antibody such as SEA-CD40 is administered on day 4 of the first cycle of the anti-CD40 antibody, and pembrolizumab is administered on day 7 of the first cycle of pembrolizumab. In some embodiments, an anti-CD40 antibody such as SEA-CD40 is administered on day 4 of the first cycle of the anti-CD40 antibody, and pembrolizumab is administered on day 8 of the first cycle of pembrolizumab. In some embodiments, an anti-CD40 antibody such as SEA-CD40 is administered on day 4 of the first cycle of the anti-CD40 antibody, and pembrolizumab is administered on day 9 of the first cycle of pembrolizumab. In some embodiments, an anti-CD40 antibody such as SEA-CD40 is administered on day 4 of the first cycle of the anti-CD40 antibody, and pembrolizumab is administered on day 10 of the first cycle of pembrolizumab. In some embodiments, an anti-CD40 antibody such as SEA-CD40 is administered on day 4 of the first cycle of the anti-CD40 antibody, and pembrolizumab is administered on day 11 of the first cycle of pembrolizumab. In some embodiments, an anti-CD40 antibody such as SEA-CD40 is administered on day 4 of the first cycle of the anti-CD40 antibody, and pembrolizumab is administered on day 12 of the first cycle of pembrolizumab. In some embodiments, an anti-CD40 antibody such as SEA-CD40 is administered on day 4 of the first cycle of the anti-CD40 antibody, and pembrolizumab is administered on day 13 of the first cycle of pembrolizumab. In some embodiments, an anti-CD40 antibody such as SEA-CD40 is administered on day 4 of the first cycle of the anti-CD40 antibody, and pembrolizumab is administered on day 14 of the first cycle of pembrolizumab.

In some embodiments, an anti-CD40 antibody such as SEA-CD40 is administered on day 5 of the first cycle of the anti-CD40 antibody, and pembrolizumab is administered on day 6 of the first cycle of pembrolizumab. In some embodiments, an anti-CD40 antibody such as SEA-CD40 is administered on day 5 of the first cycle of the anti-CD40 antibody, and pembrolizumab is administered on day 7 of the first cycle of pembrolizumab. In some embodiments, an anti-CD40 antibody such as SEA-CD40 is administered on day 5 of the first cycle of the anti-CD40 antibody, and pembrolizumab is administered on day 8 of the first cycle of pembrolizumab. In some embodiments, an anti-CD40 antibody such as SEA-CD40 is administered on day 5 of the first cycle of the anti-CD40 antibody, and pembrolizumab is administered on day 9 of the first cycle of pembrolizumab. In some embodiments, an anti-CD40 antibody such as SEA-CD40 is administered on day 5 of the first cycle of the anti-CD40 antibody, and pembrolizumab is administered on day 10 of the first cycle of pembrolizumab. In some embodiments, an anti-CD40 antibody such as SEA-CD40 is administered on day 5 of the first cycle of the anti-CD40 antibody, and pembrolizumab is administered on day 11 of the first cycle of pembrolizumab. In some embodiments, an anti-CD40 antibody such as SEA-CD40 is administered on day 5 of the first cycle of the anti-CD40 antibody, and pembrolizumab is administered on day 12 of the first cycle of pembrolizumab. In some embodiments, an anti-CD40 antibody such as SEA-CD40 is administered on day 5 of the first cycle of the anti-CD40 antibody, and pembrolizumab is administered on day 13 of the first cycle of pembrolizumab. In some embodiments, an anti-CD40 antibody such as SEA-CD40 is administered on day 5 of the first cycle of the anti-CD40 antibody, and pembrolizumab is administered on day 14 of the first cycle of pembrolizumab.

In some embodiments, an anti-CD40 antibody such as SEA-CD40 is administered on day 6 of the first cycle of the anti-CD40 antibody, and pembrolizumab is administered on day 7 of the first cycle of pembrolizumab. In some embodiments, an anti-CD40 antibody such as SEA-CD40 is administered on day 6 of the first cycle of the anti-CD40 antibody, and pembrolizumab is administered on day 8 of the first cycle of pembrolizumab. In some embodiments, an anti-CD40 antibody such as SEA-CD40 is administered on day 6 of the first cycle of the anti-CD40 antibody, and pembrolizumab is administered on day 9 of the first cycle of pembrolizumab. In some embodiments, an anti-CD40 antibody such as SEA-CD40 is administered on day 6 of the first cycle of the anti-CD40 antibody, and pembrolizumab is administered on day 10 of the first cycle of pembrolizumab. In some embodiments, an anti-CD40 antibody such as SEA-CD40 is administered on day 6 of the first cycle of the anti-CD40 antibody, and pembrolizumab is administered on day 11 of the first cycle of pembrolizumab. In some embodiments, an anti-CD40 antibody such as SEA-CD40 is administered on day 6 of the first cycle of the anti-CD40 antibody, and pembrolizumab is administered on day 12 of the first cycle of pembrolizumab. In some embodiments, an anti-CD40 antibody such as SEA-CD40 is administered on day 6 of the first cycle of the anti-CD40 antibody, and pembrolizumab is administered on day 13 of the first cycle of pembrolizumab. In some embodiments, an anti-CD40 antibody such as SEA-CD40 is administered on day 6 of the first cycle of the anti-CD40 antibody, and pembrolizumab is administered on day 14 of the first cycle of pembrolizumab.

In some embodiments, an anti-CD40 antibody such as SEA-CD40 is administered on day 7 of the first cycle of the anti-CD40 antibody, and pembrolizumab is administered on day 8 of the first cycle of pembrolizumab. In some embodiments, an anti-CD40 antibody such as SEA-CD40 is administered on day 7 of the first cycle of the anti-CD40 antibody, and pembrolizumab is administered on day 9 of the first cycle of pembrolizumab. In some embodiments, an anti-CD40 antibody such as SEA-CD40 is administered on day 7 of the first cycle of the anti-CD40 antibody, and pembrolizumab is administered on day 10 of the first cycle of pembrolizumab. In some embodiments, an anti-CD40 antibody such as SEA-CD40 is administered on day 7 of the first cycle of the anti-CD40 antibody, and pembrolizumab is administered on day 11 of the first cycle of pembrolizumab. In some embodiments, an anti-CD40 antibody such as SEA-CD40 is administered on day 7 of the first cycle of the anti-CD40 antibody, and pembrolizumab is administered on day 12 of the first cycle of pembrolizumab. In some embodiments, an anti-CD40 antibody such as SEA-CD40 is administered on day 7 of the first cycle of the anti-CD40 antibody, and pembrolizumab is administered on day 13 of the first cycle of pembrolizumab. In some embodiments, an anti-CD40 antibody such as SEA-CD40 is administered on day 7 of the first cycle of the anti-CD40 antibody, and pembrolizumab is administered on day 14 of the first cycle of pembrolizumab.

In some embodiments, an anti-CD40 antibody such as SEA-CD40 is administered on day 8 of the first cycle of the anti-CD40 antibody, and pembrolizumab is administered on day 9 of the first cycle of pembrolizumab. In some embodiments, an anti-CD40 antibody such as SEA-CD40 is administered on day 8 of the first cycle of the anti-CD40 antibody, and pembrolizumab is administered on day 10 of the first cycle of pembrolizumab. In some embodiments, an anti-CD40 antibody such as SEA-CD40 is administered on day 8 of the first cycle of the anti-CD40 antibody, and pembrolizumab is administered on day 11 of the first cycle of pembrolizumab. In some embodiments, an anti-CD40 antibody such as SEA-CD40 is administered on day 8 of the first cycle of the anti-CD40 antibody, and pembrolizumab is administered on day 12 of the first cycle of pembrolizumab. In some embodiments, an anti-CD40 antibody such as SEA-CD40 is administered on day 8 of the first cycle of the anti-CD40 antibody, and pembrolizumab is administered on day 13 of the first cycle of pembrolizumab. In some embodiments, an anti-CD40 antibody such as SEA-CD40 is administered on day 8 of the first cycle of the anti-CD40 antibody, and pembrolizumab is administered on day 14 of the first cycle of pembrolizumab.

In some embodiments, an anti-CD40 antibody such as SEA-CD40 is administered on day 9 of the first cycle of the anti-CD40 antibody, and pembrolizumab is administered on day 10 of the first cycle of pembrolizumab. In some embodiments, an anti-CD40 antibody such as SEA-CD40 is administered on day 9 of the first cycle of the anti-CD40 antibody, and pembrolizumab is administered on day 11 of the first cycle of pembrolizumab. In some embodiments, an anti-CD40 antibody such as SEA-CD40 is administered on day 9 of the first cycle of the anti-CD40 antibody, and pembrolizumab is administered on day 12 of the first cycle of pembrolizumab. In some embodiments, an anti-CD40 antibody such as SEA-CD40 is administered on day 9 of the first cycle of the anti-CD40 antibody, and pembrolizumab is administered on day 13 of the first cycle of pembrolizumab. In some embodiments, an anti-CD40 antibody such as SEA-CD40 is administered on day 9 of the first cycle of the anti-CD40 antibody, and pembrolizumab is administered on day 14 of the first cycle of pembrolizumab.

In some embodiments, an anti-CD40 antibody such as SEA-CD40 is administered on day 10 of the first cycle of the anti-CD40 antibody, and pembrolizumab is administered on day 11 of the first cycle of pembrolizumab. In some embodiments, an anti-CD40 antibody such as SEA-CD40 is administered on day 10 of the first cycle of the anti-CD40 antibody, and pembrolizumab is administered on day 12 of the first cycle of pembrolizumab. In some embodiments, an anti-CD40 antibody such as SEA-CD40 is administered on day 10 of the first cycle of the anti-CD40 antibody, and pembrolizumab is administered on day 13 of the first cycle of pembrolizumab. In some embodiments, an anti-CD40 antibody such as SEA-CD40 is administered on day 10 of the first cycle of the anti-CD40 antibody, and pembrolizumab is administered on day 14 of the first cycle of pembrolizumab.

In some embodiments, an anti-CD40 antibody such as SEA-CD40 is administered on day 11 of the first cycle of the anti-CD40 antibody, and pembrolizumab is administered on day 12 of the first cycle of pembrolizumab. In some embodiments, an anti-CD40 antibody such as SEA-CD40 is administered on day 11 of the first cycle of the anti-CD40 antibody, and pembrolizumab is administered on day 13 of the first cycle of pembrolizumab. In some embodiments, an anti-CD40 antibody such as SEA-CD40 is administered on day 11 of the first cycle of the anti-CD40 antibody, and pembrolizumab is administered on day 14 of the first cycle of pembrolizumab.

In some embodiments, an anti-CD40 antibody such as SEA-CD40 is administered on day 12 of the first cycle of the anti-CD40 antibody, and pembrolizumab is administered on day 13 of the first cycle of pembrolizumab. In some embodiments, an anti-CD40 antibody such as SEA-CD40 is administered on day 12 of the first cycle of the anti-CD40 antibody, and pembrolizumab is administered on day 14 of the first cycle of pembrolizumab.

In some embodiments, an anti-CD40 antibody such as SEA-CD40 is administered on day 13 of the first cycle of the anti-CD40 antibody, and pembrolizumab is administered on day 14 of the first cycle of pembrolizumab.

In some embodiments, the first administration of an anti-CD40 antibody such as SEA-CD40 in the first cycle is 1 day, 2 days, 3 days, 4 days, 5 days, 6 days, 7 days, 8 days, 9 days, 10 days, 11 days, 12 days, 13 days, or 14 days prior to the first administration of pembrolizumab in the first cycle. In some embodiments, the first administration of pembrolizumab is 1 day, days, 3 days, 4 days, 5 days, 6 days, 7 days, 8 days, 9 days, 10 days, 11 days, 12 days, 13 days, or 14 days after the first administration of an anti-CD40 antibody such as SEA-CD40 in the first cycle.

In some embodiments, each dose of the anti-PD-1 antibody is administered at least 1, 2, 3, 4, 5, 6, 7, 8, or 9 days after a dose of the anti-CD40 antibody. In some embodiments, the anti-PD-1 antibody and the anti-CD40 antibody are not administered on the same day. In some embodiments, the interval between the administration of the anti-PD-1 antibody and the administration of the anti-CD40 antibody is at least or about 1, 2, 3, 4, 5, 6, 7, 8, 9, 10, 12, or 24 hours, or at least or about 1, 2, 3, 4, 5, 6, 7, 8, 9, 10, 11, 12, 13, or 14 days.

In some embodiments, pembrolizumab is administered in a cycle of about every 2-4 weeks (e.g., about every 2 weeks, about every 3 weeks, or about every 4 weeks). In some embodiments, pembrolizumab is administered in a cycle of every 14 days, every 15 days, every 16 days, every 17 days, every 18 days, every 19 days, every 20 days, every 21 days, every 22 days, every 23 days, every 24 days, every 25 days, every 26 days, every 27 days, or every 28 days. In some embodiments, pembrolizumab is administered in a cycle of about every 5-7 weeks (e.g., about every 5 weeks, about every 6 weeks, or about every 7 weeks). In some embodiments, pembrolizumab is administered in a cycle of every 35 days, every 36 days, every 37 days, every 38 days, every 39 days, every 40 days, every 41 days, every 42 days, every 43 days, every 44 days, every 45 days, every 46 days, every 47 days, every 48 days, or every 49 days.

In some embodiments, pembrolizumab is administered every 3 weeks at a dose of about 200 mg. In some embodiments, pembrolizumab is administered every 3 weeks at a dose of 200 mg. In some embodiments, pembrolizumab is administered every 3 at a dose of about 2 mg/kg. In some embodiments, pembrolizumab is administered every 6 weeks at a dose of 400 mg.

Combination Therapy with Anti-CD40 Antibody and Chemotherapy

The combination therapy of an anti-CD40 antibody such as SEA-CD40 can be combined with chemotherapy. In some embodiments, the combination therapy of an anti-CD40 antibody such as SEA-CD40 and pembrolizumab can be further combined with chemotherapy.

In most humans, millions of cells die via apoptosis and are removed without generating an immune response. However, after treatment with some chemotherapeutic agents, immune cells have been observed to infiltrate tumors. Thus, some tumor cells killed by chemotherapeutic agents act as vaccines and raise a tumor-specific immune response. This phenomenon is referred to as immunogenic cell death (ICD). See, e.g., Kroemer et al., Annu. Rev. Immunol., 31:51-72 (2013). The ability of a chemotherapeutic agent to induce ICD can be determined experimentally. Two criteria must be met. First, injection of an immunocompetent mouse with cancer cells that have been treated in vitro with a chemotherapeutic agent must elicit a protective immune response that is specific for tumor antigens, in the absence of adjuvant. Second, ICD occurring in vivo, e.g., a mouse syngeneic model with treatment using a potential ICD-inducing chemotherapeutic agent, must drive an immune response in the tumor that is dependent on the immune system.

Chemotherapeutic agents that induce ICD include, e.g., anthracyclines, anti-EGFR antibodies, bortezomib, cyclophosphamide, gemcitabine, irradiation of the tumor, and oxaliplatin. Chemotherapeutic agents also include gemcitabine, dacarbazine, temozolomide, paclitaxel, albumin-bound paclitaxel, carboplatin, and pemetrexed. A combination of an anti-CD40 antibody such as SEA-CD40 and pembrolizumab can be used in combination with any of these chemotherapy agents to generate an enhanced immune response and treat cancer in a patient. In some embodiments, the combination of an anti-CD40 antibody such as SEA-CD40 and pembrolizumab is used in combination with one or more of gemcitabine, dacarbazine, temozolomide, paclitaxel, albumin-bound paclitaxel, carboplatin, or pemetrexed. ABRAXANE® (nab-paclitaxel) is a brand name of paclitaxel containing albumin-bound paclitaxel. In some embodiments, the combination of an anti-CD40 antibody such as SEA-CD40 and pembrolizumab is used in combination with both the chemotherapeutic agent gemcitabine and paclitaxel/nab-paclitaxel.

In some embodiments, the combination therapy includes an anti-CD40 antibody such as SEA-CD40, pembrolizumab, and chemotherapy. In some embodiments, chemotherapy used in the combination includes gemcitabine or paclitaxel. In some embodiments, chemotherapy used in the combination includes both gemcitabine and paclitaxel. In some embodiments, paclitaxel is nab-paclitaxel, e.g., ABRAXANE®.

Chemotherapy used in the combination can be administered in cycles. In some embodiments, the cycle is 1 week, i.e., chemotherapy is administered every week. In some embodiments, the cycle is 2 weeks, i.e., chemotherapy is administered every 2 weeks. In some embodiments, the cycle is 3 weeks, i.e., chemotherapy is administered every 3 weeks. In some embodiments, the cycle is 4 weeks, i.e., chemotherapy is administered every 4 weeks. In some embodiments, the cycle is 5 weeks, i.e., chemotherapy is administered every 5 weeks. In some embodiments, the cycle is 6 weeks, i.e., chemotherapy is administered every 6 weeks. In some embodiments, the cycle is 7 weeks, i.e., chemotherapy is administered every 7 weeks. In some embodiments, the cycle is 8 weeks, i.e., chemotherapy is administered every 8 weeks. In each cycle, chemotherapy can be administered one or more times.

In some embodiments, chemotherapy used in the combination is administered in a 4 week cycle, i.e., chemotherapy is administered every 4 weeks, wherein chemotherapy is administered three times in each cycle. In some embodiments, chemotherapy administered in the combination is administered on days 1, 8, and 15 in each cycle.

In some embodiments, chemotherapy used in the combination is administered in a cycle of about every 3-5 weeks (e.g., about every 3 weeks, about every 4 weeks, or about every 5 weeks). In some embodiments, chemotherapy used in the combination is administered in a cycle of every 21 days, every 22 days, every 23 days, every 24 days, every 25 days, every 26 days, every 27 days, every 28 days, every 29 days, every 30 days, every 31 days, every 32 days, every 33 days, every 34 days, or every 35 days.

In some embodiments, chemotherapy used in the combination includes gemcitabine (e.g., INFUGEM™) and/or paclitaxel (e.g., ABRAXANE©). In some embodiments, gemcitabine is administered 1 time, 2 times, 3 times, 4 times, or 5 times in each cycle. In some embodiments, gemcitabine is administered on days 1, 8, and 15 in each cycle (e.g., a 28-day cycle). In some embodiments, gemcitabine is administered on days 1 and 8 of each cycle (e.g., a 21-day cycle). In some embodiments, gemcitabine is administered at a dose of about 800-1500 mg/m² (e.g., about 800 mg/m², about 850 mg/m², about 900 mg/m², about 950 mg/m², about 1000 mg/m², about 1050 mg/m², about 1100 mg/m², about 1150 mg/m², about 1200 mg/m², about 1250 mg/m², about 1300 mg/m², about 1350 mg/m², about 1400 mg/m², about 1450 mg/m², or about 1500 mg/m²), e.g., over about 20-60 minutes (e.g., about 20 minutes, about 30 minutes, about 40 minutes, about 50 minutes, or about 60 minutes). In some embodiments, paclitaxel is administered 1 time, 2 times, 3 times, 4 times, or 5 times in each cycle. In some embodiments, paclitaxel is administered on days 1, 8, and 15 of each cycle (e.g., a 21-day cycle, or a 28-day cycle). In some embodiments, paclitaxel is administered at a dose of about 50-300 mg/m² (e.g., about 50 mg/m², about 60 mg/m², about 70 mg/m², about 80 mg/m², about 90 mg/m², about 100 mg/m², about 110 mg/m², about 120 mg/m², about 125 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg//m², about 250 mg/m², about 260 mg/m², about 270 mg/m², about 280 mg/m², about 290 mg/m², or about 300 mg/m²), e.g., over about 20-60 minutes (e.g., about 20 minutes, about 30 minutes, about 40 minutes, about 50 minutes, or about 60 minutes).

In a preferred embodiment, the first administration of the first cycle of chemotherapy is given prior to the administration of an anti-CD40 antibody such as SEA-CD40 to allow for antigen release to occur from tumor cells as a consequence of chemotherapy. In some embodiments, the chemotherapy is given 1 day prior to the administration of an anti-CD40 antibody such as SEA-CD40. In some embodiments, the chemotherapy is given 2 days prior to the administration of an anti-CD40 antibody such as SEA-CD40. In some embodiments, the chemotherapy is given 3 days prior to the administration of an anti-CD40 antibody such as SEA-CD40. This timing is anticipated to enhance the potential for an anti-CD40 antibody such as SEA-CD40 to lead to an anti-tumor immune response. Specifically, an anti-CD40 antibody such as SEA-CD40 can stimulate antigen update and presentation—and thus is expected to be most effective in the setting of increased levels of circulating antigen. Additionally, waiting 1-3 days after chemotherapy before administering an anti-CD40 antibody such as SEA-CD40 may mitigate the potential for synergistic toxicity.

In some embodiments, each dose of the anti-CD40 antibody is administered at least 1, 2, 3, 4, 5, 6, 7, 8, or 9 days after a dose of the chemotherapy. In some embodiments, the chemotherapy and the anti-CD40 antibody are not administered on the same day. In some embodiments, the interval between the administration of the chemotherapy and the administration of the anti-CD40 antibody is at least or about 1, 2, 3, 4, 5, 6, 7, 8, 9, 10, 12, or 24 hours, or at least or about 1, 2, 3, 4, 5, 6, 7, 8, 9, 10, 11, 12, 13, or 14 days.

In some embodiments, the chemotherapy is administered on day 1 of the first cycle of the chemotherapy, an anti-CD40 antibody such as SEA-CD40 is administered on day 3 of the first cycle of the anti-CD40 antibody, and pembrolizumab is administered on day 8 of the first cycle of pembrolizumab, wherein day 1 of the chemotherapy cycle, the anti-CD40 antibody cycle, and the pembrolizumab cycle starts on the same day.

In a preferred embodiment, the combination therapy includes a chemotherapy administered on day 1, day 8, and day 16 of a 28 day cycle, an anti-CD40 antibody such as SEA-CD40 administered on day 3 of a 28 day cycle, and pembrolizumab administered on day 8 of a 42 day cycle. In some embodiments, the combination therapy includes a chemotherapy administered on day 1, day 8, and day 16 of a 28 day cycle, an anti-CD40 antibody such as SEA-CD40 administered on day 3 of a 28 day cycle, and pembrolizumab administered on day 8 of a 21 day cycle. In a preferred embodiment, the combination therapy includes a chemotherapy administered on day 1, day 8, and day 15 of a 28 day cycle, an anti-CD40 antibody such as SEA-CD40 administered on day 3 of a 28 day cycle, and pembrolizumab administered on day 8 of a 42 day cycle. In some embodiments, the combination therapy includes a chemotherapy administered on day 1, day 8, and day 15 of a 28 day cycle, an anti-CD40 antibody such as SEA-CD40 administered on day 3 of a 28 day cycle, and pembrolizumab administered on day 8 of a 21 day cycle. In some embodiments, the chemotherapy includes both gemcitabine and paclitaxel. In some embodiments, paclitaxel is nab-paclitaxel, e.g., ABRAXANE©.

In one aspect, the disclosure relates to treating a pancreatic cancer with a combination of chemotherapy, pembrolizumab, and SEA-CD40, wherein the chemotherapy is administered on days 1, 8, and 15 of each 28-day cycle, wherein SEA-CD40 is administered on day 3 of each 28-day cycle, and wherein pembrolizumab is administered on day 8 of each 42-day cycle. In some embodiments, the chemotherapy consists of gemcitabine and nab-paclitaxel (ABRAXANE©). In some embodiments, SEA-CD40 is administered intravenously. In some embodiments, SEA-CD40 is administered subcutaneously. In some embodiments, pembrolizumab is administered at 400 mg. In some embodiments, pembrolizumab is administered at 200 mg. In some embodiments, SEA-CD40 is administered at 10 μg/kg. In some embodiments, SEA-CD40 is administered at 30 μg/kg. In some embodiments, the pancreatic cancer is pancreatic ductal adenocarcinoma (PDAC). In some embodiments, the pancreatic cancer is metastatic pancreatic ductal adenocarcinoma (PDAC).

In another aspect, this disclosure relates to treating a pancreatic cancer with a combination of chemotherapy, an anti-PD-1 antibody, and SEA-CD40, wherein the chemotherapy is administered on days 1, 8, and 15 of each each 28-day cycle, wherein SEA-CD40 is administered on day 3 of each 28-day cycle, and wherein the anti-PD-1 antibody is administered on day 8 of each 42-day cycle. In some embodiments, the chemotherapy consists of gemcitabine and nab-paclitaxel (ABRAXANE©). In some embodiments, SEA-CD40 is administered intravenously. In some embodiments, SEA-CD40 is administered subcutaneously. In some embodiments, SEA-CD40 is administered at 10 μg/kg. In some embodiments, SEA-CD40 is administered at 30 μg/kg. In some embodiments, the pancreatic cancer is pancreatic ductal adenocarcinoma (PDAC). In some embodiments, the pancreatic cancer is metastatic pancreatic ductal adenocarcinoma (PDAC). In some embodiments, the anti-PD-1 antibody is pembrolizumab. In some embodiments, pembrolizumab is administered at 400 mg. In some embodiments, pembrolizumab is administered at 200 mg.

### Combination for Treating Melanoma

Melanoma is a type of skin cancer that develops when melanocytes (the cells that give the skin its tan or brown color) start to grow out of control. Cancer starts when cells in the body become cancerous.

The combination therapies in this disclosure can be used to treat melanoma. In some embodiments, the melanoma is relapsed melanoma. In some embodiments, the melanoma is refractory melanoma. In some embodiments, the melanoma is uveal melanoma. In some embodiments, the melanoma is metastatic uveal melanoma. In some embodiments, the melanoma is PD-(L)1 naïve melanoma. A combination of a SEA-CD40 and pembrolizumab can be used for treating any of the above melanoma.

In some embodiments, both SEA-CD40 and pembrolizumab are administered in a cycle of 21 days. In some embodiments, the first administration of SEA-CD40 is administered before the first administration of pembrolizumab. In some embodiments, SEA-CD40 and pembrolizumab are both administered once in each 21-day cycle. For each 21-day cycle, SEA-CD40 and pembrolizumab can be administered on the following days respectively: day 1 and day 2, day 1 and day 3, day 1 and day 4, day 1 and day 5, day 1 and day 6, day 1 and day 7, day 1 and day 8, day 1 and day 9, day 1 and day 10, day 2 and day 3, day 2 and day 4, day 2 and day 5, day 2 and day 6, day 2 and day 7, day 2 and day 8, day 2 and day 9, day 2 and day 10, day 2 and day 11, day 3 and day 4, day 3 and day 5, day 3 and day 6, day 3 and day 7, day 3 and day 8, day 3 and day 9, day 3 and day 10, day 3 and day 11, day 3 and day 12, day 4 and day 5, day 4 and day 6, day 4 and day 7, day 4 and day 8, day 4 and day 9, day 4 and day 10, day 4 and day 11, day 4 and day 12, day 4 and day 13, day 5 and day 6, day 5 and day 7, day 5 and day 8, day 5 and day 9, day 5 and day 10, day 5 and day 11, day 5 and day 12, day 5 and day 13, or day 5 and day 14. In some embodiments, SEA-CD40 is administered on day 1, and pembrolizumab is administered on day 8. In some embodiments, SEA-CD40 is administered on day 3, and pembrolizumab is administered on day 8. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 200 mg in each 21-day cycle. In some embodiments, SEA-CD40 is administered at a dose of g/kg and pembrolizumab is administered at 200 mg in each of the 21-day cycle. In some embodiments, the SEA-CD40 is administered at a dose of 30 μg/kg and pembrolizumab is administered at 200 mg in each of the 21-day cycle. In some embodiments, pembrolizumab is not administered in the first 21-day cycle, and is administered for each cycle starting from the second 21-day cycle.

In some embodiments, both SEA-CD40 and pembrolizumab are administered in a cycle of 42 days. In some embodiments, the first administration of SEA-CD40 is administered before the first administration of pembrolizumab. In some embodiments, SEA-40 is administered twice and pembrolizumab is administered once in each 42-day cycle. For each 42-day cycle, the first administrations of SEA-CD40, the second administration of SEA-CD40 and the one administration of pembrolizumab are on the following days respectively: day 1, day 21, and day 5; day 1, day 22, and day 5; day 1, day 23, and day 5; day 2, day 21, and day 5; day 2, day 22, and day 5; day 2, day 23, and day 5; day 3, day 21, and day 5; day 3, day 22, and day 5; day 3, day 23, and day 5; day 1, day 21, and day 6; day 1, day 22, and day 6; day 1, day 23, and day 6; day 2, day 21, and day 6; day 2, day 22, and day 6; day 2, day 23, and day 6; day 3, day 21, and day 6; day 3, day 22, and day 6; day 3, day 23, and day 6; day 1, day 21, and day 7; day 1, day 22, and day 7; day 1, day 23, and day 7; day 2, day 21, and day 7; day 2, day 22, and day 7; day 2, day 23, and day 7; day 3, day 21, and day 7; day 3, day 22, and day 7; day 3, day 23, and day 7; day 1, day 21, and day 8; day 1, day 22, and day 8; day 1, day 23, and day 8; day 2, day 21, and day 8; day 2, day 22, and day 8; day 2, day 23, and day 8; day 3, day 21, and day 8; day 3, day 22, and day 8; day 3, day 23, and day 8; day 1, day 21, and day 9; day 1, day 22, and day 9; day 1, day 23, and day 9; day 2, day 21, and day 9; day 2, day 22, and day 9; day 2, day 23, and day 9; day 3, day 21, and day 9; day 3, day 22, and day 9; day 3, day 23, and day 9; day 1, day 21, and day 10; day 1, day 22, and day 10; day 1, day 23, and day 10; day 2, day 21, and day 10; day 2, day 22, and day 10; day 2, day 23, and day 10; day 3, day 21, and day 10; day 3, day 22, and day 10; or day 3, day 23, and day 10. In some embodiments, SEA-CD40 is administered on day 1 and day 21 of each 42-day cycle, and pembrolizumab is administered on day 8 of each 42-day cycle. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 400 mg in each 42-day cycle. In some embodiments, SEA-CD40 is administered at a dose of 10 μg/kg and pembrolizumab is administered at 400 mg in each of the 42-day cycle. In some embodiments, the SEA-CD40 is administered at a dose of 30 μg/kg and pembrolizumab is administered at 400 mg in each of the 42-day cycle. In some embodiments, pembrolizumab is not administered in the first 42-day cycle, and is administered for each cycle starting from the second 42-day cycle.

### Combination for Treating NSCLC

Lung cancer is a type of cancer that starts in the lungs. Cancer starts when cells in the body become cancerous. About 80% to 85% of lung cancers are NSCLC. The main subtypes of NSCLC are adenocarcinoma, squamous cell carcinoma, and large cell carcinoma. These subtypes, which start from different types of lung cells are grouped together as NSCLC because their treatment and prognoses (outlook) are often similar. Squamous NSCLC starts in squamous cells, which are flat cells that line the inside of the airways in the lungs. Non-squamous NSCLC will generally originate in peripheral lung tissue.

The combination therapies in this disclosure can be used to treat NSCLC. In some embodiments, the NSCLC is squamous NSCLC. In some embodiments, the NSCLC is non-squamous NSCLC.

In some embodiments, this disclosure relates to treating non-squamous NSCLC with a combination of SEA-CD40, pembrolizumab, carboplatin, and pemetrexed. In some embodiments, SEA-CD40, pembrolizumab, carboplatin, and pemetrexed are all administered in 21-day cycles. In some embodiments, carboplatin is administered on day 1 of the first four cycles (cycles 1-4) and not administered in the following cycles. In some embodiments, pemetrexed is administered on day 1 of each 21-day cycle. In some embodiments, SEA-CD40 and pembrolizumab are administered on the following days of each 21-day cycle, respectively: day 1 and day 1, day 1 and day 2, day 1 and day 3, day 1 and day 4, day 1 and day 5, day 1 and day 6, day 1 and day 7, day 1 and day 8, day 1 and day 9, day 1 and day 10, day 2 and day 1, day 2 and day 2, day 2 and day 3, day 2 and day 4, day 2 and day 5, day 2 and day 6, day 2 and day 7, day 2 and day 8, day 2 and day 9, day 2 and day 10, day 3 and day 1, day 3 and day 2, day 3 and day 3, day 3 and day 4, day 3 and day 5, day 3 and day 6, day 3 and day 7, day 3 and day 8, day 3 and day 9, day 3 and day 10, day 4 and day 1, day 4 and day 2, day 4 and day 3, day 4 and day 4, day 4 and day 5, day 4 and day 6, day 4 and day 7, day 4 and day 8, day 4 and day 9, day 4 and day 10, day 5 and day 1, day 5 and day 2, day 5 and day 3, day 5 and day 4, day 5 and day 5, day 5 and day 6, day 5 and day 7, day 5 and day 8, day 5 and day 9, day 5 and day 10, day 6 and day 1, day 6 and day 2, day 6 and day 3, day 6 and day 4, day 6 and day 5, day 6 and day 6, day 6 and day 7, day 6 and day 8, day 6 and day 9, day 6 and day 10, day 7 and day 1, day 7 and day 2, day 7 and day 3, day 7 and day 4, day 7 and day 5, day 7 and day 6, day 7 and day 7, day 7 and day 8, day 7 and day 9, day 7 and day 10, day 8 and day 1, day 8 and day 2, day 8 and day 3, day 8 and day 4, day 8 and day 5, day 8 and day 6, day 8 and day 7, day 8 and day 8, day 8 and day 9, day 8 and day 10, day 9 and day 1, day 9 and day 2, day 9 and day 3, day 9 and day 4, day 9 and day 5, day 9 and day 6, day 9 and day 7, day 9 and day 8, day 9 and day 9, day 9 and day 10, day 10 and day 1, day 10 and day 2, day 10 and day 3, day 10 and day 4, day 10 and day 5, day 10 and day 6, day 10 and day 7, day 10 and day 8, day 10 and day 9, or day 10 and day 10. In some embodiments, SEA-CD40 is administered on day 3 of each 21-day cycle and pembrolizumab is administered on day 1 of each 21-day cycle. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 200 mg in each 21-day cycle. In some embodiments, pemetrexed is administered at a dose of 500 mg per square meter. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, carboplatin is administered at a dose of area under the concentration-time curve (AUC) 5 mg per milliliter per minute. In some embodiments, pembrolizumab is not administered in the first 21-day cycle, and is administered for each cycle starting from the second 21-day cycle. In some embodiments, the first administration of SEA-CD40 is before the first administration of pembrolizumab.

In some embodiments, this disclosure relates to treating non-squamous NSCLC with a combination of SEA-CD40, pembrolizumab, carboplatin, and pemetrexed. In some embodiments, SEA-CD40, pembrolizumab, carboplatin, and pemetrexed are all administered in 21-day cycles. In some embodiments, SEA-CD40, pembrolizumab, carboplatin, and pemetrexed are all administered once in each 21-day cycle. In some embodiments, pemetrexed is administered on day 1 of each 21-day cycle. In some embodiments, carboplatin is administered on day 1 of each 21-day cycle. In some embodiments, carboplatin is only administered in the first four 21-day cycles (Cycles 1-4). In some embodiments, SEA-CD40 is administered on day 1 of each 21-day cycle. In some embodiments, SEA-CD40 is administered on day 3 of each 21-day cycle. In some embodiments, pembrolizumab is administered on day 1 of each 21-day cycle. In some embodiments, the administration of SEA-CD40, the administration of pembrolizumab, the administration of pemetrexed, and the administration of carboplatin are on the following days of each 21-day cycle, respectively, except that carboplatin is administered only in the first four 21-day cycles: day 1, day 1, day 1, and day 1; day 1, day 2, day 1, and day 1; day 1, day 3, day 1, and day 1; day 1, day 4, day 1, and day 1; day 1, day 5, day 1, and day 1; day 1, day 6, day 1, and day 1; day 1, day 7, day 1, and day 1; day 1, day 8, day 1, and day 1; day 2, day 1, day 1, and day 1; day 2, day 2, day 1, and day 1; day 2, day 3, day 1, and day 1; day 2, day 4, day 1, and day 1; day 2, day 5, day 1, and day 1; day 2, day 6, day 1, and day 1; day 2, day 7, day 1, and day 1; day 2, day 8, day 1, and day 1; day 3, day 1, day 1, and day 1; day 3, day 2, day 1, and day 1; day 3, day 3, day 1, and day 1; day 3, day 4, day 1, and day 1; day 3, day 5, day 1, and day 1; day 3, day 6, day 1, and day 1; day 3, day 7, day 1, and day 1; or day 3, day 8, day 1, and day 1. In some embodiments, SEA-CD40 is administered on day 3 of each 21-day cycle; pembrolizumab is administered on day 1 of each 21-day cycle; pemetrexed is administered on day 1 of each 21-day cycle; and carboplatin is administered on day 1 of the first four 21-day cycles. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 200 mg in each 21-day cycle. In some embodiments, pemetrexed is administered at a dose of 500 mg per square meter. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, carboplatin is administered at a dose of area under the concentration-time curve (AUC) 5 mg per milliliter per minute. In some embodiments, pembrolizumab is not administered in the first 21-day cycle, and is administered for each cycle starting from the second 21-day cycle. In some embodiments, the first administration of SEA-CD40 is before the first administration of pembrolizumab.

In some embodiments, SEA-CD40, pemetrexed, and carboplatin are administered in 21-day cycles, and pembrolizumab is administered in 42-day cycles, to treat non-squamous NSCLC. In some embodiments, pemetrexed and carboplatin are both administered in day 1 of each 21-day cycle, SEA-CD40 is administered on any of day 1, day 2, or day 3 of each 21-day cycle, and pembrolizumab is administered on any of day 1, day 2, day 3, day 4, day 5, day 6, day 7, day 8, day 9, or day 10 of each 42-day cycle. In some embodiments, SEA-CD40 is administered on day 3 of each 21-day cycle, pemetrexed and carboplatin are both administered on day 1 of each 21-day cycle, and pembrolizumab is administered on day 1 of each 42-day cycle. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 400 mg in each 42-day cycle. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, pemetrexed is administered at a dose of 500 mg per square meter. In some embodiments, carboplatin is administered at a dose of area under the concentration-time curve (AUC) 5 mg per milliliter per minute. In some embodiments, pembrolizumab is not administered in the first 42-day cycle, and is administered for each cycle starting from the second 42-day cycle. In some embodiments, the first administration of SEA-CD40 is before the first administration of pembrolizumab.

In some embodiments, this disclosure relates to treating squamous NSCLC with a combination of SEA-CD40, pembrolizumab, carboplatin, and paclitaxel or nab-paclitaxel. In some embodiments, SEA-CD40, pembrolizumab, carboplatin, and paclitaxel or nab-paclitaxel are all administered in 21-day cycles. In some embodiments, carboplatin is administered on day 1 of each 21-day cycle. In some embodiments, carboplatin is only administered in the first four 21-day cycles (Cycles 1-4). In some embodiments, SEA-CD40 is administered on day 1 of each 21-day cycle. In some embodiments, SEA-CD40 is administered on day 3 of each 21-day cycle. In some embodiments, pembrolizumab is administered on day 1 of each 21-day cycle. In some embodiments, paclitaxel is administered on day 1 of each 21-day cycle at 200 mg per square meter of body-surface area. In some embodiments, nab-paclitaxel is administered at 100 mg per square meter on days 1, 8, and 15 of each 21-day cycle. In some embodiments, squamous NSCLC is treated with a combination of SEA-CD40, pembrolizumab, carboplatin and paclitaxel, wherein the administration of SEA-CD40, the administration of pembrolizumab, the administration of carboplatin, and the administration of paclitaxel are on the following days of each 21-day cycle respectively, except that carboplatin is administered on only the first four 21-day cycles: day 1, day 1, day 1, and day 1; day 1, day 2, day 1, and day 1; day 1, day 3, day 1, and day 1; day 1, day 4, day 1, and day 1; day 1, day 5, day 1, and day 1; day 1, day 6, day 1, and day 1; day 1, day 7, day 1, and day 1; day 1, day 8, day 1, and day 1; day 2, day 1, day 1, and day 1; day 2, day 2, day 1, and day 1; day 2, day 3, day 1, and day 1; day 2, day 4, day 1, and day 1; day 2, day 5, day 1, and day 1; day 2, day 6, day 1, and day 1; day 2, day 7, day 1, and day 1; day 2, day 8, day 1, and day 1; day 3, day 1, day 1, and day 1; day 3, day 2, day 1, and day 1; day 3, day 3, day 1, and day 1; day 3, day 4, day 1, and day 1; day 3, day 5, day 1, and day 1; day 3, day 6, day 1, and day 1; day 3, day 7, day 1, and day 1; or day 3, day 8, day 1, and day 1. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 200 mg in each 21-day cycle. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, carboplatin is administered at a dose of area under the concentration-time curve (AUC) 6 mg per milliliter per minute. In some embodiments, paclitaxel is administered at 200 mg per square meter of body-surface area for each administration. In some embodiments, pembrolizumab is not administered in the first 21-day cycle, and is administered for each cycle starting from the second 21-day cycle. In some embodiments, the first administration of SEA-CD40 is before the first administration of pembrolizumab.

In some embodiments, squamous NSCLC is treated with a combination of SEA-CD40, pembrolizumab, carboplatin and nab-paclitaxel. In some embodiments, SEA-CD40, pembrolizumab, carboplatin, and nab-paclitaxel are all administered in 21-day cycles. In some embodiments, carboplatin is administered on day 1 of each 21-day cycle. In some embodiments, carboplatin is only administered in the first four 21-day cycles (Cycles 1-4). In some embodiments, SEA-CD40 is administered on day 1 of each 21-day cycle. In some embodiments, SEA-CD40 is administered on day 3 of each 21-day cycle. In some embodiments, pembrolizumab is administered on day 1 of each 21-day cycle. In some embodiments, nab-paclitaxel is administered on days 1, 8, and 15 of each 21-day cycle at 100 mg per square meter of body-surface area. In some embodiments, squamous NSCLC is treated with a combination of SEA-CD40, pembrolizumab, carboplatin and nab-paclitaxel, wherein 1) carboplatin is administered in only the first four 21-day cycles and administered on day 1 of each of the first four 21-day cycles; 2) nab-paclitaxel is administered on days 1, 8, and 15 of each 21-day cycles; and 3) the administration of SEA-CD40 and the administration of pembrolizumab are on the following days of each 21-day cycle respectively: day 1 and day 1, day 1 and day 2, day 1 and day 3, day 1 and day 4, day 1 and day 5, day 1 and day 6, day 1 and day 7, day 1 and day 8, day 2 and day 1, day 2 and day 2, day 2 and day 3, day 2 and day 4, day 2 and day 5, day 2 and day 6, day 2 and day 7, day 2 and day 8, day 3 and day 1, day 3 and day 2, day 3 and day 3, day 3 and day 4, day 3 and day 5, day 3 and day 6, day 3 and day 7, or day 3 and day 8. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 200 mg in each 21-day cycle. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, carboplatin is administered at a dose of area under the concentration-time curve (AUC) 6 mg per milliliter per minute. In some embodiments, nab-paclitaxel is administered at 100 mg per square meter for each administration. In some embodiments, pembrolizumab is not administered in the first 21-day cycle, and is administered for each cycle starting from the second 21-day cycle. In some embodiments, the first administration of SEA-CD40 is before the first administration of pembrolizumab.

In some embodiments, squamous NSCLC is treated with a combination of SEA-CD40, carboplatin and paclitaxel in 21-day cycles, and pembrolizumab in 42-day cycles, wherein 1) carboplatin is administered in only the first four 21-day cycles on day 1 of each of the first four 21-day cycles; 2) paclitaxel is administered on day 1 of each 21-day cycle; and 3) SEA-CD40 and pembrolizumab are administered on the following days of their respective cycles (21-day cycle for SEA-CD40 and 42-day cycle for pembrolizumab): day 1 and day 1, day 1 and day 2, day 1 and day 3, day 1 and day 4, day 1 and day 5, day 1 and day 6, day 1 and day 7, day 1 and day 8, day 2 and day 1, day 2 and day 2, day 2 and day 3, day 2 and day 4, day 2 and day 5, day 2 and day 6, day 2 and day 7, day 2 and day 8, day 3 and day 1, day 3 and day 2, day 3 and day 3, day 3 and day 4, day 3 and day 5, day 3 and day 6, day 3 and day 7, or day 3 and day 8. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 400 mg in each 42-day cycle. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, carboplatin is administered at a dose of area under the concentration-time curve (AUC) 6 mg per milliliter per minute. In some embodiments, paclitaxel is administered at 200 mg per square meter of body-surface area for each administration. In some embodiments, pembrolizumab is not administered in the first 42-day cycle, and is administered for each cycle starting from the second 42-day cycle. In some embodiments, the first administration of SEA-CD40 is before the first administration of pembrolizumab.

In some embodiments, squamous NSCLC is treated with a combination of SEA-CD40, carboplatin and nab-paclitaxel in 21-day cycles, and pembrolizumab in 42-day cycles, wherein 1) carboplatin is administered in only the first four 21-day cycles on day 1 of each of the first four 21-day cycles; 2) nab-paclitaxel is administered on days 1, 8, and 15 of each 21-day cycle; and 3) SEA-CD40 and pembrolizumab are administered on the following days of their respective cycles (21-day cycle for SEA-CD40 and 42-day cycle for pembrolizumab): day 1 and day 1, day 1 and day 2, day 1 and day 3, day 1 and day 4, day 1 and day 5, day 1 and day 6, day 1 and day 7, day 1 and day 8, day 2 and day 1, day 2 and day 2, day 2 and day 3, day 2 and day 4, day 2 and day 5, day 2 and day 6, day 2 and day 7, day 2 and day 8, day 3 and day 1, day 3 and day 2, day 3 and day 3, day 3 and day 4, day 3 and day 5, day 3 and day 6, day 3 and day 7, or day 3 and day 8. In some embodiments, SEA-CD40 is administered at a dose of 10 μg/kg for each administration. In some embodiments, SEA-CD40 is administered at a dose of 30 μg/kg for each administration. In some embodiments, pembrolizumab is administered at 400 mg in each 42-day cycle. In some embodiments, carboplatin is administered at a dose of area under the concentration-time curve (AUC) 2-8 mg per milliliter per minute. In some embodiments, carboplatin is administered at a dose of area under the concentration-time curve (AUC) 2 mg, 3 mg, 4 mg, 5 mg, 6 mg, 7 mg, or 8 mg per milliliter per minute. In some embodiments, carboplatin is administered at a dose of area under the concentration-time curve (AUC) 6 mg per milliliter per minute. In some embodiments, nab-paclitaxel is administered at 100 mg per square meter of body-surface area for each administration. In some embodiments, pembrolizumab is not administered in the first 42-day cycle, and is administered for each cycle starting from the second 42-day cycle. In some embodiments, the first administration of SEA-CD40 is before the first administration of pembrolizumab.

### Expression Levels of PD-L1

The level of expression of PD-L1 in a cancer in a subject can be measured prior to administering any composition or utilizing any method disclosed herein. The level of expression can be determined by any methods known in the art.

In order to assess the level of expression of PD-L1, in some embodiments, a cancer tissue sample can be obtained from the subject who is in need of the therapy. In another embodiment, the assessment of level of expression of PD-L1 can be achieved without obtaining a cancer tissue sample. In some embodiments, selecting a suitable subject includes (i) optionally providing a cancer tissue sample obtained from a subject, the cancer tissue sample comprising cancer cells and/or cancer-infiltrating inflammatory cells; and (ii) assessing the proportion of cells in the cancer tissue sample that express PD-L1 on the surface of the cells.

In any of the methods comprising the measurement of PD-L1 expression in a cancer tissue sample, however, it should be understood that the step comprising the provision of a cancer tissue sample obtained from a subject is an optional step. It should also be understood that in certain embodiments the “measuring” or “assessing” step to identify, or determine the number or proportion of, cells in the cancer tissue sample that express PD-L1 on the cell surface is performed by a transformative method of assaying for PD-L1 expression, for example by performing a reverse transcriptase-polymerase chain reaction (RT-PCR) assay or an immunohistochemical (IHC) assay. In some embodiments, no transformative step is involved and PD-L1 expression is assessed by, for example, reviewing a report of test results from a laboratory. In certain embodiments, the steps of the methods up to, and including, assessing PD-L 1 expression provides an intermediate result that may be provided to a physician or other healthcare provider for use in selecting a suitable subject for treatment. In certain embodiments, the steps that provide the intermediate result is performed by a medical practitioner or someone acting under the direction of a medical practitioner. In other embodiments, these steps are performed by an independent laboratory or by an independent person such as a laboratory technician.

In some embodiments, the proportion of cells that express PD-L1 is assessed by performing an assay to determine the presence of PD-L1 RNA. In some embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In some embodiments, the presence of PD-L1 polypeptide is determined by an IHC assay, an enzyme-linked immunosorbent assay (ELISA), in vivo imaging, or flow cytometry. In some embodiments, PD-L1 expression is determined by an RIC assay. See Chen et al., (2013) Clin. Cancer Res. 19(13): 3462-3473.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), may herald even greater use of these techniques in cancer research. Some of these molecular imaging systems allow clinicians to not only see where a cancer is located in the body, but also to visualize the expression and activity of specific molecules, cells, and biological processes that influence cancer behavior and/or responsiveness to therapeutic drugs (Condeelis and Weissleder, In vivo imaging in cancer, Cold Spring Harb. Perspect. Biol. 2(i2): a003848 (2010)). Antibody specificity, coupled with the sensitivity and resolution of PET, makes immunoPET imaging particularly attractive for monitoring and assaying expression of antigens in tissue samples (McCabe and Wu, Positive progress in immunoPET—not just a coincidence, Cancer Biother. Radiopharm. 25(3):253-61 (2010); Olafsen et al., ImmunoPET imaging of B-cell lymphoma using 124I-anti-CD20 scFv dimers (diabodies), Protein Eng. Des. Sel. 23(4):243-9 (2010)). In certain embodiments, PD-L1 expression is assayed by immunoPET imaging. In certain embodiments, the proportion of cells in a cancer tissue sample that express PD-L1 is assessed by performing an assay to determine the presence of PD-L1 polypeptide on the surface of cells in the cancer tissue sample. In certain embodiments, the cancer tissue sample is a formalin-fixed paraffin-embedded (FFPE) tissue sample. In other embodiments, the presence of PD-L1 polypeptide is determined by an IHC assay. In further embodiments, the IHC assay is performed using an automated process. In some embodiments, the IHC assay is performed using an anti-PD-L1 monoclonal antibody to bind to the PD-L1 polypeptide.

In some embodiments, an automated IHC method is used to assay the expression of PD-L1 on the surface of cells in FFPE tissue specimens. This disclosure provides methods for detecting the presence of human PD-L1 antigen in a cancer tissue sample, or quantifying the level of human PD-L1 antigen or the proportion of cells in the sample that express the antigen, which methods comprise contacting the test sample, and a negative control sample, with a monoclonal antibody that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In some embodiments, an automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a post primary blocking agent; incubating with NovoLink Polymer; adding a chromogen substrate and developing; and counterstaining with hematoxylin.

For assessing PD-L1 expression in cancer tissue samples, a pathologist may examine the number of membrane PD-L1+ cancer cells in each field under a microscope and mentally estimates the percentage of cells that are positive, then averages them to come to the final percentage. The different staining intensities may be defined as 0/negative, 1+/weak, 2+/moderate, and 3+/strong. Percentage values may be first assigned to the 0 and 3+ buckets, and then the intermediate 1+ and 2+ intensities may be considered. For highly heterogeneous tissues, the specimen may be divided into zones, and each zone may be scored separately and then combined into a single set of percentage values. The percentages of negative and positive cells for the different staining intensities are determined from each area and a median value is given to each zone. A final percentage value may be given to the tissue for each staining intensity category: negative, 1+, 2+, and 3+. The sum of all staining intensities may be 100%.

Staining is also assessed in cancer-infiltrating inflammatory cells such as macrophages and lymphocytes. In most cases macrophages serve as an internal positive control since staining is observed in a large proportion of macrophages. While not required to stain with 3+ intensity, an absence of staining of macrophages may be taken into account to rule out any technical failure. Macrophages and lymphocytes may be assessed for plasma membrane staining and only recorded for all samples as being positive or negative for each cell category. Staining is also characterized according to an outside/inside cancer immune cell designation. “Inside” means the immune cell is within the cancer tissue and/or on the boundaries of the cancer region without being physically intercalated among the cancer cells. “Outside” means that there is no physical association with the cancer, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. In some embodiments, the histoscore may be calculated as follows: Histoscore=[(% cancer×1 (low intensity))+(% cancer×2 (medium intensity))+(% cancer×3 (high intensity)]

In some embodiment, to determine the histoscore, the pathologist may estimate the percentage of stained cells in each intensity category within a specimen. Because expression of most biomarkers is heterogeneous, the histoscore can be a truer representation of the overall expression. The final histoscore range is 0 (no expression) to 300 (maximum expression).

In some embodiments, a means of quantifying PD-L1 expression in a cancer is to determine the adjusted inflammation score (AIS) score defined as the density of inflammation multiplied by the percent PD-L1 expression by cancer-infiltrating inflammatory cells. Taube et al., Colocalization of inflammatory response with B7-h1 expression in human melanocytic lesions supports an adaptive resistance mechanism of immune escape, Sci. Transl. Med. 4(127):127ra37 (2012)).

In some embodiments, a means of quantifying PD-L1 expression in a cancer is to determine the Combined Positive Score (CPS), which as described above, is the number of PD-L1 staining cells (tumor cells, lymphocytes, macrophages) divided by the total number of viable tumor cells, multiplied by 100. For some therapeutic treatments, a tumor sample is considered to have PD-L1 expression if CPS≥1. For example, a CPS≥10 is required for a subject to be eligible for certain PD-1 or PD-L1 inhibitor therapies, such as subjects with urothelial cancer (bladder cancer), esophageal squamous cell carcinoma (ESCC), or triple-negative breast cancer being treated with pembrolizumab.

In some embodiments, a means of quantifying PD-L1 expression in a cancer is to determine the Tumor Proportion Score (TPS), which as described above, is the percentage of viable tumor cells showing partial or complete membrane staining at any intensity. For some therapeutic treatments, a tumor sample is considered to have PD-L1 expression if TPS≥1% and high PD-L1 expression if TPS≥50%.

In some embodiments, a means for quantifying PD-L1 expression in a cancer is to determine a Tumor Cell (TC) score. For some therapeutic treatments, a tumor sample is considered to have PD-L1 expression if TC≥50%.

In some embodiments, a means for quantifying PD-L1 expression in a cancer is to determine a Tumor-Infiltrating Immune Cell (IC) score. For some therapeutic treatments, a tumor sample is considered to have PD-L1 expression if a specimen contains PD-L1 staining of any intensity in tumor infiltrating immune cells occupying ≥5% of tumor area.

In some embodiments, a means of quantifying PD-L1 expression in a cancer is the Agilent (Dako) PD-L1 IHC 223 pharmDx Assay™, a description of which may be found in at least one of the following: 1) Physician Labeling, Dako PD-L1 IHC 22C3 pharmDx, Dako North America, Inc., Carpinteria, CA; 2) Keytruda package insert (2021) Merck & Co., Inc., Kenilworth, NJ; 3) PD-L1 IHC 22C3 pharmDx Instructions for Use (2020) Dako, Agilent Pathology Solutions, Carpinteria, CA; 4) Garon E B, Rizvi N A, Hui R, et al. Pembrolizumab for the treatment of non-small-cell lung cancer, N. Engl. J. Med. 372(21):2018-2028 (2015); and 5) Roach C, Zhang N, Corigliano E, et al. Development of a companion diagnostic PD-L1 immunohistochemistry assay for pembrolizumab therapy in non-small-cell lung cancer, Appl Immunohistochem Mol. Morphol. 24:392-397 (2016).

In some embodiments, a means of quantifying PD-L1 expression in a cancer is the Agilent (Dako) PD-L1 IHC 28-8 pharmDx Assay™, a description of which may be found in at least one of the following: 1) Physician Labeling, Dako PD-L1 IHC 28-8 pharmDx (2020) Dako North America, Inc., Carpinteria, CA; 2) OPDIVO package insert (2021) Bristol Myers Squibb, New York, NY; 3) PD-L1 IHC 28-8 pharm Dx: Interpretation Manual (2021), Dako, Agilent Pathology Solutions, Carpinteria, CA; and 4) Phillips T, Simmons P, Inzunza H D, Cogswell J, Novotny J Jr, Taylor C, et al. Development of an automated PD-L1 immunohistochemistry (IHC) assay for non-small cell lung cancer, Appl. Immunohistochem. Mol. Morphol. 23:541-9 (2015).

In some embodiments, a means of quantifying PD-L1 expression in a cancer is the Agilent (Dako) PD-L1 IHC 73-10 Assay™, a description of which may be found in at least one of the following: 1) Hans, J. G. et al. PD-L1 Immunohistochemistry Assay Comparison Studies in Non-Small Cell Lung Cancer: Characterization of the 73-10 Assay, J. Thoracic Oncology 15:1306-1316 (2020); and 2) Bavencio package insert (2021) EMD Serono, Inc. Rockland, MA and Pfizer Inc., New York, NY.

In some embodiments, a means of quantifying PD-L1 expression in a cancer is the Ventana PD-L1 (SP142) Assay™, a description of which may be found in at least one of the following: 1) Physician Labeling: Ventana PD-L1 (SP142) Assay (2020) Ventana Medical Systems, Inc. and Roche Diagnostics International, Inc.; 2) Tecentriq package insert (2021) Genentech, Inc., South San Francisco, CA; 3) Ventana PD-L1 (SP142) Assay: Interpretation Guide (2019) Ventana Medical Systems, Inc. and Roche Diagnostics International, Inc.; and 4) Vennapusa et al., Development of a PD-L1 Complementary Diagnostic Immunochemistry Assay (SP142) for Atezolizumab, Appl. Immunohistochem. Mol. Morphol. 27:92-100 (2019).

In some embodiments, a means of quantifying PD-L1 expression in a cancer is the Ventana PD-L1 (SP263) Assay™, a description of which may be found in at least one of the following: 1) Physician Labeling: Ventana PD-L1 (SP263) Assay (2017) Ventana Medical Systems, Inc., Tucson, AZ; 2) Imfinzi package insert (2021), AstraZeneca Pharmaceuticals LP, Wilmington, DE; and 3) Ventana PD-L1 (SP263) Assay Staining: Interpretation Guide (2019) Roche Diagnostics GmbH, Munich, DE.

Table 3 below provides a summary of the above assays, the drugs for which they may be used, and indications for those treatments as currently approved in the US. Some of the combination therapies provided herein utilize the drugs in the indications as listed in Table 3, together with the corresponding assay to determine PD-L1 expression levels.

TABLE 3
Summary of PD-L1 Diagnostic Assays

| Diagnostic- | | |
|---|---|---|
| Antibody | Drug | Indication |
| 22C3- | Pembrolizumab | NSCLC, urothelial, CHL, |
| Dako/ | (KEYTRUDA)-Merck | melanoma, HNSCC, gastric/ |
| Agilent | | GEJ |
| 28-8- | Nivolumab | CRC, melanoma, |
| Dako/ | (OPDIVO)- | nsNSCLC, renal cancer, |
| Agilent | Bristol Myers | advanced liver, renal and |
| | Squibb | urothelical cancers, HNSCC, |
| 73-10- | Avelumab | Urothelial cancer, merkel |
| Dako/ | (BAVENCIO)- | cell carcinoma |
| Agilent | EMD | |
| | Serono/Pfizer | |
| SP142- | Atezolizumab | NSCLC, urothelial |
| Ventana | (TECENTRIQ)-Genentech | cancer |
| SP263- | Durvalumab | Advanced NSCLC, |
| Ventana | (IMFINZI)-AstraZeneca | urothelial cancer |

Additionally, O'Malley et al., Immunohistochemical detection of PD-L1 among diverse human neoplasms in a reference laboratory: observations based upon 62,896 cases, Modern Pathology 32:929-942 (2019), provides a description evaluating PD-L1 expression using antibody clones 22C3, 28-8, SP142, or SP263, in various types of cancers.

The present disclosure also provides methods of manufacturing the combination therapies for various uses as described herein. The combination therapy can be included in a container, pack, kit, or dispenser together with instructions for administration.

Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications can be practiced within the scope of the appended claims.

### EXAMPLES

The invention is further described in the following examples, which do not limit the scope of the invention described in the claims.

### Example 1: Murine Tumor Models with Concomitant or Staggered Dosing of a SEA-CD40 Surrogate and/or an Anti-Mpd-1 Surrogate Antibody

Mouse models have been proven to be very useful in assessing efficacy and mechanisms of cancer therapeutics. Study of SEA-CD40 in murine cancer models has been difficult because SEA-CD40 does not recognize murine CD40. Therefore, to assess the activity of the non-fucosylated anti-CD40 antibodies, syngeneic murine tumor models were developed. The murine functional equivalents of human IgG1 and human FcγRIII/CD16 are murine IgG2a and murine FcγRIV, respectively, and binding of murine IgG2a to murine FcγRIV mediates antibody-dependent cellular cytotoxicity (ADCC). See, e.g., Bruhns, Blood 119:5640-5649 (2012) and Nimmeriahn et al., Immunity 23:41-51 (2005). The rat antibody 1C10 was used to generate a surrogate of SEA-CD40. See, e.g., Heath et al., Eur. J. Immunol. 24:1828-1834 (1994). Briefly, the VL and VH gene fragments of a rat monoclonal antibody can recognize murine CD40. The 1C10 antibody were cloned in-frame 5′ to murine Ckappa and murine IgG2a CH1-CH2-CH3 fragments, respectively. Expression of the resulting genes in CHO cells generated a chimeric 1C10 antibody with rat VL and VH domains and murine light and heavy chain domains of the IgG2a isotype (mIgG2a 1C10). mIgG2a 1C10 was expressed in the presence of 2-fluorofucose in the CHO cell growth medium using the methods described in U.S. Patent Application Publication No. US 2017/0333556 A1, to generate a non-fucosylated form of mIgG2a 1C10 (mIgG2a SEA 1C10, or SEA-m1C10).

The single agent activity of SEA-m1C10 or an anti-mPD-1 surrogate antibody (“anti-PD1”), and a combination thereof, were investigated in solid and syngeneic tumor models. Based on SEA-CD40's mechanism (e.g., enhanced activation of antigen presenting cells, and subsequent induction of an amplified anti-tumor T cell response), SEA-m1C10 was administered prior to the initial treatment with the anti-mPD-1 surrogate antibody.

Stock solutions of the antibodies were diluted to the appropriate concentration and then injected into animals in a volume of 100 μl. Final dosages were 1 mg/kg for SEA-m1C10, and 1 mg/kg for the anti-mPD-1 surrogate antibody. Tumor length, tumor width, and mouse weight were measured throughout the experimental period, and tumor volume was calculated. Euthanasia was performed when tumor volume of a mouse reached 1000 mm³.

### CT26 Colon Cancer Model

The combinatorial activity of the SEA-m1C10 antibody and the anti-mPD-1 surrogate antibody was tested in a CT26 colon cancer model, which is responsive to anti-mPD-1 surrogate antibody treatment. BALB/c mice were implanted with the CT26 syngeneic tumor cell line subcutaneously in the flank of mice on day 0. When the mean tumor size (measured using the formula: Volume (mm³)=0.5*Length*Width², wherein length is the longer dimension and width is the shorter dimension) reached 100 mm³, mice were randomly placed into a control group G1 and three treatment groups G2-G4 (5 mice per group).

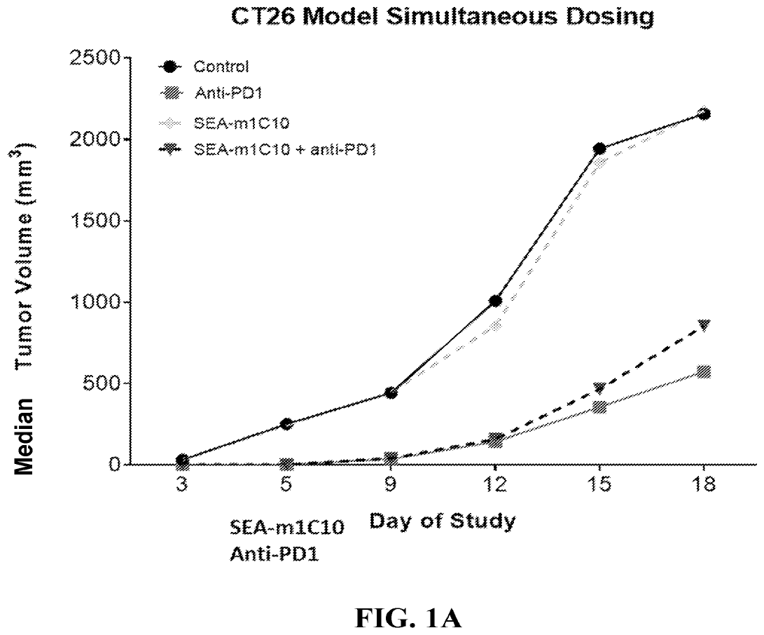

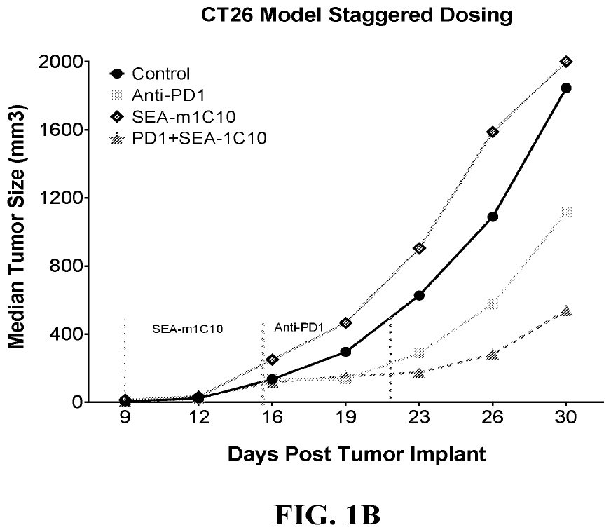

In one experiment, the treatment group mice were administered intraperitoneally with either a single agent (the anti-mPD-1 surrogate antibody (G2) or SEA-m1C10 (G3)), or a combination thereof (G4) on the same day. The administration frequency was once every three days for a total of three treatments. The control group mice (G1) were untreated. Median tumor volume of the mice are shown in FIG. 1A.

Alternatively, mice in treatment groups G3 and G4 were administered with 3 doses of SEA-m1C10 three days apart (e.g., within the period from day 9 to day 15). On the last day of SEA-m1C10 treatment (e.g., on day 15), the first dose of the anti-mPD-1 surrogate antibody was administered to the G2 and G4 group mice, which then received 2 additional doses three days apart (e.g., within the period from day 15 to day 21). The control group mice (G1) were untreated. Median tumor volume of the mice are shown in FIG. 1B.

The results showed that SEA-m1C10 did not exhibit any anti-tumor effect when administered alone. As shown in FIG. 1A, no combinatorial activity, or even an antagonistic activity, was observed when SEA-m1C10 and the anti-mPD-1 surrogate antibody were administered concomitantly. However, the anti-tumor activity was enhanced when SEA-m1C10 was administered in a staggered manner with the anti-mPD-1 surrogate antibody (FIG. 1B). The above results indicate that the timing of administration of these agents may be important to achieve a therapeutic benefit for certain cancer.

### A20 Disseminated Lymphoma Model

The combinatorial activity of the SEA-m1C10 antibody and the anti-mPD-1 surrogate antibody was also tested in an A20 lymphoma model. BALB/c mice were injected intravenously with A20 cells which established a disseminated lymphoma in about 2-4 weeks. A20 disseminated lymphoma model was initiated in immune-competent female BALB/c mice by injecting 1×10⁶ A20 cells per mouse intravenously (IV). Mice were randomly placed into a control group G1 and three treatment groups G2-G4 (6 mice per group).

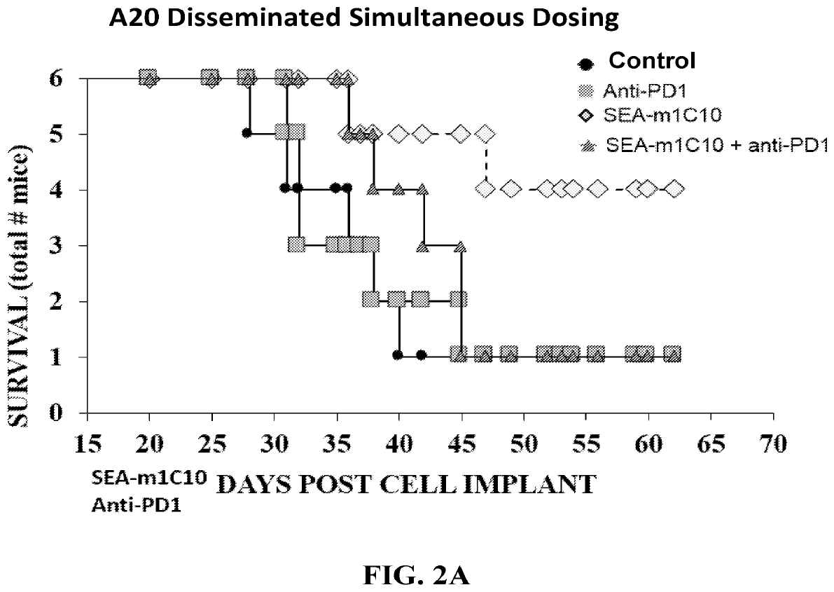

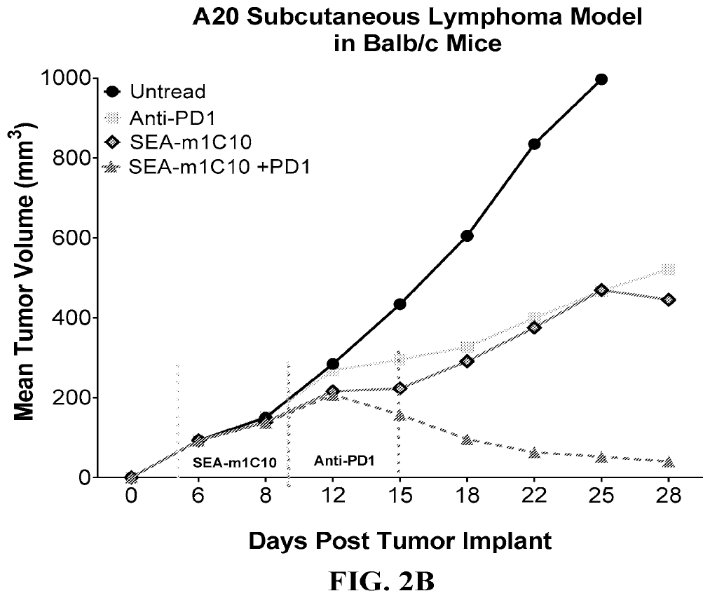

In one experiment, the treatment group mice were administered with antibodies at 3 mg/kg intraperitoneally (i.p.) on a q3d×3 schedule (once every three days for a total of three treatments) starting on day 7 post tumor cell inoculation. The control group mice were untreated. All mice were monitored for weight loss and symptoms of tumor burden, e.g., ascites in the peritoneum. The tumor burden was further verified after sacrificing the mice. FIG. 2A shows the survival curves of the control group mice (G1), mice treated with the anti-mPD-1 surrogate antibody (G2), SEA-m1C10 (G3), and a combination thereof on the same days (G4).

In a different experiment, an A20 lymphoma model was established via subcutaneous methodology. Specifically, tumors were allowed to grow to about 100 mm³ and mice in treatment groups G3 and G4 were administered with 3 doses of SEA-m1C10 three days apart (e.g., within the period from day 4 to day 10). On the last day of SEA-m1C10 treatment, the first dose of the anti-mPD1 surrogate antibody was administered (e.g., on day 10) to the G2 and G4 group mice, which then received 2 additional doses three days apart (e.g., within the period from day 10 to day 16). The control group mice (G1) were untreated. Mean tumor volume of the mice are shown in FIG. 2B.

As shown in FIG. 2A, mice treated with SEA-m1C10 (G3) significantly extended animal survival, as compared to that of the control group mice (G1). Further, concomitant administration of SEA-m1C10 and the anti-mPD1 surrogate antibody in a disseminated model of A20 did not exhibit any anti-tumor activity and even appeared antagonistic when administered together. In contrast, as shown in FIG. 2B, the anti-tumor activity was enhanced when SEA-m1C10 was administered in a staggered manner with the anti-mPD-1 surrogate antibody. For example, while both the anti-mPD-1 surrogate antibody (G2) and SEA-m1C10 (G3) delayed tumor progression with 4/6 and 5/6 animals achieving a complete response (CR), respectively, the combinatorial activity of these two agents was striking, driving complete tumor regression in 6/6 animals (G4).

### RENCA Renal Cell Carcinoma Model

The combinatorial activity of the SEA-m1C10 antibody with the anti-mPD1 surrogate antibody was also tested in a subcutaneous RENCA renal cell syngeneic model. BALB/c mice were implanted with the RENCA syngeneic tumor cell line subcutaneously in the flank of mice on day 0. When the mean tumor size (measured using the formula: Volume (mm³)=0.5*Length*Width², wherein length is the longer dimension and width is the shorter dimension) reached 100 mm³, mice were randomly placed into a control group G1 and three treatment groups G2-G4 (5 mice per group).

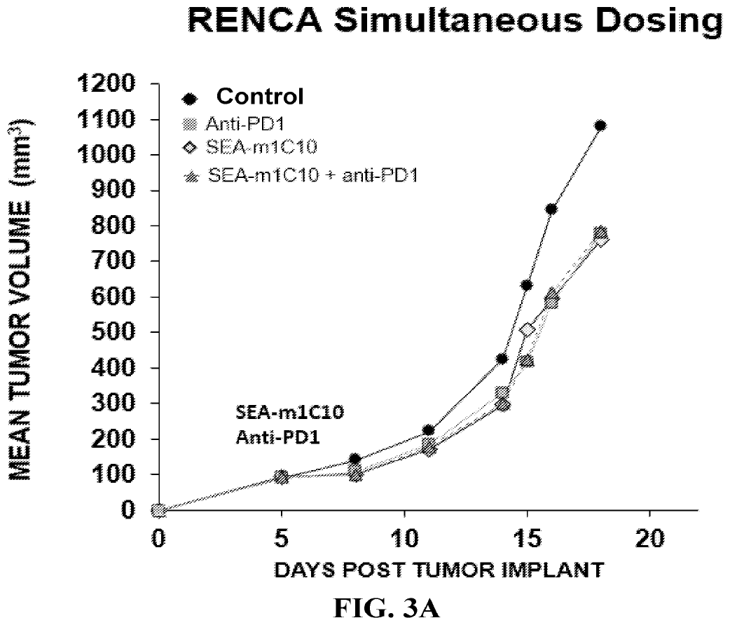

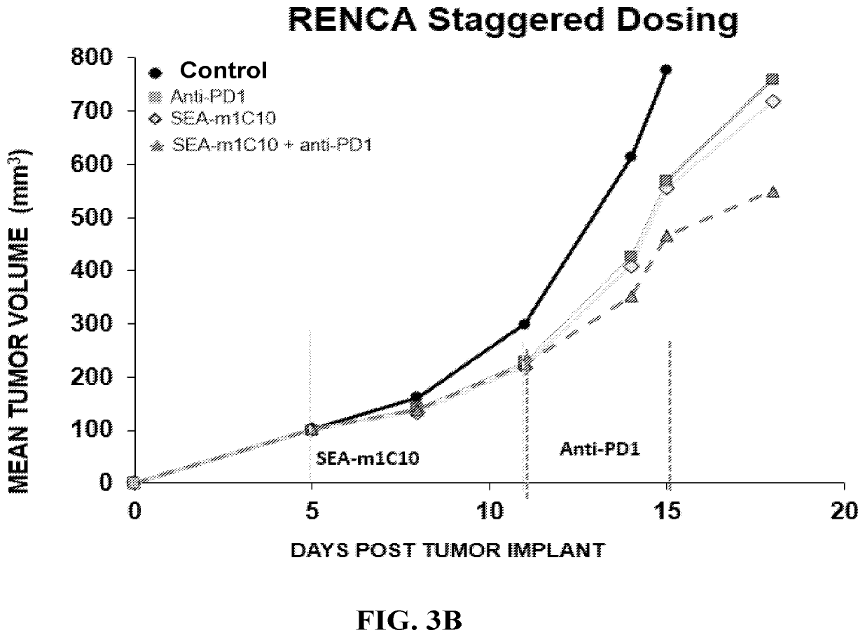

In one experiment, the treatment group mice were administered intraperitoneally with either a single agent (the anti-mPD-1 surrogate antibody (G2) or SEA-m1C10 (G3)), or a combination thereof (G4) on the same day. The administration frequency was once every three days for a total of three treatments. The control group mice (G1) were untreated. Mean tumor volume of the mice are shown in FIG. 3A.

Alternatively, mice in treatment groups G3 and G4 were administered with 3 doses of SEA-m1C10 three days apart (e.g., within the period from day 5 to day 11). On the last day of SEA-m1C10 treatment (e.g., on day 11), the first dose of the anti-mPD-1 surrogate antibody was administered to the G2 and G4 group mice, which then received 2 additional doses three days apart (e.g., within the period from day 9 to day 15). The control group mice (G1) were untreated. Mean tumor volume of the mice are shown in FIG. 3B.

As shown in FIG. 3A, the results showed that concomitant administration of SEA-m1C10 and the anti-mPD-1 surrogate antibody did not exhibit any anti-tumor activity and even appeared antagonistic when administered together. As shown in FIG. 3B, while both SEA-m1C10 and the anti-mPD-1 surrogate antibody delayed tumor progression, the combinatorial activity of these two agents administered in a staggered manner improved anti-tumor activity and resulted in tumor growth delay.

### Example 2: Treating Melanoma Patients with a Combination of SEA-CD40 and Pembrolizumab

Treatment of melanoma is being assessed with the combination of SEA-CD40 and pembrolizumab in a phase 2 clinical trial. Some patients with melanoma are treated with a combination of SEA-CD40 and pembrolizumab. SEA-CD40 is administered at a dose of 10 μg/kg on day 1 and day 21 of each 42-day cycle. Pembrolizumab is administered on day 8 of each 42-day cycle at 400 mg.

Some patients with melanoma are treated with a combination of SEA-CD40 and pembrolizumab. SEA-CD40 is administered at a dose of 10 μg/kg on day 1 and day 22 of each 42-day cycle. Pembrolizumab is administered on day 8 of each 42-day cycle at 400 mg.

Some patients with relapsed and/or refractory melanoma are treated with a combination of SEA-CD40 and pembrolizumab. SEA-CD40 is administered as an IV infusion at a dose of 10 μg/kg on day 1 and day 21 of each 42-day cycle. Pembrolizumab is administered IV on day 8 of each 42-day cycle at 400 mg.

Some patients with relapsed and/or refractory melanoma are treated with a combination of SEA-CD40 and pembrolizumab. SEA-CD40 is administered as an IV infusion at a dose of 10 g/kg on day 1 and day 22 of each 42-day cycle. Pembrolizumab is administered IV on day 8 of each 42-day cycle at 400 mg.

Some patients with melanoma are treated with a combination of SEA-CD40 and pembrolizumab. SEA-CD40 is administered at a dose of 10 μg/kg on day 1 of each 21-day cycle. Pembrolizumab is administered on day 8 of each 21-day cycle.

Some patients with relapsed and/or refractory melanoma are treated with a combination of SEA-CD40 and pembrolizumab. SEA-CD40 is administered at a dose of 10 μg/kg on day 1 of each 21-day cycle. Pembrolizumab is administered on day 8 of each 21-day cycle.

Some patients with melanoma are treated with a combination of SEA-CD40 and pembrolizumab. SEA-CD40 is administered at a dose of 10 ag/kg on day 1 of each 21-day cycle. Pembrolizumab is not administered on the first 21-day cycle, and is administered on day 8 of each 21-day cycle starting from the second 21-day cycle.

Some patients with refractory/relapsed melanoma are treated with a combination of SEA-CD40 and pembrolizumab. SEA-CD40 is administered at a dose of 10 μg/kg on day 1 of each 21-day cycle. Pembrolizumab is not administered on the first 21-day cycle, and is administered on day 8 of each 21-day cycle starting from the second 21-day cycle.

Some patients with melanoma are treated with a combination of SEA-CD40 and pembrolizumab. SEA-CD40 is administered in 21-day cycles and pembrolizumab is administered in 42-day cycles. SEA-CD40 is administered at a dose of 10 μg/kg on day 1 of each 21-day cycle. Pembrolizumab is not administered on the first 42-day cycle, and is administered on day 8 of each 42-day cycle starting from the second 42-day cycle.

Some patients with refractory/relapsed melanoma are treated with a combination of SEA-CD40 and pembrolizumab. SEA-CD40 is administered in 21-day cycles and pembrolizumab is administered in 42-day cycles. SEA-CD40 is administered at a dose of 10 μg/kg on day 1 of each 21-day cycle. Pembrolizumab is not administered on the first 42-day cycle, and is administered on day 8 of each 42-day cycle starting from the second 42-day cycle.

Primary outcome measure is confirmed Objective Response Rate (ORR): the proportion of subjects who achieve a confirmed complete response (CR) or partial response (PR) according to RECIST v1.1 per investigator assessment (Eisenhauer 2009). The timeframe is the duration of treatment, approximately two years.

Secondary outcome measure is type, incidence, severity, seriousness, and relatedness of adverse events (AEs). The incidence of adverse events (AEs) include any untoward medical occurrence in a subject or clinical investigational subject administered a medicinal product and which does not necessarily have a causal relationship with this treatment. The time frame is from the start of treatment to 30-37 days after last dose, approximately 2 years.

Secondary outcome measure also includes type, incidence, and severity of laboratory abnormalities. The incidence of laboratory abnormalities are to be summarized using descriptive statistics. The time frame is from the start of treatment to 30-37 days after last dose, approximately 2 years.

Secondary outcome measure also includes frequency of treatment interruptions, dose reductions, and treatment discontinuations due to AEs. The incidence of dose alterations is to be summarized using descriptive statistics. The time frame is the duration of treatment, approximately two years.

Secondary outcome measure also includes disease control rate (DCR; confirmed CR, PR, and stable disease [SD]) per investigator assessment. Disease control rate (DCR) per investigator assessment is the proportion of subjects who achieve a confirmed CR or PR according to RECIST v1.1 as assessed by the investigator or meet the stable disease (SD) criteria at least once after start of study treatment at a minimum interval of 5 weeks. The time frame is from the start of treatment until completion of response assessment, approximately 4 years.

Secondary outcome measure also includes duration of response (DOR) per investigator assessment: the time from the first documentation of objective response (CR or PR that is subsequently confirmed) to the first documentation of progressive disease (PD) or death due to any cause. The time frame is from the start of treatment until completion of response assessment, approximately 4 years.

Secondary outcome measure also includes progression-free survival (PFS) per investigator assessment: the time from the start of study treatment to the first documentation of PD by RECIST v1.1 or death due to any cause. The time frame is from the start of treatment until completion of response assessment, approximately 4 years.

Secondary outcome measure also includes overall survival (OS): the time from the start of study treatment to date of death due to any cause. The time frame is duration of study, approximately 4 years.

Eligibility criteria for relapsed and/or refractory metastatic melanoma may include: uveal/ocular melanoma is excluded; must have progressed on treatment with an anti-PD-(L)1 mAb; able to provide archival tumor tissue from locations not radiated prior to biopsy; Eastern Cooperative Oncology Group (ECOG) Performance Status score of 0 or 1; and Measurable disease per RECIST v1.1 at baseline.

Exclusion criteria may include: history of another malignancy within 3 years of first dose of study drug; active central nervous system (CNS) metastases and/or carcinomatous meningitis; previous exposure to CD40-targeted therapy; currently on chronic systemic steroids in excess of physiologic replacement; has had an allogeneic tissue/solid organ transplant; history of autoimmune disease that has required systemic treatment in the past 2 years.

Eligible patients with relapsed/refractory melanoma are treated with a combination of SEA-CD40 and pembrolizumab. SEA-CD40 is administered as an IV infusion at a dose of 10 g/kg on day 1 and day 21 of each 42-day cycle. Pembrolizumab is administered IV on day 8 of each 42-day cycle at 400 mg. Primary and secondary outcomes are assessed as described above.

### Example 3: Treating Metastatic Uveal Melanoma Patients with a Combination of SEA-CD40 and Pembrolizumab

Treatment of metastatic uveal melanoma is being assessed with the combination of SEA-CD40 and pembrolizumab in a phase 2 clinical trial. Patients with metastatic uveal melanoma are treated with a combination of SEA-CD40 and pembrolizumab. SEA-CD40 is administered at a dose of 10 μg/kg on day 1 and day 21 of each 42-day cycle. Pembrolizumab is administered on day 8 of each 42-day cycle at 400 mg.

Some patients with metastatic uveal melanoma are treated with a combination of SEA-CD40 and pembrolizumab. SEA-CD40 is administered at a dose of 10 μg/kg on day 1 and day 22 of each 42-day cycle. Pembrolizumab is administered on day 8 of each 42-day cycle at 400 mg.

Some patients with metastatic uveal melanoma are treated with a combination of SEA-CD40 and pembrolizumab. SEA-CD40 is administered at a dose of 10 μg/kg on day 1 of each 21-day cycle. Pembrolizumab is administered on day 8 of each 21-day cycle.

Some patients with metastatic uveal melanoma are treated with a combination of SEA-CD40 and pembrolizumab. SEA-CD40 is administered at a dose of 10 μg/kg on day 1 of each 21-day cycle. Pembrolizumab is not administered on the first 21-day cycle, and is administered on day 8 of each 21-day cycle starting from the second 21-day cycle.

Some patients with metastatic uveal melanoma are treated with a combination of SEA-CD40 and pembrolizumab. SEA-CD40 is administered in 21-day cycles and pembrolizumab is administered in 42-day cycles. SEA-CD40 is administered at a dose of 10 μg/kg on day 1 of each 21-day cycle. Pembrolizumab is not administered on the first 42-day cycle, and is administered on day 8 of each 42-day cycle starting from the second 42-day cycle.

Primary outcome measure is confirmed Objective Response Rate (ORR). the proportion of subjects who achieve a confirmed complete response (CR) or partial response (PR) according to RECIST v1.1 per investigator assessment (Eisenhauer 2009). The timeframe is the duration of treatment, approximately two years.

Secondary outcome measure is type, incidence, severity, seriousness, and relatedness of adverse events (AEs). The incidence of adverse events (AEs) include any untoward medical occurrence in a subject or clinical investigational subject administered a medicinal product and which does not necessarily have a causal relationship with this treatment. The time frame is from the start of treatment to 30-37 days after last dose, approximately 2 years.

Secondary outcome measure also includes type, incidence, and severity of laboratory abnormalities. The incidence of laboratory abnormalities are to be summarized using descriptive statistics. The time frame is from the start of treatment to 30-37 days after last dose, approximately 2 years.

Secondary outcome measure also includes frequency of treatment interruptions, dose reductions, and treatment discontinuations due to AEs. The incidence of dose alterations is to be summarized using descriptive statistics. The time frame is the duration of treatment, approximately two years.

Secondary outcome measure also includes disease control rate (DCR; confirmed CR, PR, and stable disease [SD]) per investigator assessment. Disease control rate (DCR) per investigator assessment is the proportion of subjects who achieve a confirmed CR or PR according to RECIST v1.1 as assessed by the investigator or meet the stable disease (SD) criteria at least once after start of study treatment at a minimum interval of 5 weeks. The time frame is from the start of treatment until completion of response assessment, approximately 4 years.

Secondary outcome measure also includes duration of response (DOR) per investigator assessment: the time from the first documentation of objective response (CR or PR that is subsequently confirmed) to the first documentation of progressive disease (PD) or death due to any cause. The time frame is from the start of treatment until completion of response assessment, approximately 4 years.

Secondary outcome measure also includes progression-free survival (PFS) per investigator assessment: the time from the start of study treatment to the first documentation of PD by RECIST v1.1 or death due to any cause. The time frame is from the start of treatment until completion of response assessment, approximately 4 years.

Secondary outcome measure also includes overall survival (OS): the time from the start of study treatment to date of death due to any cause. The time frame is duration of study, approximately 4 years.

Eligibility criteria for metastatic uveal melanoma may include: must not have received prior treatment for advanced or metastatic disease except for prior adjuvant/neoadjuvant immunotherapy; no prior regional, liver-directed therapy; able to provide archival tumor tissue from locations not radiated prior to biopsy; Eastern Cooperative Oncology Group (ECOG) Performance Status score of 0 or 1; and Measurable disease per RECIST v1.1 at baseline.

Exclusion criteria may include: history of another malignancy within 3 years of first dose of study drug; active central nervous system (CNS) metastases and/or carcinomatous meningitis; previous exposure to CD40-targeted therapy; currently on chronic systemic steroids in excess of physiologic replacement; has had an allogeneic tissue/solid organ transplant; history of autoimmune disease that has required systemic treatment in the past 2 years.

Eligible patients with metastatic uveal melanoma are treated with a combination of SEA-CD40 and pembrolizumab. SEA-CD40 is administered as an IV infusion at a dose of 10 μg/kg on day 1 and day 21 of each 42-day cycle. Pembrolizumab is administered IV on day 8 of each 42-day cycle at 400 mg. Primary and secondary outcomes are assessed as described above.

Eligible patients with metastatic uveal melanoma are treated with a combination of SEA-CD40 and pembrolizumab. SEA-CD40 is administered as an IV infusion at a dose of 10 g/kg on day 1 and day 22 of each 42-day cycle. Pembrolizumab is administered IV on day 8 of each 42-day cycle at 400 mg. Primary and secondary outcomes are assessed as described above.

### Example 4: Treating Metastatic PD-(L)1-Naïve Melanoma with a Combination of SEA-CD40 and Pembrolizumab

Treatment of metastatic PD-(L)1-naïve melanoma is being assessed with the combination of SEA-CD40 and pembrolizumab in a phase 2 clinical trial. Patients with metastatic PD-(L)1-naïve melanoma are treated with a combination of SEA-CD40 and pembrolizumab. SEA-CD40 is administered at a dose of 10 μg/kg on day 1 and day 21 of each 42-day cycle. Pembrolizumab is administered on day 8 of each 42-day cycle at 400 mg.

Patients with metastatic PD-(L)1-naïve melanoma are treated with a combination of SEA-CD40 and pembrolizumab. SEA-CD40 is administered at a dose of 10 μg/kg on day 1 and day 22 of each 42-day cycle. Pembrolizumab is administered on day 8 of each 42-day cycle at 400 mg.

Some patients with metastatic PD-(L)1-naïve melanoma are treated with a combination of SEA-CD40 and pembrolizumab. SEA-CD40 is administered at a dose of 10 μg/kg on day 1 of each 21-day cycle. Pembrolizumab is administered on day 8 of each 21-day cycle.

Some patients with metastatic PD-(L)1-naïve melanoma are treated with a combination of SEA-CD40 and pembrolizumab. SEA-CD40 is administered at a dose of 10 μg/kg on day 1 of each 21-day cycle. Pembrolizumab is not administered on the first 21-day cycle, and is administered on day 8 of each 21-day cycle starting from the second 21-day cycle.

Some patients with metastatic PD-(L)1-naïve melanoma are treated with a combination of SEA-CD40 and pembrolizumab. SEA-CD40 is administered in 21-day cycles and pembrolizumab is administered in 42-day cycles. SEA-CD40 is administered at a dose of 10 g/kg on day 1 of each 21-day cycle. Pembrolizumab is not administered on the first 42-day cycle, and is administered on day 8 of each 42-day cycle starting from the second 42-day cycle.

Some primary outcome measure is confirmed Objective Response Rate (ORR): the proportion of subjects who achieve a confirmed complete response (CR) or partial response (PR) according to RECIST v1.1 per investigator assessment (Eisenhauer 2009). The timeframe is the duration of treatment, approximately two years.

Secondary outcome measure is type, incidence, severity, seriousness, and relatedness of adverse events (AEs). The incidence of adverse events (AEs) include any untoward medical occurrence in a subject or clinical investigational subject administered a medicinal product and which does not necessarily have a causal relationship with this treatment. The time frame is from the start of treatment to 30-37 days after last dose, approximately 2 years.

Secondary outcome measure also includes type, incidence, and severity of laboratory abnormalities. The incidence of laboratory abnormalities are to be summarized using descriptive statistics. The time frame is from the start of treatment to 30-37 days after last dose, approximately 2 years.

Secondary outcome measure also includes frequency of treatment interruptions, dose reductions, and treatment discontinuations due to AEs. The incidence of dose alterations is to be summarized using descriptive statistics. The time frame is the duration of treatment, approximately two years.

Secondary outcome measure also includes disease control rate (DCR; confirmed CR, PR, and stable disease [SD]) per investigator assessment. Disease control rate (DCR) per investigator assessment is the proportion of subjects who achieve a confirmed CR or PR according to RECIST v1.1 as assessed by the investigator or meet the stable disease (SD) criteria at least once after start of study treatment at a minimum interval of 5 weeks. The time frame is from the start of treatment until completion of response assessment, approximately 4 years.

Secondary outcome measure also includes duration of response (DOR) per investigator assessment: the time from the first documentation of objective response (CR or PR that is subsequently confirmed) to the first documentation of progressive disease (PD) or death due to any cause. The time frame is from the start of treatment until completion of response assessment, approximately 4 years.

Secondary outcome measure also includes progression-free survival (PFS) per investigator assessment: the time from the start of study treatment to the first documentation of PD by RECIST v1.1 or death due to any cause. The time frame is from the start of treatment until completion of response assessment, approximately 4 years.

Secondary outcome measure also includes overall survival (OS): the time from the start of study treatment to date of death due to any cause. The time frame is duration of study, approximately 4 years.

Eligibility criteria for metastatic PD-(L)1-naïve melanoma may include: uveal/ocular melanoma is excluded; must not have received prior treatment for advanced or metastatic disease except for prior adjuvant/neoadjuvant immunotherapy; participants with BRAF mutation, prior BRAF/MEK targeted therapy is allowed if completed 4 weeks prior to first dose of study treatment no prior regional; able to provide archival tumor tissue from locations not radiated prior to biopsy; Eastern Cooperative Oncology Group (ECOG) Performance Status score of 0 or 1; and Measurable disease per RECIST v1.1 at baseline.

Exclusion criteria may include: history of another malignancy within 3 years of first dose of study drug; active central nervous system (CNS) metastases and/or carcinomatous meningitis; previous exposure to CD40-targeted therapy; currently on chronic systemic steroids in excess of physiologic replacement; has had an allogeneic tissue/solid organ transplant; history of autoimmune disease that has required systemic treatment in the past 2 years.

Eligible patients with metastatic PD-(L)1-naïve melanoma are treated with a combination of SEA-CD40 and pembrolizumab. SEA-CD40 is administered as an IV infusion at a dose of 10 μg/kg on day 1 and day 21 of each 42-day cycle. Pembrolizumab is administered IV on day 8 of each 42-day cycle at 400 mg. Primary and secondary outcomes are assessed as described above.

Eligible patients with metastatic PD-(L)1-naïve melanoma are treated with a combination of SEA-CD40 and pembrolizumab. SEA-CD40 is administered as an IV infusion at a dose of 10 μg/kg on day 1 and day 22 of each 42-day cycle. Pembrolizumab is administered IV on day 8 of each 42-day cycle at 400 mg. Primary and secondary outcomes are assessed as described above.

### Example 5: Treating Non-Squamous NSCLC with a Combination of SEA-CD40, Pembrolizumab, Carboplatin, and Pemetrexed

Treatment of non-squamous NSCLC is being assessed with the combination of SEA-CD40, pembrolizumab, carboplatin, and pemetrexed in a phase 2 clinical trial. Patients with non-squamous NSCLC are treated with a combination of SEA-CD40, pembrolizumab, carboplatin, and pemetrexed. SEA-CD40 is administered at a dose of 10 μg/kg on day 3 of each 21-day cycle. Pembrolizumab is administered on day 1 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC are treated with a combination of SEA-CD40, pembrolizumab, carboplatin, and pemetrexed. SEA-CD40 is administered at a dose of 10 g/kg on day 1 of each 21-day cycle. Pembrolizumab is administered on day 8 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC are treated with a combination of SEA-CD40, pembrolizumab, carboplatin, and pemetrexed. SEA-CD40 is administered at a dose of 10 μg/kg on day 3 of each 21-day cycle. Pembrolizumab is administered on day 8 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC are treated with a combination of SEA-CD40, pembrolizumab, carboplatin, and pemetrexed. SEA-CD40 is administered at a dose of 10 g/kg on day 3 of each 21-day cycle. Pembrolizumab is not administered in the first 21-day cycle, and is administered on day 1 of each 21-day cycle starting from the second 21-day cycle, at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC are treated with a combination of SEA-CD40, pembrolizumab, carboplatin, and pemetrexed. SEA-CD40 is administered at a dose of 10 g/kg on day 3 of each 21-day cycle. Pembrolizumab is not administered in the first 21-day cycle, and is administered on day 8 of each 21-day cycle starting from the second 21-day cycle, at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Primary outcome measure is confirmed Objective Response Rate (ORR). the proportion of subjects who achieve a confirmed complete response (CR) or partial response (PR) according to RECIST v1.1 per investigator assessment (Eisenhauer 2009). The timeframe is the duration of treatment, approximately two years.

Secondary outcome measure is type, incidence, severity, seriousness, and relatedness of adverse events (AEs). The incidence of adverse events (AEs) include any untoward medical occurrence in a subject or clinical investigational subject administered a medicinal product and which does not necessarily have a causal relationship with this treatment. The time frame is from the start of treatment to 30-37 days after last dose, approximately 2 years.

Secondary outcome measure also includes type, incidence, and severity of laboratory abnormalities. The incidence of laboratory abnormalities are to be summarized using descriptive statistics. The time frame is from the start of treatment to 30-37 days after last dose, approximately 2 years.

Secondary outcome measure also includes frequency of treatment interruptions, dose reductions, and treatment discontinuations due to AEs. The incidence of dose alterations is to be summarized using descriptive statistics. The time frame is the duration of treatment, approximately two years.

Secondary outcome measure also includes disease control rate (DCR; confirmed CR, PR, and stable disease [SD]) per investigator assessment. Disease control rate (DCR) per investigator assessment is the proportion of subjects who achieve a confirmed CR or PR according to RECIST v1.1 as assessed by the investigator or meet the stable disease (SD) criteria at least once after start of study treatment at a minimum interval of 5 weeks. The time frame is from the start of treatment until completion of response assessment, approximately 4 years.

Secondary outcome measure also includes duration of response (DOR) per investigator assessment: the time from the first documentation of objective response (CR or PR that is subsequently confirmed) to the first documentation of progressive disease (PD) or death due to any cause. The time frame is from the start of treatment until completion of response assessment, approximately 4 years.

Secondary outcome measure also includes progression-free survival (PFS) per investigator assessment: the time from the start of study treatment to the first documentation of PD by RECIST v1.1 or death due to any cause. The time frame is from the start of treatment until completion of response assessment, approximately 4 years.

Secondary outcome measure also includes overall survival (OS): the time from the start of study treatment to date of death due to any cause. The time frame is duration of study, approximately 4 years.

Eligibility criteria for non-squamous NSCLC may include: participants must have stage IV disease per AJCC 8th edition; no known driver mutations/alterations mutation for which targeted therapy is available; must have non-squamous histology; no prior therapy for metastatic disease; and no prior treatment with anti-PD-(L)1 or PD-L2 agent or an antibody targeting other immuno-regulatory receptors or mechanisms; able to provide archival tumor tissue from locations not radiated prior to biopsy; Eastern Cooperative Oncology Group (ECOG) Performance Status score of 0 or 1; and Measurable disease per RECIST v1.1 at baseline.

Exclusion criteria may include: history of another malignancy within 3 years of first dose of study drug; active central nervous system (CNS) metastases and/or carcinomatous meningitis; previous exposure to CD40-targeted therapy; currently on chronic systemic steroids in excess of physiologic replacement; has had an allogeneic tissue/solid organ transplant; history of autoimmune disease that has required systemic treatment in the past 2 years.

Eligible patients with non-squamous NSCLC are treated with a combination of SEA-CD40, pembrolizumab, carboplatin, and pemtrexed. SEA-CD40 is administered as an IV infusion at a dose of 10 meg/kg on Day 3 of each 21-day cycle. Pembrolizumab is administered IV on day 1 of each 21-day cycle at 200 mg. Pemetrexed is administered on day 1 of each 21-day cycle at a dose of 500 mg per square meter. Carboplatin is administered on Day 1 of Cycles 1-4 at a dose of area under the concentration-time curve (AUC) 5 mg per milliliter per minute.

### Example 6: Treating PD-L1 Low Expression Non-Squamous NSCLC with a Combination of SEA-CD40, Pembrolizumab, Carboplatin, and Pemetrexed

Although upfront chemoimmunotherapy has improved outcomes in metastatic NSCLC, there is a significant unmet need for improved response rates in patients with TPS<50%, especially in light of the fact that up to half of all NSCLC patients will quickly progress despite treatment and not have the opportunity to receive second-line therapy (Davies 2017). As such, improvement in front-line therapies in patients less responsive to chemoimmunotherapy represents an opportunity to improve survival in these patients by virtue of the ability for them to utilize second-line therapies upon failure.

Therefore, patients with non-squamous NSCLC are assessed for PD-L1 expression level. Some patients with a PD-L1 expression level of TPS<1% are treated with a combination of SEA-CD40, pembrolizumab, carboplatin, and pemetrexed. SEA-CD40 is administered at a dose of 10 μg/kg on day 3 of each 21-day cycle. Pembrolizumab is administered on day 1 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS<1% are treated with a combination of SEA-CD40, pembrolizumab, carboplatin, and pemetrexed. SEA-CD40 is administered at a dose of 10 μg/kg on day 1 of each 21-day cycle. Pembrolizumab is administered on day 8 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS<1% are treated with a combination of SEA-CD40, pembrolizumab, carboplatin, and pemetrexed. SEA-CD40 is administered at a dose of 10 μg/kg on day 3 of each 21-day cycle. Pembrolizumab is administered on day 8 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS<1% are treated with a combination of SEA-CD40, pembrolizumab, carboplatin, and pemetrexed. SEA-CD40 is administered at a dose of 10 μg/kg on day 3 of each 21-day cycle. Pembrolizumab is not administered in the first 21-day cycle, and is administered on day 1 of each 21-day cycle starting from the second 21-day cycle, at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS<1% are treated with a combination of SEA-CD40, pembrolizumab, carboplatin, and pemetrexed. SEA-CD40 is administered at a dose of 10 μg/kg on day 3 of each 21-day cycle. Pembrolizumab is not administered in the first 21-day cycle, and is administered on day 8 of each 21-day cycle starting from the second 21-day cycle, at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS 1-49% are treated with a combination of SEA-CD40, pembrolizumab, carboplatin, and pemetrexed. SEA-CD40 is administered at a dose of 10 μg/kg on day 3 of each 21-day cycle. Pembrolizumab is administered on day 1 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS 1-49% are treated with a combination of SEA-CD40, pembrolizumab, carboplatin, and pemetrexed. SEA-CD40 is administered at a dose of 10 μg/kg on day 1 of each 21-day cycle. Pembrolizumab is administered on day 8 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS 1-49% are treated with a combination of SEA-CD40, pembrolizumab, carboplatin, and pemetrexed. SEA-CD40 is administered at a dose of 10 μg/kg on day 3 of each 21-day cycle. Pembrolizumab is administered on day 8 of each 21-day cycle at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS 1-49% are treated with a combination of SEA-CD40, pembrolizumab, carboplatin, and pemetrexed. SEA-CD40 is administered at a dose of 10 μg/kg on day 3 of each 21-day cycle. Pembrolizumab is not administered in the first 21-day cycle, and is administered on day 1 of each 21-day cycle starting from the second 21-day cycle, at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Some patients with non-squamous NSCLC and with a PD-L1 expression level of TPS 1-49% are treated with a combination of SEA-CD40, pembrolizumab, carboplatin, and pemetrexed. SEA-CD40 is administered at a dose of 10 μg/kg on day 3 of each 21-day cycle. Pembrolizumab is not administered in the first 21-day cycle, and is administered on day 8 of each 21-day cycle starting from the second 21-day cycle, at 200 mg. Carboplatin is administered on day 1 of the first four 21-day cycles (cycles 1-4) at a dose of area under the concentration-time curve (AUC) 5 mg per milliliter per minute. Pemetrexed is administered on day 1 of each 21-day cycle at a dose of 500 mg per square meter.

Primary outcome measure is confirmed Objective Response Rate (ORR): the proportion of subjects who achieve a confirmed complete response (CR) or partial response (PR) according to RECIST v1.1 per investigator assessment (Eisenhauer 2009). The timeframe is the duration of treatment, approximately two years.

Secondary outcome measure is type, incidence, severity, seriousness, and relatedness of adverse events (AEs). The incidence of adverse events (AEs) include any untoward medical occurrence in a subject or clinical investigational subject administered a medicinal product and which does not necessarily have a causal relationship with this treatment. The time frame is from the start of treatment to 30-37 days after last dose, approximately 2 years.

Secondary outcome measure also includes type, incidence, and severity of laboratory abnormalities. The incidence of laboratory abnormalities are to be summarized using descriptive statistics. The time frame is from the start of treatment to 30-37 days after last dose, approximately 2 years.

Secondary outcome measure also includes frequency of treatment interruptions, dose reductions, and treatment discontinuations due to AEs. The incidence of dose alterations is to be summarized using descriptive statistics. The time frame is the duration of treatment, approximately two years.

Secondary outcome measure also includes disease control rate (DCR; confirmed CR, PR, and stable disease [SD]) per investigator assessment. Disease control rate (DCR) per investigator assessment is the proportion of subjects who achieve a confirmed CR or PR according to RECIST v1.1 as assessed by the investigator or meet the stable disease (SD) criteria at least once after start of study treatment at a minimum interval of 5 weeks. The time frame is from the start of treatment until completion of response assessment, approximately 4 years.

Secondary outcome measure also includes duration of response (DOR) per investigator assessment: the time from the first documentation of objective response (CR or PR that is subsequently confirmed) to the first documentation of progressive disease (PD) or death due to any cause. The time frame is from the start of treatment until completion of response assessment, approximately 4 years.

Secondary outcome measure also includes progression-free survival (PFS) per investigator assessment: the time from the start of study treatment to the first documentation of PD by RECIST v1.1 or death due to any cause. The time frame is from the start of treatment until completion of response assessment, approximately 4 years.

Secondary outcome measure also includes overall survival (OS): the time from the start of study treatment to date of death due to any cause. The time frame is duration of study, approximately 4 years.

Eligibility criteria for non-squamous NSCLC may include: participants must have stage IV disease per AJCC 8th edition; no known driver mutations/alterations mutation for which targeted therapy is available; must have non-squamous histology; no prior therapy for metastatic disease; and no prior treatment with anti-PD-(L)1 or PD-L2 agent or an antibody targeting other immuno-regulatory receptors or mechanisms; able to provide archival tumor tissue from locations not radiated prior to biopsy; Eastern Cooperative Oncology Group (ECOG) Performance Status score of 0 or 1; and Measurable disease per RECIST v1.1 at baseline.

Exclusion criteria may include: history of another malignancy within 3 years of first dose of study drug; active central nervous system (CNS) metastases and/or carcinomatous meningitis; previous exposure to CD40-targeted therapy; currently on chronic systemic steroids in excess of physiologic replacement; has had an allogeneic tissue/solid organ transplant; history of autoimmune disease that has required systemic treatment in the past 2 years.

Eligible patients with non-squamous NSCLC and with a PD-L1 expression level of either <1% or between 1-49% are treated with a combination of SEA-CD40, pembrolizumab, carboplatin, and pemtrexed. SEA-CD40 is administered as an IV infusion at a dose of 10 mcg/kg on Day 3 of each 21-day cycle. Pembrolizumab is administered IV on day 1 of each 21-day cycle at 200 mg. Pemetrexed is administered on day 1 of each 21-day cycle at a dose of 500 mg per square meter. Carboplatin is administered on Day 1 of Cycles 1-4 at a dose of area under the concentration-time curve (AUC) 5 mg per milliliter per minute.

### Example 7: Treating Squamous NSCLC with a Combination of SEA-CD40, Pembrolizumab, Carboplatin, and Paclitaxel or Nab-Paclitaxel

Treatment of squamous NSCLC is assessed with the combination of SEA-CD40, pembrolizumab, carboplatin, and paclitaxel or nab-paclitaxel. Some patients with squamous NSCLC are treated with a combination of SEA-CD40, pembrolizumab, carboplatin, and paclitaxel. Some patients with squamous NSCLC are treated with a combination of SEA-CD40, pembrolizumab, carboplatin, and nab-paclitaxel. SEA-CD40 is administered at a dose of 10 g/kg on day 3 of each 21-day cycle. Pembrolizumab is administered on day 1 of each 21-day cycle at 200 mg. Carboplatin is administered on day 1 of the first four 21-day cycles (cycles 1-4) at a dose of area under the concentration-time curve (AUC) 6 mg per milliliter per minute. Paclitaxel is administered at 200 mg per square meter of body-surface area on day 1 of each 21-day cycle. Nab-paclitaxel is administered at 100 mg per square meter on days 1, 8, and 15 of each 21-day cycle.

Some patients with squamous NSCLC are treated with a combination of SEA-CD40, pembrolizumab, carboplatin, and paclitaxel; and some patients with squamous NSCLC are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is administered on day 8 of each 21-day cycle at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC are treated with a combination of SEA-CD40, pembrolizumab, carboplatin, and paclitaxel; and some patients with squamous NSCLC are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is not administered in the first 21-day cycle, and is administered on day 1 of each 21-day cycle starting from the second 21-day cycle, at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC are treated with a combination of SEA-CD40, pembrolizumab, carboplatin, and paclitaxel; and some patients with squamous NSCLC are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is not administered in the first 21-day cycle, and is administered on day 8 of each 21-day cycle starting from the second 21-day cycle, at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Outcomes are evaluated by using Objective Response Rate (ORR): the proportion of subjects who achieve a confirmed complete response (CR) or partial response (PR) according to RECIST v1.1 per investigator assessment (Eisenhauer 2009).

### Example 8: Treating PD-L1 Low Expression Squamous NSCLC with a Combination of SEA-CD40, Pembrolizumab, Carboplatin, and Paclitaxel or Nab-Paclitaxel

Although upfront chemoimmunotherapy has improved outcomes in metastatic NSCLC, there is a significant unmet need for improved response rates in patients with TPS<50%, especially in light of the fact that up to half of all NSCLC patients will quickly progress despite treatment and not have the opportunity to receive second-line therapy (Davies 2017). As such, improvement in front-line therapies in patients less responsive to chemoimmunotherapy represents an opportunity to improve survival in these patients by virtue of the ability for them to utilize second-line therapies upon failure.

Therefore, patients with squamous NSCLC is assessed for PD-L1 expression level. Some squamous NSCLC patients with a PD-L1 expression level of TPS<1% are treated with a combination of SEA-CD40, pembrolizumab, carboplatin, and paclitaxel. Some squamous NSCLC patients with a PD-L1 expression level of TPS<1% are treated with a combination of SEA-CD40, pembrolizumab, carboplatin, and nab-paclitaxel. SEA-CD40 is administered at a dose of 10 μg/kg on day 3 of each 21-day cycle. Pembrolizumab is administered on day 1 of each 21-day cycle at 200 mg. Carboplatin is administered on day 1 of the first four 21-day cycles (cycles 1-4) at a dose of area under the concentration-time curve (AUC) 6 mg per milliliter per minute. Paclitaxel is administered at 200 mg per square meter of body-surface area on day 1 of each 21-day cycle. Nab-paclitaxel is administered at 100 mg per square meter on days 1, 8, and 15 of each 21-day cycle.

Some patients with squamous NSCLC and with a PD-L1 expression level of TPS<1% are treated with a combination of SEA-CD40, pembrolizumab, carboplatin, and paclitaxel; and some patients with squamous NSCLC and with a PD-L1 expression level of TPS<1% are treated with a combination of SEA-CD40, pembrolizumab, carboplatin, and nab-paclitaxel; at the following doses and schedules: SEA-CD40 is administered at a dose of 10 μg/kg on day 1 of each 21-day cycle; pembrolizumab is administered on day 8 of each 21-day cycle at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS<1% are treated with a combination of SEA-CD40, pembrolizumab, carboplatin, and paclitaxel; and some patients with squamous NSCLC and with a PD-L1 expression level of TPS<1% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is administered on day 8 of each 21-day cycle at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS<1% are treated with a combination of SEA-CD40, pembrolizumab, carboplatin, and paclitaxel; and some patients with squamous NSCLC and with a PD-L1 expression level of TPS<1% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is not administered in the first 21-day cycle and is administered on day 1 of each 21-day cycle starting from the second 21-day cycle, at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS<1% are treated with a combination of SEA-CD40, pembrolizumab, carboplatin, and paclitaxel; and some patients with squamous NSCLC and with a PD-L1 expression level of TPS<1% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is not administered in the first 21-day cycle and is administered on day 8 of each 21-day cycle starting from the second 21-day cycle, at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS 1-49% are treated with a combination of SEA-CD40, pembrolizumab, carboplatin, and paclitaxel; and some patients with squamous NSCLC and with a PD-L1 expression level of TPS 1-49% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is administered on day 1 of each 21-day cycle at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS 1-49% are treated with a combination of SEA-CD40, pembrolizumab, carboplatin, and paclitaxel; and some patients with squamous NSCLC and with a PD-L1 expression level of TPS 1-49% are treated with a combination of SEA-CD40, pembrolizumab, carboplatin, and nab-paclitaxel; at the following doses and schedules: SEA-CD40 is administered at a dose of 10 μg/kg on day 1 of each 21-day cycle; pembrolizumab is administered on day 8 of each 21-day cycle at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS 1-49% are treated with a combination of SEA-CD40, pembrolizumab, carboplatin, and paclitaxel; and some patients with squamous NSCLC and with a PD-L1 expression level of TPS 1-49% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is administered on day 8 of each 21-day cycle at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS 1-49% are treated with a combination of SEA-CD40, pembrolizumab, carboplatin, and paclitaxel; and some patients with squamous NSCLC and with a PD-L1 expression level of TPS 1-49% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is not administered in the first 21-day cycle and is administered on day 1 of each 21-day cycle starting from the second 21-day cycle, at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Some patients with squamous NSCLC and with a PD-L1 expression level of TPS 1-49% are treated with a combination of SEA-CD40, pembrolizumab, carboplatin, and paclitaxel; and some patients with squamous NSCLC and with a PD-L1 expression level of TPS 1-49% are treated with a combination of SEA-CD40, pembrolizumab, carboplatin, and nab-paclitaxel; at the following doses and schedules: SEA-CD40 is administered at a dose of 10 μg/kg on day 3 of each 21-day cycle; pembrolizumab is not administered in the first 21-day cycle and is administered on day 8 of each 21-day cycle starting from the second 21-day cycle, at 200 mg; carboplatin is administered on day 1 of the first four 21-day cycles (cycles 1-4) at a dose of area under the concentration-time curve (AUC) 6 mg per milliliter per minute; paclitaxel is administered at 200 mg per square meter of body-surface area on day 1 of each 21-day cycle; and nab-paclitaxel is administered at 100 mg per square meter on days 1, 8, and 15 of each 21-day cycle.

Outcomes are evaluated by using Objective Response Rate (ORR): the proportion of subjects who achieve a confirmed complete response (CR) or partial response (PR) according to RECIST v1.1 per investigator assessment (Eisenhauer 2009).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a melanoma comprising administering to a patient having melanoma SEA-CD40 and pembrolizumab wherein SEA-CD40 and pembrolizumab are both administered in 42-day cycles.

2. The method of claim 1 wherein SEA-CD40 is administered twice in each 42-day cycle and pembrolizumab is administered once in each 42-day cycle.

3. The method of claim 2 wherein the first administration of SEA-CD40, the second administration of SEA-CD40 and the administration of pembrolizumab are on the following days of each 42-day cycle, respectively:
day 1, day 21, and day 5;
day 1, day 22, and day 5;
day 1, day 23, and day 5;
day 2, day 21, and day 5;
day 2, day 22, and day 5;
day 2, day 23, and day 5;
day 3, day 21, and day 5;
day 3, day 22, and day 5;
day 3, day 23, and day 5;
day 1, day 21, and day 6;
day 1, day 22, and day 6;
day 1, day 23, and day 6;
day 2, day 21, and day 6;
day 2, day 22, and day 6;
day 2, day 23, and day 6;
day 3, day 21, and day 6;
day 3, day 22, and day 6;
day 3, day 23, and day 6;
day 1, day 21, and day 7;
day 1, day 22, and day 7;
day 1, day 23, and day 7;
day 2, day 21, and day 7;
day 2, day 22, and day 7;
day 2, day 23, and day 7;
day 3, day 21, and day 7;
day 3, day 22, and day 7;
day 3, day 23, and day 7;
day 1, day 21, and day 8;
day 1, day 22, and day 8;
day 1, day 23, and day 8;
day 2, day 21, and day 8;
day 2, day 22, and day 8;
day 2, day 23, and day 8;
day 3, day 21, and day 8;
day 3, day 22, and day 8;
day 3, day 23, and day 8;
day 1, day 21, and day 9;
day 1, day 22, and day 9;
day 1, day 23, and day 9;
day 2, day 21, and day 9;
day 2, day 22, and day 9;
day 2, day 23, and day 9;
day 3, day 21, and day 9;
day 3, day 22, and day 9;
day 3, day 23, and day 9;
day 1, day 21, and day 10;
day 1, day 22, and day 10;
day 1, day 23, and day 10;
day 2, day 21, and day 10;
day 2, day 22, and day 10;
day 2, day 23, and day 10;
day 3, day 21, and day 10;
day 3, day 22, and day 10; or
day 3, day 23, and day 10.

4. The method of any one of claims 1-3, wherein SEA-CD40 is administered on day 1 and day 21 of each 42-day cycle, and pembrolizumab is administered on day 8 of each 42-day cycle; or wherein SEA-CD40 is administered on day 1 and day 22 of each 42-day cycle, and pembrolizumab is administered on day 8 of each 42-day cycle.

5. The method of claim 1 or 2 wherein pembrolizumab is not administered in the first 42-day cycle and is administered each cycle starting from the second cycle.

6. The method of claim 5 wherein SEA-CD40 is administered twice in each 42-day cycle and pembrolizumab is not administered in the first cycle and is administered once in each 42-day cycle starting from the second cycle.

7. The method of claim 5 or 6 wherein the first administration of SEA-CD40, the second administration of SEA-CD40 in each 42-day cycle, and the administration of pembrolizumab in each 42-day cycle starting from the second cycle are on the following days of the 42-day cycles, respectively:
day 1, day 21, and day 5;
day 1, day 22, and day 5;
day 1, day 23, and day 5;
day 2, day 21, and day 5;
day 2, day 22, and day 5;
day 2, day 23, and day 5;
day 3, day 21, and day 5;
day 3, day 22, and day 5;
day 3, day 23, and day 5;
day 1, day 21, and day 6;
day 1, day 22, and day 6;
day 1, day 23, and day 6;
day 2, day 21, and day 6;
day 2, day 22, and day 6;
day 2, day 23, and day 6;
day 3, day 21, and day 6;
day 3, day 22, and day 6;
day 3, day 23, and day 6;
day 1, day 21, and day 7;
day 1, day 22, and day 7;
day 1, day 23, and day 7;
day 2, day 21, and day 7;
day 2, day 22, and day 7;
day 2, day 23, and day 7;
day 3, day 21, and day 7;
day 3, day 22, and day 7;
day 3, day 23, and day 7;
day 1, day 21, and day 8;
day 1, day 22, and day 8;
day 1, day 23, and day 8;
day 2, day 21, and day 8;
day 2, day 22, and day 8;
day 2, day 23, and day 8;
day 3, day 21, and day 8;
day 3, day 22, and day 8;
day 3, day 23, and day 8;
day 1, day 21, and day 9;
day 1, day 22, and day 9;
day 1, day 23, and day 9;
day 2, day 21, and day 9;
day 2, day 22, and day 9;
day 2, day 23, and day 9;
day 3, day 21, and day 9;
day 3, day 22, and day 9;
day 3, day 23, and day 9;
day 1, day 21, and day 10;
day 1, day 22, and day 10;
day 1, day 23, and day 10;
day 2, day 21, and day 10;
day 2, day 22, and day 10;
day 2, day 23, and day 10;
day 3, day 21, and day 10;
day 3, day 22, and day 10; or
day 3, day 23, and day 10.

8. The method of any one of claims 1-7 wherein pembrolizumab is administered intravenously.

9. The method of any one of claims 1-8 wherein pembrolizumab is administered at a dose of 400 mg.

10. The method of any one of claims 1-9 wherein the melanoma is relapsed and/or refractory melanoma.

11. The method of any one of claims 1-9 wherein the melanoma is uveal melanoma.

12. The method of any one of claims 1-9 wherein the melanoma is PD-(L)1-naïve melanoma.

13. The method of any one of claims 1-12 wherein the level of PD-L1 in a sample of the melanoma is less than 50% as measured by Total Proportion Score (TPS).

14. The method of any one of claims claim 1-13 wherein the level of PD-L1 in a sample of the melanoma is less than 1% as measured by TPS.

15. The method of any one of claims claim 1-13 wherein the level of PD-L1 in a sample of the melanoma is 1-49% as measured by TPS.

16. The method of any one of the claims 1-15 further comprising administering one of more chemotherapy agents to the patient.

17. The method of claim 16 wherein the one or more chemotherapy agents comprises gemcitabine, dacarbazine, temozolomide, paclitaxel, albumin-bound paclitaxel, carboplatin, and/or pemetrexed.

18. The method of claim 17 wherein the albumin-bound paclitaxel is nab-paclitaxel.

19. A method of treating a melanoma comprising administering to a patient having melanoma SEA-CD40 and pembrolizumab wherein SEA-CD40 and pembrolizumab are both administered in 21-day cycles.

20. The method of claim 19 wherein both SEA-CD40 and pembrolizumab are administered once in each 21-day cycle.

21. The method of claim 19 or 20 wherein SEA-CD40 and pembrolizumab are administered on the following days of each 21-day cycle, respective:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 2 and day 11,
day 3 and day 4,
day 3 and day 5,
day 3 and day 6,
day 3 and day 7,
day 3 and day 8,
day 3 and day 9,
day 3 and day 10,
day 3 and day 11,
day 3 and day 12,
day 4 and day 5,
day 4 and day 6,
day 4 and day 7,
day 4 and day 8,
day 4 and day 9,
day 4 and day 10,
day 4 and day 11,
day 4 and day 12,
day 4 and day 13,
day 5 and day 6,
day 5 and day 7,
day 5 and day 8,
day 5 and day 9,
day 5 and day 10,
day 5 and day 11,
day 5 and day 12,
day 5 and day 13, or
day 5 and day 14.

22. The method of any one of claims 19-21, wherein SEA-CD40 is administered on day 1 of each 21-day cycle, and pembrolizumab is administered on day 8 of each 21-day cycle.

23. The method of claim 19 or 20, wherein pembrolizumab is not administered in the first 21-day cycle and is administered each cycle starting from the second cycle.

24. The method of claim 23 wherein SEA-CD40 is administered once in each 42-day cycle and pembrolizumab is not administered in the first cycle and is administered once in each 21-day cycle starting from the second cycle.

25. The method of claim 23 or 24 wherein the administration of SEA-CD40 in each 21-day cycle, and the administration of pembrolizumab are on the following days of the 21-day cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 2 and day 11,
day 3 and day 4,
day 3 and day 5,
day 3 and day 6,
day 3 and day 7,
day 3 and day 8,
day 3 and day 9,
day 3 and day 10,
day 3 and day 11,
day 3 and day 12,
day 4 and day 5,
day 4 and day 6,
day 4 and day 7,
day 4 and day 8,
day 4 and day 9,
day 4 and day 10,
day 4 and day 11,
day 4 and day 12,
day 4 and day 13,
day 5 and day 6,
day 5 and day 7,
day 5 and day 8,
day 5 and day 9,
day 5 and day 10,
day 5 and day 11,
day 5 and day 12,
day 5 and day 13, or
day 5 and day 14.

26. The method of any one of claims 19-25 wherein pembrolizumab is administered intravenously.

27. The method of any one of claims 19-26 wherein pembrolizumab is administered at a dose of 400 mg.

28. The method of any one of claims 19-27 wherein the melanoma is relapsed and/or refractory melanoma.

29. The method of any one of claims 19-27 wherein the melanoma is uveal melanoma.

30. The method of any one of claims 19-27 wherein the melanoma is PD-(L)1-naïve melanoma.

31. The method of any one of claims 19-30 wherein the level of PD-L1 in a sample of the melanoma is less than 50% as measured by Total Proportion Score (TPS).

32. The method of any one of claims claim 19-31 wherein the level of PD-L1 in a sample of the melanoma is less than 1% as measured by TPS.

33. The method of any one of claims claim 19-31 wherein the level of PD-L1 in a sample of the melanoma is 1-49% as measured by TPS.

34. The method of any one of the claims 19-33 further comprising administering one of more chemotherapy agents to the patient.

35. The method of claim 34 wherein the one or more chemotherapy agents comprises gemcitabine, dacarbazine, temozolomide, paclitaxel, albumin-bound paclitaxel, carboplatin, and/or pemetrexed.

36. The method of claim 35 wherein the albumin-bound paclitaxel is nab-paclitaxel.

37. A method of treating non-squamous NSCLC comprising administering to a patient having non-squamous NSCLC SEA-CD40, pembrolizumab, carboplatin, and pemetrexed.

38. The method of claim 37 wherein SEA-CD40, pembrolizumab, carboplatin and pemetrexed are all administered in 21-day cycles.

39. The method of claim 38 wherein carboplatin is administered on day 1 of the first four cycles (cycles 1-4) and not administered in the following cycles.

40. The method of claim 38 or 39 wherein pemetrexed is administered on day 1 of each 21-day cycle.

41. The method of any one of claims 38-40 wherein SEA-CD40 and pembrolizumab are administered on the following days of each 21-day cycle, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

42. The method of any one of claims 38-41, wherein SEA-CD40 and pembrolizumab are administered on the following days of each 21-day cycle, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

43. The method of any one of claims 38-41, wherein SEA-CD40 is administered on day 3 of each 21-day cycle, and pembrolizumab is administered on day 1 of each 21-day cycle.

44. The method of any one of claims 38-40, wherein pembrolizumab is not administered in the first 21-day cycle and is administered each cycle starting from the second 21-day cycle.

45. The method of claim 44, wherein the administration of SEA-CD40 in each 21-day cycle, and the administration of pembrolizumab in each 21-day cycle except the first cycle, are on the following days of the 21-day cycles, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

46. The method of claim 44 or 45, wherein the administration of SEA-CD40 in each 21-day cycle, and the administration of pembrolizumab in each 21-day cycle except the first cycle, are on the following days of the 21-day cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

47. The method of any one of claims 38-46 wherein pembrolizumab is administered intravenously.

48. The method of any one of claims 38-47 wherein pembrolizumab is administered at a dose of 200 mg.

49. The method of any one of claims 38-48, wherein pemetrexed is administered at a dose of 500 mg per square meter.

50. The method of any one of claims 38-49, wherein carboplatin is administered at a dose of area under the concentration-time curve (AUC) 2-8 mg per milliliter per minute.

51. The method of any one of claims 38-50, wherein carboplatin is administered at a dose of area under the concentration-time curve (AUC) 2 mg, 3 mg, 4 mg, 5 mg, 6 mg, 7 mg, or 8 mg per milliliter per minute.

52. The method of any one of claims 38-51, wherein carboplatin is administered at 5 mg per milliliter per minute.

53. The method of any one of claims 38-52 wherein the level of PD-L1 in a sample of the non-squamous NSCLC is less than 50% as measured by Total Proportion Score (TPS).

54. The method of any one of claims claim 38-53 wherein the level of PD-L1 in a sample of the non-squamous NSCLC is less than 1% as measured by TPS.

55. The method of any one of claims claim 38-53 wherein the level of PD-L1 in a sample of the non-squamous NSCLC is 1-49% as measured by TPS.

56. The method of any one of the claims 38-55 further comprising administering gemcitabine, dacarbazine, temozolomide, paclitaxel, and/or albumin-bound paclitaxel.

57. The method of claim 56 wherein the albumin-bound paclitaxel is nab-paclitaxel.

58. A method of treating non-squamous NSCLC comprising administering to a patient having non-squamous NSCLC SEA-CD40, pembrolizumab, carboplatin, and pemetrexed, wherein SEA-CD40, carboplatin and pemetrexed are administered in 21-day cycles, and pembrolizumab is administered in 42-day cycles.

59. The method of claim 58 wherein carboplatin is administered on day 1 of the first four cycles (cycles 1-4) and not administered in the following cycles.

60. The method of claim 58 or 59 wherein pemetrexed is administered on day 1 of each 21-day cycle.

61. The method of any one of claims 58-60 wherein SEA-CD40 and pembrolizumab are administered on the following days in each of their respective cycles, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

62. The method of any one of claims 58-61, wherein SEA-CD40 and pembrolizumab are administered on the following days in each of their respective cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

63. The method of any one of claims 58-61, wherein SEA-CD40 is administered on day 3 of each 21-day cycle, and pembrolizumab is administered on day 1 of each 42-day cycle.

64. The method of any one of claims 58-60, wherein pembrolizumab is not administered in the first 42-day cycle and is administered each cycle starting from the second 42-day cycle.

65. The method of claim 64, wherein the administration of SEA-CD40 in each 21-day cycle, and the administration of pembrolizumab in each 42-day cycle except the first cycle, are on the following days of their respective cycles, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

66. The method of claim 64 or 65, wherein the administration of SEA-CD40 in each 21-day cycle, and the administration of pembrolizumab in each 42-day cycle except the first cycle, are on the following days of their respective cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

67. The method of any one of claims 58-66 wherein pembrolizumab is administered intravenously.

68. The method of any one of claims 58-67 wherein pembrolizumab is administered at a dose of 400 mg.

69. The method of any one of claims 58-68, wherein pemetrexed is administered at a dose of 500 mg per square meter.

70. The method of any one of claims 58-69, wherein carboplatin is administered at a dose of area under the concentration-time curve (AUC) 2-8 mg per milliliter per minute.

71. The method of any one of claims 58-70, wherein carboplatin is administered at a dose of area under the concentration-time curve (AUC) 2 mg, 3 mg, 4 mg, 5 mg, 6 mg, 7 mg, or 8 mg per milliliter per minute.

72. The method of any one of claims 58-71, wherein carboplatin is administered at 5 mg per milliliter per minute.

73. The method of any one of claims 58-72 wherein the level of PD-L1 in a sample of the non-squamous NSCLC is less than 50% as measured by Total Proportion Score (TPS).

74. The method of any one of claims claim 58-73 wherein the level of PD-L1 in a sample of the non-squamous NSCLC is less than 1% as measured by TPS.

75. The method of any one of claims claim 58-73 wherein the level of PD-L1 in a sample of the non-squamous NSCLC is 1-49% as measured by TPS.

76. The method of any one of the claims 58-75 further comprising administering gemcitabine, dacarbazine, temozolomide, paclitaxel, and/or albumin-bound paclitaxel.

77. The method of claim 76 wherein the albumin-bound paclitaxel is nab-paclitaxel.

78. A method of treating squamous NSCLC comprising administering to a patient having squamous NSCLC: 1) SEA-CD40, 2) pembrolizumab, 3) carboplatin, and 4) paclitaxel or nab-paclitaxel.

79. The method of claim 78 wherein SEA-CD40, pembrolizumab, carboplatin and paclitaxel or nab-paclitaxel are all administered in 21-day cycles.

80. The method of claim 79 wherein carboplatin is administered on day 1 of the first four cycles (cycles 1-4) and not administered in the following cycles.

81. The method of claim 79 or 80 wherein paclitaxel, when administered, is administered on day 1 of each 21-day cycle at a dose of 200 mg per square meter of body-surface area; and nab-paclitaxel, when administered, is administered on days 1, 8, and 15 at a dose of 100 mg per square meter.

82. The method of any one of claims 79-81 wherein SEA-CD40 and pembrolizumab are administered on the following days of each 21-day cycle, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

83. The method of any one of claims 79-82, wherein SEA-CD40 and pembrolizumab are administered on the following days of each 21-day cycle, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

84. The method of any one of claims 79-82, wherein SEA-CD40 is administered on day 3 of each 21-day cycle, and pembrolizumab is administered on day 1 of each 21-day cycle.

85. The method of any one of claims 79-81, wherein pembrolizumab is not administered in the first 21-day cycle and is administered each cycle starting from the second 21-day cycle.

86. The method of claim 85, wherein the administration of SEA-CD40 in each 21-day cycle, and the administration of pembrolizumab in each 21-day cycle except the first cycle, are on the following days of the 21-day cycles, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

87. The method of claim 85 or 86, wherein the administration of SEA-CD40 in each 21-day cycle, and the administration of pembrolizumab in each 21-day cycle except the first cycle, are on the following days of the 21-day cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

88. The method of any one of claims 79-87 wherein pembrolizumab is administered intravenously.

89. The method of any one of claims 79-88 wherein pembrolizumab is administered at a dose of 200 mg.

90. The method of any one of claims 79-89, wherein carboplatin is administered at a dose of area under the concentration-time curve (AUC) 2-8 mg per milliliter per minute.

91. The method of any one of claims 79-90, wherein carboplatin is administered at a dose of area under the concentration-time curve (AUC) 2 mg, 3 mg, 4 mg, 5 mg, 6 mg, 7 mg, or 8 mg per milliliter per minute.

92. The method of any one of claims 79-91, wherein carboplatin is administered at 6 mg per milliliter per minute.

93. The method of any one of claims 79-92 wherein the level of PD-L1 in a sample of the squamous NSCLC is less than 50% as measured by Total Proportion Score (TPS).

94. The method of any one of claims claim 79-93 wherein the level of PD-L1 in a sample of the squamous NSCLC is less than 1% as measured by TPS.

95. The method of any one of claims claim 79-93 wherein the level of PD-L1 in a sample of the squamous NSCLC is 1-49% as measured by TPS.

96. The method of any one of the claims 79-95 further comprising administering gemcitabine, dacarbazine, temozolomide, or pemetrexed.

97. A method of treating squamous NSCLC comprising administering to a patient having squamous NSCLC: 1) SEA-CD40, 2) pembrolizumab, 3) carboplatin, and 4) paclitaxel or nab-paclitaxel, wherein SEA-CD40, carboplatin and paclitaxel or nab-paclitaxel are administered in 21-day cycles and pembrolizumab is administered in 42-day cycles.

98. The method of claim 97 wherein carboplatin is administered on day 1 of the first four cycles (cycles 1-4) and not administered in the following cycles.

99. The method of claim 97 or 98 wherein paclitaxel, when administered, is administered on day 1 of each 21-day cycle at a dose of 200 mg per square meter of body-surface area; and nab-paclitaxel, when administered, is administered on days 1, 8, and 15 at a dose of 100 mg per square meter.

100. The method of any one of claims 97-99 wherein SEA-CD40 and pembrolizumab are administered on the following days of their respective cycles, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

101. The method of any one of claims 97-100, wherein SEA-CD40 and pembrolizumab are administered on the following days of their respective cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

102. The method of any one of claims 97-100, wherein SEA-CD40 is administered on day 3 of each 21-day cycle, and pembrolizumab is administered on day 1 of each 242-day cycle.

103. The method of any one of claims 97-99, wherein pembrolizumab is not administered in the first 42-day cycle and is administered each cycle starting from the second 42-day cycle.

104. The method of claim 103, wherein the administration of SEA-CD40 in each 21-day cycle, and the administration of pembrolizumab in each 42-day cycle except the first cycle, are on the following days of their respective cycles, respectively:
day 1 and day 1,
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 1,
day 2 and day 2,
day 2 and day 3,
day 2 and day 4,
day 2 and day 5,
day 2 and day 6,
day 2 and day 7,
day 2 and day 8,
day 2 and day 9,
day 2 and day 10,
day 3 and day 1,
day 3 and day 2,
day 3 and day 3,
day 3 and day 4,
day 3 and day 5,
day 3 and day 6,
day 3 and day 7,
day 3 and day 8,
day 3 and day 9,
day 3 and day 10,
day 4 and day 1,
day 4 and day 2,
day 4 and day 3,
day 4 and day 4,
day 4 and day 5,
day 4 and day 6,
day 4 and day 7,
day 4 and day 8,
day 4 and day 9,
day 4 and day 10,
day 5 and day 1,
day 5 and day 2,
day 5 and day 3,
day 5 and day 4,
day 5 and day 5,
day 5 and day 6,
day 5 and day 7,
day 5 and day 8,
day 5 and day 9,
day 5 and day 10,
day 6 and day 1,
day 6 and day 2,
day 6 and day 3,
day 6 and day 4,
day 6 and day 5,
day 6 and day 6,
day 6 and day 7,
day 6 and day 8,
day 6 and day 9,
day 6 and day 10,
day 7 and day 1,
day 7 and day 2,
day 7 and day 3,
day 7 and day 4,
day 7 and day 5,
day 7 and day 6,
day 7 and day 7,
day 7 and day 8,
day 7 and day 9,
day 7 and day 10,
day 8 and day 1,
day 8 and day 2,
day 8 and day 3,
day 8 and day 4,
day 8 and day 5,
day 8 and day 6,
day 8 and day 7,
day 8 and day 8,
day 8 and day 9,
day 8 and day 10,
day 9 and day 1,
day 9 and day 2,
day 9 and day 3,
day 9 and day 4,
day 9 and day 5,
day 9 and day 6,
day 9 and day 7,
day 9 and day 8,
day 9 and day 9,
day 9 and day 10,
day 10 and day 1,
day 10 and day 2,
day 10 and day 3,
day 10 and day 4,
day 10 and day 5,
day 10 and day 6,
day 10 and day 7,
day 10 and day 8,
day 10 and day 9, or
day 10 and day 10.

105. The method of claim 103 or 104, wherein the administration of SEA-CD40 in each 21-day cycle, and the administration of pembrolizumab in each 42-day cycle except the first cycle, are on the following days of their respective cycles, respectively:
day 1 and day 2,
day 1 and day 3,
day 1 and day 4,
day 1 and day 5,
day 1 and day 6,
day 1 and day 7,
day 1 and day 8,
day 1 and day 9,
day 1 and day 10,
day 2 and day 3,
day 2 and day 4,
day 2 and day 5,
day 2 and day 6,
day 2 and day 7,
day 2 and day 8,
day 2 and day 9,
day 2 and day 10,
day 3 and day 4,
day 3 and day 5,
day 3 and day 6,
day 3 and day 7,
day 3 and day 8,
day 3 and day 9,
day 3 and day 10,
day 4 and day 5,
day 4 and day 6,
day 4 and day 7,
day 4 and day 8,
day 4 and day 9,
day 4 and day 10,
day 5 and day 6,
day 5 and day 7,
day 5 and day 8,
day 5 and day 9, or
day 5 and day 10.

106. The method of any one of claims 97-105 wherein pembrolizumab is administered intravenously.

107. The method of any one of claims 97-106 wherein pembrolizumab is administered at a dose of 400 mg.

108. The method of any one of claims 97-107, wherein carboplatin is administered at a dose of area under the concentration-time curve (AUC) 2-8 mg per milliliter per minute.

109. The method of any one of claims 97-108, wherein carboplatin is administered at a dose of area under the concentration-time curve (AUC) 2 mg, 3 mg, 4 mg, 5 mg, 6 mg, 7 mg, or 8 mg per milliliter per minute.

110. The method of any one of claims 97-109, wherein carboplatin is administered at 6 mg per milliliter per minute.

111. The method of any one of claims 97-110 wherein the level of PD-L1 in a sample of the squamous NSCLC is less than 50% as measured by Total Proportion Score (TPS).

112. The method of any one of claims 97-111 wherein the level of PD-L1 in a sample of the squamous NSCLC is less than 1% as measured by TPS.

113. The method of any one of claims 97-111 wherein the level of PD-L1 in a sample of the squamous NSCLC is 1-49% as measured by TPS.

114. The method of any one of the claims 97-113 further comprising administering gemcitabine, dacarbazine, temozolomide, or pemetrexed.

115. The method of any one of claims 1-114, wherein SEA-CD40 comprises a heavy chain variable region comprising amino acid 1-113 of SEQ ID NO: 1, and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue.

116. The method of claim 115 wherein less than 10% of N-glycoside-linked sugar chains in the composition comprise a fucose residue.

117. The method of claim 115 or 116 wherein less than 5% of N-glycoside-linked sugar chains in the composition comprise a fucose residue.

118. The method of any one of claims 115-117 wherein less than 3% of N-glycoside-linked sugar chains in the composition comprise a fucose residue.

119. The method of any one of claims 115-118 wherein less than 2% of N-glycoside-linked sugar chains in the composition comprise a fucose residue.

120. The method of any one of claims 115-119 wherein the anti-CD40 antibody comprises a heavy chain comprising the amino acid sequence of SEQ ID NO: 1 and a light chain comprising the amino acid sequence of SEQ ID NO: 2.

121. The method of any one of claims 1-120, wherein pembrolizumab comprises a light chain comprising CDRs of SEQ ID NOs: 3-5, and a heavy chain comprising CDRs of SEQ ID NOs 8-10.

122. The method of any one of claims 1-121 wherein pembrolizumab comprises a light chain variable region comprising the amino acid sequence of SEQ ID NO: 6, and a heavy chain variable region comprising the amino acid sequence of SEQ ID NO: 11.

123. The method of any one of claims 1-122 wherein pembrolizumab comprises a light chain comprising the amino acid sequence of SEQ ID NO: 7, and a heavy chain comprising the amino acid sequence of SEQ ID NO: 12.

124. The method of any one of claims 1-123 wherein SEA-CD40 is administered intravenously.

125. The method of any one of claims 1-124 wherein SEA-CD40 is administered at a dose of 10 μg/kg.

126. The method of any one of claims 1-124 wherein SEA-CD40 is administered at a dose of 30 μg/kg.


SEQUENCE LISTING (12 sequences)

SEQ ID NO: 1 - protein, 444 aa, synthetic construct
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, synthetic construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 15 aa, synthetic construct
RASKGVSTSGYSYLH

SEQ ID NO: 4 - protein, 7 aa, synthetic construct
LASYLES

SEQ ID NO: 5 - protein, 9 aa, synthetic construct
QHSRDLPLT

SEQ ID NO: 6 - protein, 111 aa, synthetic construct [sequence omitted]

SEQ ID NO: 7 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 5 aa, synthetic construct
NYYMY

SEQ ID NO: 9 - protein, 17 aa, synthetic construct
GINPSNGGTNFNEKFKN

SEQ ID NO: 10 - protein, 11 aa, synthetic construct
RDYRFDMGFDY

SEQ ID NO: 11 - protein, 120 aa, synthetic construct [sequence omitted]

SEQ ID NO: 12 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK